(19) United States
(12) Patent Application Publication
(10) Pub. No.: US 2021/0363192 A1
(43) Pub. Date: Nov. 25, 2021

(54) ENGINEERED AAV CAPSIDS WITH INCREASED TROPISM AND AAV VECTORS COMPRISING THE ENGINEERED CAPSIDS AND METHODS OF MAKING AND USING SAME

(71) Applicant: SPARK THERAPEUTICS, INC., Philadelphia, PA (US)

(72) Inventors: Xavier ANGUELA, Barcelona (ES); Sean ARMOUR, Holland, PA (US); Nicholas KEISER, Bryn Mawr, PA (US); Suryanarayan SOMANATHAN, Shrewsbury, MA (US); Mustafa N. YAZICIOGLU, Broomall, PA (US)

(73) Assignee: SPARK THERAPEUTICS, INC., Philadelphia, PA (US)

(21) Appl. No.: 17/050,362

(22) PCT Filed: Apr. 26, 2019
(86) PCT No.: PCT/US2019/029487

§ 371 Date: Oct. 23, 2020

Related U.S. Application Data
(60) Provisional application No. 62/663,963, filed on Apr. 27, 2018.

(51) Int. Cl. C07K 14/005; C12N 7/00; C12N 15/86; C12N 15/113

(52) U.S. Cl. CPC C07K 14/005; C12N 7/00; C12N 15/86; C12N 15/113; C12N 2750/14122; A61K 48/00; C07K 2319/09; C12N 2750/14143; C12N 2750/14123; C12N 2310/14; C12N 2320/32; C12N 2750/14171

(57) ABSTRACT

The invention provides modified adeno-associated virus (AAV) capsid proteins. Modified AAV capsid proteins include, for example, capsid proteins modified to have a peptide insertion comprising a nuclear localization signal (NLS) sequence, capsid proteins modified to have an amino acid substitution at an RXXL site or a (L/P)PXY site, where X can be any amino acid, and capsid proteins modified to have one or more particular amino acid positions substituted with a different amino acid.

### RELATED APPLICATIONS

This application claims priority to U.S. Provisional Patent Application No. 62/663,963, filed Apr. 27, 2018. The entire contents of the foregoing application is incorporated herein by reference, including all text, tables, sequence listing and drawings.

### INTRODUCTION

The capsid protein of adeno-associated virus (AAV) has five basic regions that may act as potential nuclear localization signals (NLS). These basic regions are named BR1, BR2, BR3, BR4 and BR5 (Grieger J C et al., 2006, J. Virol, 80:5199-5210; Liu P et al., 2017, Virol. J., 14:80, doi 10.1186/s12985-017-0745-1; Popa-Wagner R et al., 2012, J. Virol., 86:9163-9174). These basic regions are essential for virion assembly and/or viral infectivity. Due to its position on the capsid sequence, BR1 is unique to VP1; BR2 and BR3 are present on both VP1 and VP2; and BR4 and BR5 are present on VP1, VP2 and VP3. It has been reported that only a small fraction of recombinant adeno-associated virus (rAAV) particles (˜17%) can interact with the nuclear pore complex, the gateways into the nucleus, and are allowed to enter the nucleus (Kelich J M et al., 2015, Mol Ther Methods Clin Dev., 2:15047, doi.org/10.1038/mtm.2015.47).

### SUMMARY

To overcome the restrictive barrier for adeno-associated virus (AAV) transduction, a capsid modification strategy was used to increase AAV vector transduction of cells. Two approaches were used: 1) substituting certain amino acids into different regions of the AAV capsid proteins; and 2) inserting different types of peptides into various locations of the AAV capsid proteins. It was discovered that among different AAV capsid serotypes, certain point mutations of the AAV capsids and insertions of certain peptides into different regions of the AAV capsids can increase AAV vector transduction of cells.

In one embodiment, an AAV capsid protein is modified to have a peptide insertion, said peptide insertion comprising a nuclear targeting or localization signal (NLS). In particular aspects, a peptide insertion and/or nuclear localization sequence has a length from about 5 to about 60 amino acids.

In another embodiment, the nuclear localization sequence does not comprise an AAV nuclear localization sequence.

In another embodiment, the nuclear localization sequence comprises an additional AAV nuclear localization sequence from the same or different AAV serotype.

In another embodiment, an AAV capsid protein is modified to have an amino acid substitution at an RXXL site or a (L/P)PXY site, where X can be any amino acid.

In another embodiment, one or more lysine (K) residues substituted with arginine (R) residues. In particular aspects, a K→R substitution is at an RXXL site or a (L/P)PXY site, where X can be any amino acid. In further particular aspects, a K→R substitution is not at an RXXL site or a (L/P)PXY site, where X can be any amino acid.

In a further embodiment, an AAV capsid protein comprises an amino acid sequence having an alanine at position 517 of SEQ ID NO:1, an alanine at position 519 of SEQ ID NO:59 or 122 or an alanine at the corresponding position in the capsid protein of another AAV serotype.

In a further embodiment, an AAV capsid protein comprises an amino acid sequence at least 90% identical to SEQ ID NO:1, 59 or 122 and has an alanine at position 517 of SEQ ID NO:1, an alanine at position 519 of SEQ ID NO:59 or 122 or an alanine at the corresponding position in the capsid protein of another AAV serotype.

In a further embodiment, and AAV capsid protein comprises an amino acid sequence at least 90% identical to SEQ ID NO:1 and has an arginine at any of amino acid positions 137, 528, 533 and 545 of SEQ ID NO:1, or comprises an amino acid sequence at least 90% identical to a capsid protein of another AAV serotype and has an arginine at any of the corresponding amino acid positions in the capsid protein of the other AAV serotype.

In a further embodiment, and AAV capsid protein comprises an amino acid sequence at least 90% identical to SEQ ID NO:59 and has an arginine at any of amino acid positions 137, 333 and 530 of SEQ ID NO:59, or comprises an amino acid sequence at least 90% identical to a capsid protein of another AAV serotype and has an arginine at any of the corresponding amino acid positions in the capsid protein of the other AAV serotype.

In particular aspects, the peptide insertion is located at a position between basic region 1 (BR1) and basic region 2 (BR2) of the AAV capsid protein.

In particular aspects, the peptide insertion is located at a position between basic region 2 (BR2) and basic region 3 (BR3) of said AAV capsid protein.

In particular aspects, the peptide insertion is not located within basic region 1 (BR1), and/or basic region 2 (BR2), and/or basic region 3 (BR3), and/or basic region 4 (BR4) and/or basic region 5 (BR5) of said AAV capsid protein.

In particular aspects, the peptide insertion is in AAV VP1 and/or VP2 capsid proteins. In particular aspects, a peptide insertion is not in AAV VP2 capsid protein. In particular aspects, a peptide insertion is not in AAV VP3 capsid protein. In particular aspects, a peptide insertion is not in AAV VP3 capsid protein.

In particular aspects, the peptide insertion is not in basic region 1 (BR1), basic region 2 (BR2), basic region 3 (BR3), basic region 4 (BR4) or basic region 5 (BR5).

In particular aspects, the peptide insertion is not in is not in a phospholipase A2 (PLA2) domain.

In particular aspects, the peptide insertion is located in loop 3 (aka subloop I in loop IV) of VP1 capsid protein.

In particular aspects, the peptide insertion is located in amino acid positions 30-40, 135-141, 147-166, 380-390, 445-460 or 585-595 of VP1 capsid protein.

In particular aspects, the peptide insertion is located in amino acid positions 32-33, 34-35, 36-37, 138-139, 139-140, 162-163, 384-385, 450-451, 456-457, 588-589 or 590-591 of VP1 capsid protein.

In particular aspects, a modified AAV capsid protein has 90% or more sequence identity to a sequence selected from SEQ ID NOs:2-58.

In particular aspects, a modified AAV capsid protein has 90% or more sequence identity to a sequence selected from SEQ ID NOs:60-83.

In particular aspects, the peptide insertion is 90% or more identical to a sequence selected from SEQ ID NOs:84-104.

In particular aspects, the peptide insertion comprises at least two sequences selected from any of SEQ ID NOs:84-92 and 95.

In particular aspects, at least two of the peptide insertions are sequences selected from any of SEQ ID NOs:84-92 and 95 are the same sequence.

In particular aspects, at least two of the peptide insertions are sequences selected from any of SEQ ID NOs:84-92 and 95 are different sequences.

In particular aspects, the peptide insertion comprises a tandem repeat of a sequence selected from any of SEQ ID NOs:84-92 and 95.

In particular aspects, the peptide insertion comprises a tandem repeat of at least 2 sequences selected from any of SEQ ID NOs:84-92 and 95 wherein any of the 1^{st} of said at least 2 sequences is positioned at the 5 prime end of the 2^{nd} sequence and any of the 2^{nd} of said at least 2 sequences is positioned at the 3 prime end of the 1^{st} sequence.

In particular aspects, the peptide insertion comprises a tandem repeat of at least 3 sequences selected from any of SEQ ID NOs:93, 94 and 96-104.

In particular aspects, the peptide insertion comprises a tandem repeat of at least 3 sequences selected from any of SEQ ID NOs:84-92 and 95, wherein the 1^{st} of said at least 3 sequences is positioned at the 5 prime end of the 2^{nd} sequence, the 2^{nd} of said at least 3 sequences is positioned at the 3 prime end of the 1^{st} sequence and the 3^{rd} of said at least 3 sequences is positioned at the 3 prime end of the 2^{nd} sequence.

In particular aspects of at least two peptide insertion sequences, the at least two sequences are separated by 1, 2, 3, 4 or 5 intervening amino acid residues.

In various aspects where there are three or more peptide insertion sequences, and there are intervening amino acid residues between the insertion sequences, the intervening amino acid residues may or may not be not the same as each other.

In particular aspects, the peptide insertion comprises any of SEQ ID NOs:84-104 with one or more amino acid substitutions.

In particular aspects, the peptide insertion comprises any of SEQ ID NOs:84-104 with 1-10 amino acid substitutions.

In particular aspects, the peptide insertion comprises any of SEQ ID NOs:84-104 with one or more conservative amino acid substitutions.

In particular aspects, the parental AAV capsid protein is SEQ ID NO:1, SEQ ID NO:59 or AAV2.

In particular aspects, the parental AAV capsid protein comprises a VP1, VP2 and/or VP3 capsid sequence having 90% or more identity to Spk100 (SEQ ID NO:59), Spk200 (SEQ ID NO:1), AAV2 (SEQ ID NO:122), AAV1, AAV3, AAV4, AAV5, AAV6, AAV7, AAV8, AAV9, AAV10, AAV11, AAV12, Rh10, or Rh74 VP1, VP2 and/or VP3 sequences.

The invention also provides recombinant adeno-associated virus (rAAV) particles comprising: a modified AAV capsid protein as set forth herein; and a vector genome comprising a heterologous nucleic acid sequence.

The invention further provides recombinant adeno-associated virus (rAAV) particles comprising a modified AAV capsid protein as set forth herein, wherein some or all of the rAAV particles are devoid of a heterologous nucleic acid sequence. In particular aspects, such AAV particles have not packaged a full-length heterologous nucleic acid sequence.

In particular embodiments, the peptide insertion increases or enhances entry or transduction of the invention rAAV particle into the nucleus of a cell, as compared to entry or transduction into the nucleus of a cell of an rAAV particle comprising the parental AAV capsid protein.

In particular embodiments, the peptide insertion increases or enhances escape of the rAAV particle from cell endosomes as compared to escape of an rAAV particle comprising the parental AAV capsid protein from cell endosomes.

In particular embodiments, the peptide insertion reduces or decreases degradation of said rAAV particle in cells as compared to degradation in cells of an rAAV particle comprising said parental AAV capsid protein.

The invention also provides pharmaceutical compositions comprising rAAV particles that comprise the modified AAV capsid proteins as set forth herein and a heterologous nucleic acid sequence.

The invention further provides pharmaceutical compositions that include AAV empty capsids that comprise the modified AAV proteins, wherein the rAAV empty capsid proteins are devoid of a heterologous nucleic acid sequence.

The invention moreover provides pharmaceutical compositions that comprise mixtures of rAAV particles that comprise the modified AAV capsid proteins as set forth herein and a heterologous nucleic acid sequence and rAAV empty capsids that comprise the modified/variant AAV capsid proteins, wherein the rAAV empty capsids are devoid of a heterologous nucleic acid sequence.

The invention additionally provides methods for delivering or transferring a heterologous nucleic acid sequence into a mammal or a cell of a mammal, comprising administering the rAAV particles of the invention, including pharmaceutical compositions comprising the rAAV particles of the invention.

In one embodiment, a method includes treating a mammal deficient in protein expression or function, by administering an effective amount of an invention rAAV particle or pharmaceutical composition thereof to the mammal.

In one aspect, the heterologous nucleic acid sequence encodes a protein having a function of the deficient protein, and wherein said protein having said function of said deficient protein is expressed in said mammal, thereby treating said mammal deficient in protein expression or function.

In a particular aspect, a heterologous nucleic acid sequence encodes a blood coagulation Factor. In more particular aspects, a heterologous nucleic acid sequence encodes Factor VII, VIII, IX, X, XI, V, XII, II, von Willebrand factor, vitamin K epoxide reductase C1, or gamma-carboxylase.

In further particular aspects, the Factor VIII has a B domain deletion (BDD). In still further particular aspects, the heterologous nucleic acid encoding Factor VIII with the B domain deletion has 20 or fewer, 15 or fewer, or 10 or fewer cytosine-guanine dinucleotides (CpGs), for example, no more than 5 cytosine-guanine dinucleotides (CpGs), such as only 4, 3, 2, 1 or 0 cytosine-guanine dinucleotides (CpGs).

In additional particular aspects, the Factor VIII comprises SEQ ID NO:123 having a deletion of one or more amino acids of the sequence SFSQNPPVLKRHQR (SEQ ID NO:124), or a deletion of the entire sequence SFSQNPPVLKRHQR.

In particular aspects, the rAAV particle comprises a heterologous nucleic acid sequence that encodes acid alpha-glucosidase (GAA); ATP7B (copper transporting ATPase2); alpha galactosidase; ASS1 (arginosuccinate synthase); beta-glucocerebrosidase; beta-hexosaminidase A; SERPING1 (C1 protease inhibitor); glucose-6-phosphatase; erythropoietin (EPO; interferon-alpha; interferon-beta; interferon-gamma; an interleukin (IL); any one of Interleukins 1-36 (IL-1 through IL-36); interleukin (IL) receptor; a chemokine; chemokine (C—X—C motif) ligand 5 (CXCL5); granulocyte-colony stimulating factor (G-CSF); granulocyte-macrophage colony stimulating factor (GM-CSF); macrophage colony stimulating factor (M-CSF); keratinocyte growth factor (KGF); monocyte chemoattractant protein-1 (MCP-1); tumor necrosis factor (TNF); a tumor necrosis factor (TNF) receptor; alpha-1 antitrypsin; alpha-L-iduronidase; ornithine transcarbamoylase; phenylalanine hydroxylase (PAH); phenylalanine ammonia-lyase (PAL); lipoprotein lipase; an apolipoprotein; low-density lipoprotein receptor (LDL-R); albumin; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein, T-protein, cystic fibrosis transmembrane regulator (CFTR); ATP-binding cassette, sub-family A (ABC1), member 4 (ABCA4); or dystrophin.

In particular aspects, the rAAV particle comprises a heterologous nucleic acid sequence that encodes an inhibitory RNA. In more particular aspects, the inhibitory RNA comprises a short hairpin (sh)RNA, a microRNA (miRNA), a small or short interfering (si)RNA, a trans-splicing RNA, or an antisense RNA.

Subjects administered invention rAAV vectors in accordance with the invention include mammals, such as humans. Particular subjects are those in need of administration.

In various embodiments, the human has a blood clotting disorder, such as hemophilia A or hemophilia B, Pompe disease, Wilson's disease, Fabry disease, citrullinemia type 1, Gaucher disease type 1, Tay Sachs disease, hereditary angioedema (HAE), glycogen storage disease type I (GSDI), anemia, an interferon-alpha, interferon-beta, or interferon-gamma related immune disorder, a viral infection, cancer, an inflammatory disease, an immune deficiency, an immune disorder, Crohn's disease, epithelial tissue damage, insulin resistance, emphysema, chronic obstructive pulmonary disease (COPD), mucopolysaccharidosis I (MPS I), ornithine transcarbamylase (OTC) deficiency, phenylketonuria (PKU), lipoprotein lipase deficiency, apolipoprotein (Apo) A-I deficiency, familial hypercholesterolemia (FH), Stargardt disease or hypoalbuminemia.

The invention also provides methods of producing invention rAAV particles. In one embodiment, a method includes introducing a nucleic acid encoding the modified AAV capsid protein as set forth herein into a packaging helper cell, in which the helper cell comprises an AAV vector genome; and culturing the helper cell under conditions to produce the rAAV particle. In another embodiment a method includes introducing a nucleic acid encoding the modified AAV capsid protein as set forth herein and introducing the AAV vector genome into a packaging helper cell; and culturing the helper cells under conditions to produce the rAAV particle.

Helper cells comprise mammalian cells. Helper cells provide helper functions that package the AAV vector genome into an AAV particle. Examples of helper functions include Rep and/or Cap protein sequence(s), such as Rep78 or/and Rep68 proteins. Such cells may be stably or transiently transfected with Rep78 and Rep68 proteins polynucleotide encoding sequence(s). Such cells include, for example, HEK-293 cells.

### DESCRIPTION OF DRAWINGS

FIG. 1A shows luciferase activity in lysates of Huh7 cells transduced with Spark200, Spark200-140cmycNLS, Spark200-140class3NLS, Spark200-140hnRNP_D_NLS and Spark200-L517A vectors, carrying a Renilla luciferase transgene.

FIG. 1B shows luciferase activity in lysates of HEK293 cells transduced with Spark200, Spark200-140cmycNLS, Spark200-140class3NLS, Spark200-140hnRNP_D_NLS and Spark200-L517A vectors, carrying a Renilla luciferase transgene.

FIG. 1C shows relative amounts of FIX in media of cultures of Huh7 cells transduced (20,000 MOI) with Spark200 (100%), Spark200-140cmycNLS, Spark200-140class3NLS, Spark200-140hnRNP_D_NLS and Spark200-L517A rAAV vectors, carrying a FIX transgene.

FIG. 2 shows luciferase activity in lysates of Huh7 cells transduced (2,000 MOI) with Spark200 rAAV or various Spark200 variant rAAV having cell penetrating peptide inserts, carrying a Renilla luciferase transgene. All the cell penetrating peptides decreased the transduction as compared to Spark200. Moreover, some of the peptide inserts decreased the vector production yield, making them difficult to produce.

FIG. 3 shows relative amounts of luciferase activity in Huh7 cells following transduction by Spark100 variant rAAVs with 1×NLS insertions, carrying a Renilla luciferase transgene, compared to wild type Spark100 rAAV (100%).

FIG. 4 shows relative amounts of luciferase activity in Huh7 cells following transduction by Spark100 variant rAAVs with 2×NLS insertions, carrying a Renilla luciferase transgene, compared to wild type Spark100 rAAV (100%).

FIG. 5 shows relative amounts of luciferase activity in Huh7 cells following transduction by Spark100 variant rAAVs with 3×NLS inserts, carrying a Renilla luciferase transgene, compared to wild type Spark100 rAAV (100%).

FIG. 6 shows the location of peptide insertions for Spark100 and Spark200 in the VP1, VP2 and VP3 capsid proteins.

FIG. 7 shows relative amounts of luciferase activity in Huh7 cells following transduction by Spark100 variant rAAVs with insertions into the region between BR2 and BR3 compared to wild type Spark100 rAAV (100%).

FIG. 8 shows a graph of FIX levels (ng/mL) in the plasma of C57BL/6 mice, measured by ELISA, at various timepoints following administration of (1) wild type AAV2 rAAV particles carrying a FIX transgene (circles) or (2) AAV2 rAAV particles with cmycNLS (SEQ ID NO:86) inserted into VP1 (between amino acid residues 34 and 35), also carrying a FIX transgene (squares).

FIG. 9 shows an alignment of AAV2 (SEQ ID NO:122), Spk200 (SEQ ID NO:1) and Spk100 (SEQ ID NO: 59) VP1 capsid proteins, representative insertion sites, the phospholipase A2 (PLA2) domain and the 5 basic regions (BRs).

FIG. 10 shows GAA activity in the plasma of C57BL/6 mice, three weeks after administration of rAAV particles with wild type Spark100 or Spark100 variant capsids having SV40 and class3 NLS insertions, and carrying a GAA transgene.

FIG. 11 shows GAA activity in the plasma of C57BL/6 mice, one week after administration of rAAV particles with wild type Spark100 capsid or with variant capsids having NLS peptide insertions: Spark100-140-cmycNLS (labeled SparkX04), Spark100-140-hnRNP_D_NLS (labeled SparkX05), Spark100-140-class3 NLS (labeled SparkX06) and Spark100-140-2×cmycNLS (labeled SparkX08).

FIGS. 12A and 12B show luciferase activity in lysates of Huh7 cells transduced with Spark100 rAAV or various Spark100 variant rAAV having NLS peptide insertions in only VP1, carrying a Renilla luciferase transgene. The variants were as follows: Spark100-33-cmycNLS (labeled asSpark100_X42), Spark100-35-cmycNLS (labeled as Spark100_X43), Spark100-33-2×cmycNLS (labeled as Spark100_X44), Spark100-35-2×cmycNLS (labeled as Spark100_X45), Spark100-35-3×cmycNLS (labeled as Spark100_X46), Spark100-35-1×Class3NLS (labeled as Spark100_X47), Spark100-35-2×Class3NLS (labeled as Spark100_X48, Spark100-35-3×Class3NLS (labeled as Spark100_X49), Spark100-35-hnRNP_NLS (labeled as Spark100_X50), and Spark100-35-1×-SV40 NLS (labeled as Spark100_X51).

FIG. 13 shows luciferase activity in lysates of Huh7 cells transduced with Spark100 rAAV orSpark100 having K4R point mutations at positions 333 (K333R; labeled Spark100_X52) and 530 (K530R; labeled Spark100_X53).

FIG. 14 shows luciferase activity in lysates of Huh7 cells transduced with Spark100 rAAV or the following Spark100 variants having VP3 region NLS insertions (insertion is in all of VP1, VP2 and VP3 proteins): Spark100_385cmycNLS (labeled as Spark100_X54), Spark100_451cmycNLS (labeled as Spark100_X55), Spark100_457cmycNLS (labeled as Spark100_X56), Spark100_589cmycNLS (labeled as Spark100_X57) and Spark100_591cmycNLS (labeled as Spark100-X58).

FIG. 15 shows an alignment of the amino acid sequences for AAV1-AAV11, AAV3B, Spk200 (SEQ ID NO:1) and Spk100 (SEQ ID NO:59) VP1 capsid proteins.

FIGS. 16A-16F show representative insertion sites in AAV1-AAV11, AAV3B, Spk200 and Spk100 VP1 capsid proteins.

### DETAILED DESCRIPTION

The invention provides modified AAV capsid proteins, nucleic acids encoding modified AAV capsid proteins, AAV vector genomes comprising nucleic acids encoding modified AAV proteins, recombinant AAV vectors and particles comprising the modified AAV proteins of the invention.

As used herein, the terms “modify” or “variant” and grammatical variations thereof, mean that a nucleic acid or protein deviates from a reference or parental sequence. A modified or variant protein refers to a protein sequence which has been altered compared to reference (e.g., wild-type) or parental sequence. Modified and variant sequences may therefore have substantially the same, greater or less activity or function than a reference or parental sequence, but at least retain partial activity or function of the reference or parental sequence. The sequence may be genetically modified to encode a modified or variant protein.

A “modified AAV capsid protein” or “variant AAV capsid protein” means that the capsid protein has an amino acid alteration compared the parental unmodified AAV capsid protein. Such an AAV capsid protein can be referred to as a modified or variant AAV capsid protein. A particular example of a modification of a capsid protein is an amino acid substitution. Another particular example of a modification of a capsid protein is a peptide insertion. The terms “modification” or “variant” herein need not appear in each instance of a reference made to an AAV capsid protein.

In particular embodiments, a modified or variant capsid protein retains at least part of a function or activity of unmodified reference or parental capsid protein. The function or activity of an AAV capsid protein includes the ability to package the AAV vector genome into productive viral particles that are able to transduce cells and in turn able introduce the heterologous sequence in the vector genome in the transduced cells for expression. Such an AAV capsid protein that has been modified as set forth herein can be compared to the unmodified reference or parental AAV capsid protein.

Accordingly, the AAV capsid “proteins,” “polypeptides” and “peptides” include modified forms or variants so long as the modified form or variant retains some degree or aspect of functionality of the reference or parental AAV capsid. For example, a modified AAV capsid protein may retain the ability to package the AAV vector genome into productive AAV particles which subsequently can be used to transduce cells thereby introducing a heterologous nucleic acid sequence into the cells for subsequent suppression.

In addition, a modified/variant AAV capsid protein may not retain the ability to package an AAV vector genome but still be able to form AAV particles or in the case of a modified AAV capsid protein not containing a vector genome but again is still able to form AAV particles, such modified AAV capsid proteins are considered functional since they form AAV particles which may be used as decoys to bind to neutralizing AAV antibodies that may be present in subjects that have pre-existing AAV neutralizing antibodies or are anticipated to develop AAV neutralizing antibodies against AAV particles, for example, as a result of AAV-based gene therapy. Such AAV particles that are unable to package an AAV vector genome or do not have an AAV vector genome comprising a heterologous nucleic acid sequence are still useful in the context of gene therapy since such AAV particles can be used to block AAV neutralizing antibodies from binding to AAV particles that have packaged an AAV vector genome that comprises a heterologous nucleic sequence.

As set forth herein, modified/variant AAV capsid proteins can exhibit different characteristics or have improvements compared to a reference or parental AAV capsid protein. When comparing the different characteristics or improvements, it is appropriate to compare it to the reference or parental AAV capsid protein. In the case that the parental AAV capsid protein has already been modified, a further modification of the parental AAV capsid protein as set forth herein, for example an amino acid substitution or peptide insertion as set forth herein, the comparison is to the parental modified AAV capsid protein prior to the amino acid substitution or peptide insertion.

A “nuclear localization signal” (NLS) peptide is typically a short peptide sequence that can mediate or facilitate nuclear import (passage into the cellular nucleus) of molecules such as proteins by binding to NLS receptors, known as importins or karyopherins.

Although not wishing to be bound by any theory, it is believed that the NLS peptide insertions operate by increasing or enhancing nuclear transduction of the recombinant AAV vector. Increased or enhanced “nuclear transduction” refers to increased tropism for the nucleus, which includes, for example, increased localization into the cell nucleus, increased endocytosis by the cell nucleus, increased penetration of the cell nucleus, increased entry into the cell nucleus, increased importation into the nucleus, increased targeting of the cell nucleus, increased binding to the cell nucleus and grammatical variations thereof.

Nuclear transduction could occur through many mechanisms. For example, the NLS peptide insertion could bind to a protein within the cell which functions as a carrier to transport a recombinant AAV into the nucleus. Alternatively, a recombinant AAV could bind to a target on the nuclear membrane which is subsequently internalized within the cell nucleus.

A peptide insertion may provide the recombinant AAV with increased binding affinity for the surface of the cell membrane, due for example to increased affinity for a cell surface protein. This increased binding affinity could lead to increased endocytosis into the cytoplasm and subsequent transduction into the cell's nucleus. A particular example of such a peptide insertion is a cell penetrating peptide which can bind to the cell membrane and be internalized within the cell. A “cell penetrating peptide” refers to a peptide sequence capable of crossing the cell membrane and that can mediate or facilitate entry (passage) through the cell membrane of other molecules, including, for example, proteins, nucleic acids, therapeutics and nanoparticles. After internalization, the recombinant AAV subsequently enters into the nucleus, for example, by fusion of cell membrane components to the nuclear membrane.

Peptide insertions as set forth herein may provide additional characteristics to the recombinant AAV. It is expressly intended that any other characteristics provided by the NLS peptide insertions or cell penetrating peptide insertions are not excluded by any potential mechanism for increased transduction into the nucleus of a cell or increased transduction into the cell, respectively, as set forth herein.

As set forth herein AAV capsid modifications/variants include amino acid substitutions and peptide insertions. Non-limiting examples of amino acid substitutions include substituting 1-3, 3-5, 5-10, 10-15, 15-20, 20-25, 25-30, 30-40, 40-50, 50-100, 100-150, 150-200, 200-250 or more amino acid residues. Non-limiting examples of peptide insertions include 2 or more contiguous/adjacent amino acid residues inserted into AAV capsids, for example, 2-3, 3-5, 5-10, 10-15, 15-20, 20-25, 25-30, 30-40, 40-50, 50-100 or more amino acid residues. In the situation of a substitution as set forth herein, where there are 2 or more substitutions made, the substitutions are not adjacent to each other. In other words, if there are 2 or more amino acid substitutions and the 2 or more amino acid residues are contiguous/adjacent to each other than they are considered a peptide insertion.

In certain embodiments, a peptide insertion has a length of about 5 amino acids to about 60 amino acids. In further embodiments, a peptide insertion has a length of about 8 amino acids to about 50 amino acids. In additional embodiments, a peptide insertion has a length of about 10 amino acids to about 40 amino acids. In still further embodiments, a peptide insertion has a length of about 10 amino acids to about 30 amino acids. In still additional embodiments, a peptide insertion has a length of about 10 amino acids to about 25 amino acids.

The term “vector” refers to small carrier nucleic acid molecule, a plasmid, virus (e.g., AAV vector), or other vehicle that can be manipulated by insertion or incorporation of a nucleic acid. Such vectors can be used for genetic manipulation (i.e., “cloning vectors”), to introduce/transfer polynucleotides into cells, and to transcribe or translate the inserted polynucleotide in cells. An “expression vector” is a specialized vector that contains a gene or nucleic acid sequence with the necessary regulatory regions needed for expression in a host cell. A vector nucleic acid sequence generally contains at least an origin of replication for propagation in a cell and optionally additional elements, such as a heterologous polynucleotide sequence, expression control element (e.g., a promoter, enhancer), intron, an inverted terminal repeat (ITR), selectable marker (e.g., antibiotic resistance), polyadenylation signal.

A viral vector is derived from or based upon one or more nucleic acid elements that comprise a viral genome. A particular viral vector is an adeno-associated virus (AAV) vector.

The term “recombinant,” as a modifier of vector, such as recombinant AAV vector, as well as a modifier of sequences such as recombinant polynucleotides and polypeptides, means that the compositions have been manipulated (i.e., engineered) in a fashion that generally does not occur in nature. A particular example of a recombinant AAV vector would be where a click acid sequence that is not normally present in the wild-type AAV genome is inserted within the AAV genome. Although the term “recombinant” is not always used herein in reference to AAV vectors, as well as sequences such as polynucleotides, recombinant forms including polynucleotides, are expressly included in spite of any such omission.

A “recombinant AAV vector” or “rAAV” is derived from the wild type (wt or wild-type) genome of AAV by using molecular methods to remove the wild type genome from the AAV genome, and replacing with a non-native nucleic acid sequence, referred to as a heterologous nucleic acid. Typically, for AAV one or both inverted terminal repeat (ITR) sequences of AAV genome are retained in the AAV vector. rAAV is distinguished from an AAV genome, since all or a part of the AAV genome has been replaced with a non-native sequence with respect to the AAV genomic nucleic acid. Incorporation of a non-native sequence therefore defines the AAV vector as a “recombinant” vector, which can be referred to as a “rAAV vector.”

A rAAV sequence can be packaged—referred to herein as a “particle”—for subsequent infection (transduction) of a cell, ex vivo, in vitro or in vivo. Where a recombinant AAV vector sequence is encapsidated or packaged into an AAV particle, the particle can also be referred to as a “rAAV vector” or “rAAV particle.” Such rAAV particles include proteins that encapsidate or package the vector genome. In the case of AAV, they are referred to as capsid proteins.

A vector “genome” refers to the portion of the recombinant plasmid sequence that is ultimately packaged or encapsidated to form a viral (e.g., AAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsidated into virus (e.g., AAV) particles. Thus, a vector “genome” refers to the nucleic acid that is packaged or encapsidated by virus (e.g., AAV).

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids include naturally occurring, synthetic, and intentionally modified or altered polynucleotides (e.g., variant nucleic acid). The nucleic acids such as cDNA, genomic DNA, RNA, and fragments thereof which may be single- or double-stranded.

Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

A “transgene” is used herein to conveniently refer to a heterologous nucleic acid that is intended or has been introduced into a cell or organism. Transgenes include any heterologous nucleic acid, such as a gene that encodes a polypeptide or protein or encodes an inhibitory RNA.

A heterologous nucleic acid can be introduced/transferred by way of vector, such as AAV, “transduction” or “transfection” into a cell. The term “transduce” and grammatical variations thereof refer to introduction of a molecule such as an rAAV vector into a cell or host organism. The heterologous nucleic acid/transgene may or may not be integrated into genomic nucleic acid of the recipient cell. The introduced heterologous nucleic acid may also exist in the recipient cell or host organism extrachromosomally, or only transiently.

A “transduced cell” is a cell into which the transgene has been introduced. Accordingly, a “transduced” cell (e.g., in a mammal, such as a cell or tissue or organ cell), means a genetic change in a cell following incorporation of an exogenous molecule, for example, a nucleic acid (e.g., a transgene) into the cell. Thus, a “transduced” cell is a cell into which, or a progeny thereof in which an exogenous nucleic acid has been introduced. The cell(s) can be propagated and the introduced protein expressed, or nucleic acid transcribed. For gene therapy uses and methods, a transduced cell can be in a subject.

An “expression control element” refers to nucleic acid sequence(s) that influence expression of an operably linked nucleic acid. Control elements, including expression control elements as set forth herein such as promoters and enhancers. Vector sequences including AAV vectors can include one or more “expression control elements.” Typically, such elements are included to facilitate proper heterologous polynucleotide transcription and if appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of AAV vectors, expression control elements will typically be within 1 to 1000 nucleotides from the transcribed nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed nucleic acid sequence. A promoter typically increases an amount expressed from operably linked nucleic acid as compared to an amount expressed when no promoter exists.

An “enhancer” as used herein can refer to a sequence that is located adjacent to the heterologous nucleic acid. Enhancer elements are typically located upstream of a promoter element but also function and can be located downstream of or within a sequence. Hence, an enhancer element can be located 10-50 base pairs, 50-100 base pairs, 100-200 base pairs, or 200-300 base pairs, or more base pairs upstream or downstream of a heterologous nucleic acid sequence. Enhancer elements typically increase expressed of an operably linked nucleic acid above expression afforded by a promoter element.

An expression construct may comprise regulatory elements which serve to drive expression in a particular cell or tissue type. Expression control elements (e.g., promoters) include those active in a particular tissue or cell type, referred to herein as a “tissue-specific expression control elements/promoters.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Sambrook et al. (1989) and Ausubel et al. (1992)).

The incorporation of tissue specific regulatory elements in the expression constructs provides for at least partial tissue tropism for the expression of a heterologous nucleic acid encoding a protein or inhibitory RNA. Examples of promoters that are active in liver are the transthyretin (TTR) promoter (SEQ ID NO:125); mutant TTR promoter (SEQ ID NO:126); human alpha 1-antitrypsin (hAAT) promoter; albumin promoter, Miyatake, et al., J. Virol., 71:5124-32 (1997); hepatitis B virus core promoter, Sandig, et al., Gene Ther. 3:1002-9 (1996); and the alpha-fetoprotein (AFP) promoter, Arbuthnot, et al., Hum. Gene. Ther., 7:1503-14 (1996), among others. An example of an enhancer active in liver is apolipoprotein E (apoE) HCR-1 and HCR-2 (Allan et al., J. Biol. Chem., 272:29113-19 (1997)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al., Cell, 41:521-530 (1985)), the SV40 promoter, the dihydrofolate reductase promoter, the cytoplasmic β-actin promoter and the phosphoglycerol kinase (PGK) promoter.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268:1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J. Clin. Invest. 100:2865-2872 (1997); Rivera, et al., Nat. Medicine. 2:1028-1032 (1996)). Other regulatable control elements which may be useful in this context are those which are regulated by a specific physiological state, e.g., temperature, acute phase, development.

Expression control elements also include the native elements(s) for the heterologous polynucleotide. A native control element (e.g., promoter) may be used when it is desired that expression of the heterologous polynucleotide should mimic the native expression. The native element may be used when expression of the heterologous polynucleotide is to be regulated temporally or developmentally, or in a tissue-specific manner, or in response to specific transcriptional stimuli. Other native expression control elements, such as introns, polyadenylation sites or Kozak consensus sequences may also be used.

The term “operably linked” means that the regulatory sequences necessary for expression of a nucleic acid sequence are placed in the appropriate positions relative to the sequence so as to effect expression of the nucleic acid sequence. This same definition is sometimes applied to the arrangement of nucleic acid sequences and transcription control elements (e.g. promoters, enhancers, and termination elements) in an expression vector, e.g., rAAV vector.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. For a nucleic acid sequence less than 4.7 kb, the filler or stuffer polynucleotide sequence has a length that when combined (e.g., inserted into a vector) with the sequence has a total length between about 3.0-5.5 kb, or between about 4.0-5.0 kb, or between about 4.3-4.8 kb.

Where a wild type heterologous nucleic acid or transgene is too large to be packaged within an AAV vector particle, the heterologous nucleic acid may be provided in modified, fragmented or truncated form for packaging in and delivery by an AAV vector, such that a functional protein or nucleic acid product, such as a therapeutic protein or nucleic acid product, is ultimately provided.

In some embodiments, the heterologous nucleic acid that encodes a protein (e.g., therapeutic protein) is provided in modified or truncated forms or the heterologous nucleic acid is provided in multiple constructs, delivered by separate and multiple AAV vectors.

In certain aspects, the heterologous nucleic acid is provided as a truncated variant that maintains functionality of the encoded protein (e.g., therapeutic protein), including removal of portions unnecessary for function, such that the encoding heterologous polynucleotide is reduced in size for packaging in an AAV vector.

In certain aspects the heterologous nucleic acid is provided in split AAV vectors, each providing nucleic acid encoding different portions of a protein (e.g., therapeutic protein), thus delivering multiple portions of a protein (e.g., therapeutic protein) which assemble and function in the cell.

In other aspects, the heterologous nucleic acid is provided by dual AAV vectors using overlapping, trans-splicing or hybrid trans-splicing dual vector technology. In certain embodiments, two overlapping AAV vectors are used which combine in the cell to generate a full expression cassette, from which a full-length protein (e.g., therapeutic protein) is expressed.

A “hemostasis related disorder” refers to bleeding disorders such as hemophilia A, hemophilia A with inhibitory antibodies, hemophilia B, hemophilia B with inhibitory antibodies, a deficiency in any coagulation Factor: VII, VIII, IX, X, XI, V, XII, II, von Willebrand factor, combined FV/FVIII deficiency, thalassemia, vitamin K epoxide reductase C1 deficiency, or gamma-carboxylase deficiency; bleeding associated with trauma, injury, thrombosis, thrombocytopenia, stroke, coagulopathy, or disseminated intravascular coagulation (DIC); over-anticoagulation associated with heparin, low molecular weight heparin, pentasaccharide, warfarin, or small molecule antithrombotics (i.e., FXa inhibitors); and platelet disorders such as, Bernard Soulier syndrome, Glanzmann thrombasthenia, and storage pool deficiency.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

The term “isolated” does not exclude combinations produced by the hand of man, for example, a rAAV sequence, or rAAV particle that packages or encapsidates an AAV vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or at least 85% by weight, or about 90-99% by weight, of the compound of interest. Purity is measured by methods appropriate for the compound of interest (e.g., chromatographic methods, agarose or polyacrylamide gel electrophoresis, HPLC analysis, and the like).

The phrase “consisting essentially of” when referring to a particular nucleotide sequence or amino acid sequence means a sequence having the properties of a given SEQ ID NO. For example, when used in reference to an amino acid sequence, the phrase includes the sequence per se and molecular modifications that would not affect the basic and novel characteristics of the sequence.

The term “identity,” “homology” and grammatical variations thereof, mean that two or more referenced entities are the same, when they are “aligned” sequences. Thus, by way of example, when two protein sequences are identical, they have the same amino acid sequence, at least within the referenced region or portion. Where two nucleic acid sequences are identical, they have the same nucleic acid sequence, at least within the referenced region or portion. The identity can be over a defined area (region or domain) of the sequence.

An “area” or “region” of identity refers to a portion of two or more referenced entities that are the same. Thus, where two protein or nucleic acid sequences are identical over one or more sequence areas or regions they share identity within that region. An “aligned” sequence refers to multiple protein (amino acid) or nucleic acid sequences, often containing corrections for missing or additional bases or amino acids (gaps) as compared to a reference sequence.

The identity can extend over the entire length or a portion of the sequence. In certain embodiments, the length of the sequence sharing the percent identity is 2, 3, 4, 5 or more contiguous amino acids or nucleic acids, e.g., 6, 7, 8, 9, 10, 11, 12, 13, 14, 15, 16, 17, 18, 19, 20, etc. contiguous nucleic acids or amino acids. In additional embodiments, the length of the sequence sharing identity is 21 or more contiguous amino acids or nucleic acids, e.g., 21, 22, 23, 24, 25, 26, 27, 28, 29, 30, 31, 32, 33, 34, 35, 36, 37, 38, 39, 40, etc. contiguous amino acids or nucleic acids. In further embodiments, the length of the sequence sharing identity is 41 or more contiguous amino acids or nucleic acids, e.g., 42, 43, 44, 45, 45, 47, 48, 49, 50, etc., contiguous amino acids or nucleic acids. In yet further embodiments, the length of the sequence sharing identity is 50 or more contiguous amino acids or nucleic acids, e.g., 50-55, 55-60, 60-65, 65-70, 70-75, 75-80, 80-85, 85-90, 90-95, 95-100, 100-150, 150-200, 200-250, 250-300, 300-500, 500-1,000, etc. contiguous amino acids or nucleic acids.

As set forth herein, modified/variant AAV capsids will be distinct from wild-type but may exhibit sequence identity with the parental or reference AAV capsid. Modified/variant AAV capsids will typically be at least about 70%-80% identical, more typically about 80%-90% identical, even more typically about 90-99.9% identical, and most typically 95%-99.9% identical to parental or reference AAV capsid protein.

The extent of identity (homology) or “percent identity” between two sequences can be ascertained using a computer program and/or mathematical algorithm. For purposes of this invention comparisons of nucleic acid sequences are performed using the GCG Wisconsin Package version 9.1, available from the Genetics Computer Group in Madison, Wis. For convenience, the default parameters (gap creation penalty=12, gap extension penalty=4) specified by that program are intended for use herein to compare sequence identity. Alternately, the Blastn 2.0 program provided by the National Center for Biotechnology Information (found on the world wide web at ncbi.nlm.nih.gov/blast/; Altschul et al., 1990, J Mol Biol 215:403-410) using a gapped alignment with default parameters, may be used to determine the level of identity and similarity between nucleic acid sequences and amino acid sequences. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Nucleic acid molecules, expression vectors (e.g., AAV vector genomes), plasmids, including nucleic acid encoding modified/variant AAV capsids of the invention and heterologous nucleic acids may be prepared by using recombinant DNA technology methods. The availability of nucleotide sequence information enables preparation of isolated nucleic acid molecules of the invention by a variety of means. For example, nucleic acid sequences encoding modified/variant AAV capsids can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Nucleic acids encoding modified/variant AAV capsids may be maintained as DNA in any convenient cloning vector. In a one embodiment, clones are maintained in a plasmid cloning/expression vector, such as pBluescript (Stratagene, La Jolla, Calif.), which is propagated in a suitable E. coli host cell. Alternatively, nucleic acids may be maintained in vector suitable for expression in mammalian cells, for example, an AAV vector. In cases where post-translational modification affects coagulation function, nucleic acid molecule can be expressed in mammalian cells.

As disclosed herein, rAAV vectors may optionally comprise regulatory elements necessary for expression of the heterologous nucleic acid in a cell positioned in such a manner as to permit expression of the encoded protein in the host cell. Such regulatory elements required for expression include, but are not limited to, promoter sequences, enhancer sequences and transcription initiation sequences as set forth herein and known to the skilled artisan.

Methods and uses of the invention include delivering (transducing) nucleic acid (transgene) into host cells, including dividing and/or non-dividing cells. The nucleic acids, rAAV vector, methods, uses and pharmaceutical formulations of the invention are additionally useful in a method of delivering, administering or providing sequence encoded by heterologous nucleic acid to a subject in need thereof, as a method of treatment. In this manner, the nucleic acid is transcribed and a protein or inhibitory nucleic acid may be produced in vivo in a subject. The subject may benefit from or be in need of the protein or inhibitory nucleic acid because the subject has a deficiency of the protein, or because production of the protein or inhibitory nucleic acid in the subject may impart some therapeutic effect, as a method of treatment or otherwise. For example, an inhibitory nucleic acid can reduce expression or transcription of an aberrant deleterious protein that is expressed in a subject in which the apparent or deleterious protein causes a disease or disorder, such as a neurological disease or disorder.

rAAV vectors comprising an AAV genome with a heterologous nucleic acid permit the treatment of genetic diseases, e.g., a protein or enzyme deficiency, such as a GAA or FVIII deficiency. For deficiency state diseases, gene transfer can be used to bring a normal gene into affected tissues for replacement therapy, as well as to create animal models for the disease using antisense mutations. For unbalanced disease states, gene transfer could be used to create a disease state in a model system, which could then be used in efforts to counteract the disease state. The use of site-specific integration of nucleic acid sequences to correct defects is also possible.

In various embodiments, rAAV vectors comprising aa AAV genome with a heterologous nucleic acid may be used, for example, as therapeutic and/or prophylactic agents (protein or nucleic acid). In particular embodiments, the heterologous nucleic acid encodes a protein that can modulate the blood coagulation cascade.

For example, an encoded FVIII or hFVIII-BDD may have similar coagulation activity as wild-type FVIII, or altered coagulation activity compared to wild-type FVII. Administration of FVIII or hFVIII-BDD-encoding rAAV vectors to a patient results in the expression of FVIII or hFVIII-BDD protein which serves to normalize the coagulation cascade.

In additional embodiments, a heterologous nucleic acid encodes a protein (enzyme) that can inhibit or reduce the accumulation of glycogen, prevent the accumulation of glycogen or degrade glycogen. For example, an encoded GAA may have similar activity as wild-type GAA. Administration of GAA-encoding rAAV vectors to a patient with Pompe disease results in the expression of the GAA protein which serves to inhibit or reduce the accumulation of glycogen, prevent the accumulation of glycogen or degrade glycogen, which in turn can reduce or decrease one or more adverse effects of Pompe disease.

Non-limiting examples of diseases treatable in accordance with the invention include lung disease (e.g., cystic fibrosis), a bleeding disorder (e.g., hemophilia A or hemophilia B with or without inhibitors), thalassemia, a blood disorder (e.g., anemia), Alzheimer's disease, Parkinson's disease, Huntington's disease, amyotrophic lateral sclerosis (ALS), epilepsy, a lysosomal storage disease) e.g., aspartylglucosaminuria, Batten disease, late infantile neuronal ceroid lipofuscinosis type 2 (CLN2), cystinosis, Fabry disease, Gaucher disease types I, II, and III, glycogen storage disease II (Pompe disease), ganglioside monosialic 2 (GM2)-gangliosidosis type I (Tay Sachs disease), GM2-gangliosidosis type II (Sandhoff disease), mucolipidosis types I (sialidosis type I and II), II (I-cell disease), III (pseudo-Hurler disease) and IV, mucopolysaccharide storage diseases (Hurler disease and variants, Hunter, Sanfilippo Types A,B,C,D, Morquio Types A and B, Maroteaux-Lamy and Sly diseases), Niemann-Pick disease types A/B, C1 and C2, and Schindler disease types I and II), hereditary angioedema (HAE), a copper or iron accumulation disorder (e.g., Wilson's or Menkes disease), lysosomal acid lipase deficiency, a neurological or neurodegenerative disorder, cancer, type 1 or type 2 diabetes, adenosine deaminase deficiency, a metabolic defect (e.g., glycogen storage diseases), a disease of solid organs (e.g., brain, liver, kidney, heart), or an infectious viral (e.g., hepatitis B and C, human immunodeficiency virus (HIV), etc.), bacterial or fungal disease.

Additional non-limiting examples of diseases treatable in accordance with the invention include hemostasis related disorders or bleeding disorders such as hemophilia A, hemophilia A with inhibitory antibodies, hemophilia B, hemophilia B with inhibitory antibodies, a deficiency in any coagulation Factor: VII, VIII, IX, X, XI, V, XII, II, von Willebrand factor, combined FV/FVIII deficiency, thalassemia, vitamin K epoxide reductase C1 deficiency, or gamma-carboxylase deficiency; bleeding associated with trauma, injury, thrombosis, thrombocytopenia, stroke, coagulopathy, or disseminated intravascular coagulation (DIC); over-anticoagulation associated with heparin, low molecular weight heparin, pentasaccharide, warfarin, or small molecule antithrombotics (i.e., FXa inhibitors); and platelet disorders such as, Bernard Soulier syndrome, Glanzmann thrombasthenia, and storage pool deficiency.

Non-limiting examples of heterologous nucleic acids encoding gene products (e.g., therapeutic proteins) useful in accordance with the invention include, but are not limited to GAA (acid alpha-glucosidase) for treatment of Pompe disease; TPP1 (tripeptidyl peptidase-1) for treatment of late infantile neuronal ceroid lipofuscinosis type 2 (CLN2), ATP7B (copper transporting ATPase2) for treatment of Wilson's disease; alpha galactosidase for treatment of Fabry disease; ASS1 (arginosuccinate synthase) for treatment of citrullinemia type 1; beta-glucocerebrosidase for treatment of Gaucher disease type 1; beta-hexosaminidase A for treatment of Tay Sachs disease; SERPING1 (C1 protease inhibitor; C1 esterase inhibitor) for treatment of hereditary angioedema (HAE); glucose-6-phosphatase for treatment of glycogen storage disease type I (GSDI); erythropoietin (EPO) for treatment of anemia; interferon-alpha, interferon-beta, and interferon-gamma for treatment of various immune disorders, viral infections and cancer; an interleukin (IL), including any one of IL-1 through IL-36, and corresponding receptors, for treatment of various inflammatory diseases or immuno-deficiencies; a chemokine, including chemokine (C—X—C motif) ligand 5 (CXCL5) for treatment of immune disorders; granulocyte-colony stimulating factor (G-CSF) for treatment of immune disorders such as Crohn's disease; granulocyte-macrophage colony stimulating factor (GM-CSF) for treatment of various human inflammatory diseases; macrophage colony stimulating factor (M-CSF) for treatment of various human inflammatory diseases; keratinocyte growth factor (KGF) for treatment of epithelial tissue damage; chemokines such as monocyte chemoattractant protein-1 (MCP-1) for treatment of recurrent miscarriage, HIV-related complications, and insulin resistance; tumor necrosis factor (TNF) and receptors for treatment of various immune disorders; alphal-antitrypsin for treatment of emphysema or chronic obstructive pulmonary disease (COPD); alpha-L-iduronidase for treatment of mucopolysaccharidosis I (MPS I); ornithine transcarbamoylase (OTC) for treatment of OTC deficiency; phenylalanine hydroxylase (PAH) or phenylalanine ammonia-lyase (PAL) for treatment of phenylketonuria (PKU); lipoprotein lipase for treatment of lipoprotein lipase deficiency; apolipoproteins for treatment of apolipoprotein (Apo) A-I deficiency; low-density lipoprotein receptor (LDL-R) for treatment of familial hypercholesterolemia (FH); albumin for treatment of hypoalbuminemia; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase for treatment of homocystinuria; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein; T-protein; cystic fibrosis transmembrane regulator (CFTR); and dystrophin.

In further embodiments, the heterologous polynucleotide encodes an antibody, β-globin, α-globin, spectrin, a metal transporter (ATP7A or ATP7), sulfamidase, arylsulfatase A (cerebroside-sulfatase; ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor-3 and -4, brain-derived neurotrophic factor, glial derived growth factor, transforming growth factor α, transforming growth factor β, a cytokine, α-interferon, β-interferon, interferon-γ, interleukin-2, interleukin-4, interleukin-12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1, glucokinase, guanylate cyclase 2D (LCA-GUCY2D), retinal pigment epithelium-specific 65 kDa protein (RPE65), Rab escort protein 1 (choroideremia), LCA 5 (LCA-lebercilin), ornithine ketoacid aminotransferase (gyrate atrophy), retinoschisin 1 (X-linked retinoschisis), USH1C (Usher's syndrome 1C), X-linked retinitis pigmentosa GTPase, MER proto-oncogene tyrosine kinase (MERTK), ABCA4, DFNB1 (connexin 26 deafness), ACHM 2, 3 and 4 (achromatopsia), PKD-1 or PKD-2 (polycystic kidney disease), a sulfatase, N-acetylglucosamine-1-phosphate transferase, cathepsin A, GM2-AP, NPC1, VPC2, a sphingolipid activator protein, one or more zinc finger nuclease for genome editing, and one or more donor sequence used as repair templates for genome editing.

In certain embodiments, the protein encoded by the heterologous polynucleotide comprises a gene editing nuclease. In certain aspects, the gene editing nuclease comprises a zinc finger nuclease (ZFN) or a transcription activator-like effector nuclease (TALEN). In certain aspects, the gene editing nuclease comprises a functional Type II CRISPR-Cas9.

In certain embodiments, the heterologous polynucleotide encodes an inhibitory nucleic acid. In certain aspects, the inhibitory nucleic acid is selected from the group consisting of a siRNA, an antisense molecule, miRNA, RNAi, a ribozyme and a shRNA. In certain aspects, the inhibitory nucleic acid binds to a gene, a transcript of a gene, or a transcript of a gene associated with a polynucleotide repeat disease including, but not limited to, a huntingtin (HTT) gene, a gene associated with dentatorubropallidoluysian atrophy (atrophin 1, ATN1), androgen receptor on the X chromosome in spinobulbar muscular atrophy, human Ataxin-1, -2, -3, and -7, Caᵥ2.1 P/Q voltage-dependent calcium channel (CACNA1A), TATA-binding protein, Ataxin 8 opposite strand (ATXN8OS), Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; myotonin-protein kinase (MT-PK) in myotonic dystrophy; frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercholesterolemia; HIV Tat, human immunodeficiency virus transactivator of transcription gene, in HIV infection; HIV TAR, HIV TAR, human immunodeficiency virus transactivator response element gene, in HIV infection; C—C chemokine receptor 5 (CCRS) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Reddl also known as DAN damage-inducible transcript 4 protein, in diabetic macular edema (DME) or age-related macular degeneration; vascular endothelial growth factor receptor I (VEGFR1) in age-related macular degeneration or choroidal neovascularization, caspase 2 in non-arteritic ischaemic optic neuropathy; keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and human leukocyte antigen (HLA)-allele specific in transplant; and mutant rhodopsin gene (RHO) in autosomal dominantly inherited retinitis pigmentosa (adRP).

rAAV vectors may be administered alone, or in combination with or more compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents (e.g., immunosuppressive agents) and drugs. Such biologics (proteins), agents, drugs, treatments and therapies can be administered or performed prior to, substantially contemporaneously with or following any other method or use of the invention, for example, a therapeutic method of treating a subject for a blood clotting disease such as hemophilia A or a lysosomal storage disease such as Pompe disease.

According to the invention, rAAV vectors or a combination of therapeutic agents may be administered to a subject or patient alone or in a pharmaceutically acceptable or biologically compatible composition.

The compound, agent, drug, treatment or other therapeutic regimen or protocol can be administered as a combination composition, or administered separately, such as concurrently or in series or sequentially (prior to or following) delivery or administration of a nucleic acid, vector, recombinant vector (e.g., rAAV), or recombinant virus particle of the invention. The invention therefore provides combinations in which a method or use of the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a nucleic acid, vector, recombinant vector (e.g., rAAV), or recombinant virus particle of the invention, to a subject.

As set forth herein, rAAV are useful as gene therapy vectors as they can penetrate cells and introduce nucleic acid/genetic material into the cells. Because AAV are not associated with pathogenic disease in humans, rAAV vectors are able to deliver heterologous polynucleotide sequences (e.g., therapeutic proteins and agents) to human patients without causing substantial AAV pathogenesis or disease.

rAAV vectors possess a number of desirable features for such applications, including tropism for dividing and non-dividing cells. Early clinical experience with these vectors also demonstrated no sustained toxicity and immune responses were minimal or undetectable. AAV are known to infect a wide variety of cell types in vivo and in vitro by receptor-mediated endocytosis or by transcytosis. These vector systems have been tested in humans targeting many tissues, such as, retinal epithelium, liver, skeletal muscle, airways, brain, joints and hematopoietic stem cells.

It may be desirable to introduce a rAAV vector that can provide, for example, multiple copies of a desired gene and hence greater amounts of the product of that gene. Improved rAAV vectors and methods for producing these vectors have been described in detail in a number of references, patents, and patent applications, including: Wright J. F. (Hum Gene Ther 20:698-706, 2009).

Direct delivery of rAAV vectors or ex vivo transduction of human cells followed by infusion into the body will result in expression of the heterologous nucleic acid thereby exerting a beneficial therapeutic effect on hemostasis. In the context of blood coagulation factor, such as Factor VIII, administration enhances pro-coagulation activity. In the context of an enzyme, such as GAA, administration reduces the amount or accumulation of glycogen, prevents accumulation of glycogen or degrades glycogen. This, in turn, can reduce or decrease one or more adverse effects of Pompe disease such as promoting or improving muscle tone and/or muscle strength and/or reducing or decreasing enlarged liver.

Recombinant AAV vector, as well as methods and uses thereof, include any viral strain or serotype. As a non-limiting example, a recombinant AAV vector can be based upon any AAV genome, such as Spk100, Spk200, AAV-1, -2, -3, -4, -5, -6, -7, -8, -9, -10, -11, -12, -rh74, -rh10 or AAV3B, for example. Such vectors can be based on the same strain or serotype (or subgroup or variant), or be different from each other. As a non-limiting example, a recombinant AAV vector based upon a particular serotype genome can be identical to the serotype of the capsid proteins that package the vector. In addition, a recombinant AAV vector genome can be based upon an AAV serotype genome distinct from the serotype of the AAV capsid proteins that package the vector. For example, the AAV vector genome can be based upon AAV2, whereas at least one of the three capsid proteins could be a Spk100, Spk200, AAV1, AAV3, AAV4, AAV5, AAV6, AAV7, AAV8, AAV9, AAV10, AAV11, AAV12, Rh10, Rh74 or AAV3B as well as variants thereof as disclosed herein, for example. Such AAV capsid variants include the variants of AAV capsids set forth in WO2012/145601, WO2013/158879, WO2015/013313, WO2018/156654, US2013/0059732, U.S. Pat. Nos. 9,169,299, 7,749,492, and 9,587,282.

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

As set forth herein, modified AAV capsid proteins and nucleic acids encoding the capsid proteins exhibit less than 100% sequence identity to a reference or parental AAV serotype such as Spk100, Spk200, AAV1, AAV2, AAV3, AAV4, AAV5, AAV6, AAV7, AAV8, AAV9, AAV10, AAV11, AAV12, Rh10, Rh74 or AAV3B, but are distinct from and not identical to known AAV genes or proteins, such as AAV1, AAV2, AAV3, AAV4, AAV5, AAV6, AAV7, AAV8, AAV9, AAV10, AAV11, AAV12, Rh10, Rh74 or AAV3B. In one embodiment, a modified/variant AAV capsid protein includes or consists of a sequence at least 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 99.9% identical to a reference or parental AAV capsid protein, such as Spk100, Spk200, AAV1, AAV2, AAV3, AAV4, AAV5, AAV6, AAV7, AAV8, AAV9, AAV10, AAV11, AAV12, Rh10, Rh74 or AAV3B, as well as variants of Spk100, Spk200, AAV1, AAV2, AAV3, AAV4, AAV5, AAV6, AAV7, AAV8, AAV9, AAV10, AAV11, AAV12, Rh10, Rh74 and AAV3B.

In certain embodiments, a modified/variant AAV capsid protein has 1, 2, 3, 4, 5, 5-10, 10-15, 15-20 or more amino acid substitutions. In certain embodiments, a modified/variant AAV capsid protein has a peptide insertion length of 2, 3, 4, 5, 5-10, 10-15, 15-20, 20-25, 25-30, 30-35, 35-40, 40-50 or 50-60 amino acids.

rAAV vectors may be administered to a patient via infusion in a biologically compatible carrier, for example, via intravenous injection. The rAAV vectors may optionally be encapsulated into liposomes or mixed with other phospholipids or micelles to increase stability of the molecule.

In accordance with the invention, rAAV vectors may be administered alone or in combination with other. Accordingly, rAAV vectors and other compositions, agents, drugs, biologics (proteins) can be incorporated into pharmaceutical compositions. Such pharmaceutical compositions are useful for, among other things, administration and delivery to a subject in vivo or ex vivo.

In particular embodiments, pharmaceutical compositions also contain a pharmaceutically acceptable carrier or excipient. Such excipients include any pharmaceutical agent that does not itself induce an immune response harmful to the individual receiving the composition, and which may be administered without undue toxicity.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a nucleic acid, vector, viral particle or protein to a subject.

Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, a preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran.

Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling could include amount, frequency, and method of administration.

Pharmaceutical compositions and delivery systems appropriate for the compositions, methods and uses of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

An “effective amount” or “sufficient amount” refers to an amount that provides, in single or multiple doses, alone or in combination, with one or more other compositions (therapeutic or immunosuppresive agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes/per kilogram of body weight (vg/kg), will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (protein), and the stability of the protein expressed. One skilled in the art can determine a rAAV/vector genome dose range to treat a patient having a particular disease or disorder based on the aforementioned factors, as well as other factors.

Generally, doses will range from at least 1×10⁸ vector genomes per kilogram (vg/kg) of the weight of the subject, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect. An rAAV dose in the range of 1×10¹⁰-1×10¹¹ vg/kg in mice, and 1×10¹²-1×10¹³ vg/kg in dogs have been effective. Doses can be less, for example, a dose of less than 6×10¹² vg/kg. More particularly, a dose of 5×10¹¹ vg/kg or 1×10¹² vg/kg.

rAAV vector doses can be at a level, typically at the lower end of the dose spectrum, such that there is not a substantial immune response against the heterologous nucleic acid sequence, the encoded protein or inhibitory nucleic acid, or rAAV vector. More particularly, a dose of up to but less than 6×10¹² vg/kg, such as about 5×10¹¹ to about 5×10¹² vg/kg, or more particularly, about 5×10¹¹ vg/kg or about 1×10¹² vg/kg.

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol, such as administration of recombinant enzyme (e.g., GAA) for treatment of an enzyme deficiency (e.g., Pompe disease) or administration of recombinant clotting factor protein for treatment of a clotting disorder, e.g., FVIII for treatment of hemophilia A with or without inhibitors, or FIX for treatment of hemophilia B with or without inhibitors.

Accordingly, methods and uses of the invention also include, among other things, methods and uses that result in a reduced need or use of another compound, agent, drug, therapeutic regimen, treatment protocol, process, or remedy. For example, for protein/enzyme deficiency (e.g., GAA), or a clotting factor (e.g., Factor VIII or Factor IX), a method or use of the invention has a therapeutic benefit if, in a given subject, a less frequent or reduced dose or elimination of administration of a recombinant protein/enzyme (e.g., GAA) or clotting factor (e.g., Factor VIII or Factor IX) to supplement for the deficient or defective protein in the subject. Thus, in accordance with the invention, methods and uses of reducing need or use of another treatment or therapy are provided.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease.

For Pompe disease, an effective amount would be an amount of GAA that inhibits or reduces glycogen production or accumulation, enhances or increases glycogen degradation or removal, or improves muscle tone and/or muscle strength in a subject, for example. For hemophilia A, an effective amount would be an amount that reduces frequency or severity of acute bleeding episodes in a subject, for example, or an amount that reduces clotting time as measured by a clotting assay, for example.

Therapeutic doses will depend on, among other factors, the age and general condition of the subject, the severity of the disease or disorder. A therapeutically effective amount in humans will fall in a relatively broad range that may be determined by a medical practitioner based on the response of an individual patient.

Compositions such as pharmaceutical compositions may be delivered to a subject, so as to allow production of the encoded protein or inhibitory nucleic acid. In a particular embodiment, pharmaceutical compositions comprise sufficient genetic material to enable a recipient to produce a therapeutically effective amount of a protein or inhibitory nucleic acid in the subject.

The compositions may be administered alone. In certain embodiments, a recombinant AAV particle provides a therapeutic effect without an immunosuppressive agent. The therapeutic effect optionally is sustained for a period of time, e.g., 2-4, 4-6, 6-8, 8-10, 10-14, 14-20, 20-25, 25-30, or 30-50 days or more, for example, 50-75, 75-100, 100-150, 150-200 days or more without administering an immunosuppressive agent. Accordingly, a therapeutic effect is provided for a period of time.

The compositions may be administered in combination with at least one other agent. In certain embodiments, rAAV vector is administered in conjunction with one or more immunosuppressive agents prior to, substantially at the same time or after administering a rAAV vector. In certain embodiments, e.g., 1-12, 12-24 or 24-48 hours, or 2-4, 4-6, 6-8, 8-10, 10-14, 14-20, 20-25, 25-30, 30-50, or more than 50 days following administering rAAV vector. Such administration of immunosuppressive agents after a period of time following administering rAAV vector if there is a decrease in the encoded protein or inhibitory nucleic acid after the initial expression levels for a period of time, e.g., 20-25, 25-30, 30-50, 50-75, 75-100, 100-150, 150-200 or more than 200 days following rAAV vector.

In certain embodiments, an immunosuppressive agent is an anti-inflammatory agent. In certain embodiments, an immunosuppressive agent is a steroid. In certain embodiments, an immunosuppressive agent is cyclosporine (e.g., cyclosporine A), mycophenolate, Rituximab or a derivative thereof. Additional particular agents include a stabilizing compound.

Compositions may be formulated and/or administered in any sterile, biocompatible pharmaceutical carrier, including, but not limited to, saline, buffered saline, dextrose, and water. The compositions may be formulated and/or administered to a patient alone, or in combination with other agents (e.g., co-factors). For example, in the case of a blood clotting disorder such as hemophilia A with or without inhibitors or hemophilia B with or without inhibitors, the compositions may be formulated with other agents that influence hemostasis.

Methods and uses of the invention include delivery and administration systemically, regionally or locally, or by any route, for example, by injection or infusion. Delivery of the pharmaceutical compositions in vivo may generally be accomplished via injection using a conventional syringe, although other delivery methods such as convection-enhanced delivery are envisioned (See e.g., U.S. Pat. No. 5,720,720). For example, compositions may be delivered subcutaneously, epidermally, intradermally, intrathecally, intraorbitally, intramucosally, intraperitoneally, intravenously, intra-pleurally, intraarterially, orally, intranasally, intrahepatically, via the portal vein, or intramuscularly. Other modes of administration include oral and pulmonary administration, suppositories, and transdermal applications. A clinician specializing in the treatment of patients with, for example, blood coagulation disorders may determine the optimal route for administration of the adeno-associated viral vectors based on a number of criteria, including, but not limited to: the condition of the patient and the purpose of the treatment (e.g., enhanced or reduced blood coagulation).

Invention rAAV vectors, methods and uses can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents (e.g., immunosuppressive agents) and drugs. Such biologics (proteins), agents, drugs, treatments and therapies can be administered or performed prior to, substantially contemporaneously with or following any other method or use of the invention.

The compound, agent, drug, treatment or other therapeutic regimen or protocol can be administered as a combination composition, or administered separately, such as concurrently or in series or sequentially (prior to or following) delivery or administration of a nucleic acid, vector, or rAAV particle. The invention therefore provides combinations in which a method or use of the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a nucleic acid, vector or rAAV particle of the invention, to a subject.

The invention is useful in animals including human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of protein/enzyme deficiencies such as Pompe disease, blood clotting diseases such as HemA and others known to those of skill in the art.

Subjects appropriate for treatment in accordance with the invention include those having or at risk of producing an insufficient amount or having a deficiency in a functional gene product (e.g., GAA or FVIII protein), or produce an aberrant, partially functional or non-functional gene product (e.g., GAA or FVIII protein), which can lead to disease. Subjects appropriate for treatment in accordance with the invention also include those having or at risk of producing an aberrant, or defective (mutant) gene product (protein) that leads to a disease such that reducing amounts, expression or function of the aberrant, or defective (mutant) gene product (protein) would lead to treatment of the disease, or reduce one or more symptoms or ameliorate the disease.

Subjects can be tested for an immune response, e.g., antibodies against AAV. Candidate subjects can therefore be screened prior to treatment according to a method of the invention. Subjects also can be tested for antibodies against AAV after treatment, and optionally monitored for a period of time after treatment. Subjects developing AAV antibodies can be treated with an immunosuppressive agent, or can be administered one or more additional amounts of AAV vector.

Subjects appropriate for treatment in accordance with the invention also include those having or at risk of producing antibodies against AAV. rAAV vectors can be administered or delivered to such subjects using several techniques. For example, AAV empty capsid (i.e., AAV lacking a heterologous nucleic acid) can be delivered to bind to the AAV antibodies in the subject thereby allowing the rAAV vector comprising the heterologous nucleic acid to transduce cells of the subject.

Ratio of AAV empty capsids to the rAAV vector can be between about 2:1 to about 50:1, or between about 2:1 to about 25:1, or between about 2:1 to about 20:1, or between about 2:1 to about 15:1, or between about 2:1 to about 10:1. Ratios can also be about 2:1, 3:1, 4:1, 5:1, 6:1, 7:1, 8:1, 9:1, or 10:1.

Amounts of AAV empty capsids to administer can be calibrated based upon the amount (titer) of AAV antibodies produced in a particular subject. AAV empty capsids can be of any serotype, for example, Spk100, Spk200, AAV1, AAV2, AAV3, AAV4, AAV5, AAV6, AAV7, AAV8, AAV9, AAV10, AAV11, AAV12, Rh10, Rh74, AAV3B or variants thereof as described herein.

Alternatively, or in addition to, rAAV vector can be delivered by direct intramuscular injection (e.g., one or more slow-twitch fibers of a muscle). In another alternative, a catheter introduced into the femoral artery can be used to delivery rAAV vectors to liver via the hepatic artery. Non-surgical means can also be employed, such as endoscopic retrograde cholangiopancreatography (ERCP), to deliver rAAV vectors directly to the liver, thereby bypassing the bloodstream and AAV antibodies. Other ductal systems, such as the ducts of the submandibular gland, can also be used as portals for delivering rAAV vectors into a subject that develops or has preexisting anti-AAV antibodies.

Administration or in vivo delivery to a subject can be performed prior to development of an adverse symptom, condition, complication, etc. caused by or associated with the disease. For example, a screen (e.g., genetic) can be used to identify such subjects as candidates for invention compositions, methods and uses. Such subjects therefore include those screened positive for an insufficient amount or a deficiency in a functional gene product (e.g., GAA or FVIII protein), or that produce an aberrant, partially functional or non-functional gene product (e.g., GAA or FVIII protein).

Administration or in vivo delivery to a subject in accordance with the methods and uses of the invention as disclosed herein can be practiced within 1-2, 2-4, 4-12, 12-24 or 24-72 hours after a subject has been identified as having the disease targeted for treatment, has one or more symptoms of the disease, or has been screened and is identified as positive as set forth herein even though the subject does not have one or more symptoms of the disease. Of course, methods and uses of the invention can be practiced 1-7, 7-14, 14-24, 24-48, 48-64 or more days, months or years after a subject has been identified as having the disease targeted for treatment, has one or more symptoms of the disease, or has been screened and is identified as positive as set forth herein.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. rAAV particles, and pharmaceutical compositions thereof can be packaged in single or multiple unit dosage form for ease of administration and uniformity of dosage.

Subjects can be tested for protein activity to determine if such subjects are appropriate for treatment according to a method of the invention. Subjects also can be tested for amounts of protein according to a method of the invention. Such treated subjects can be monitored after treatment periodically, e.g., every 1-4 weeks, 1-6 months, 6-12 months, or 1, 2, 3, 4, 5 or more years.

Subjects can be tested for one or more liver enzymes for an adverse response or to determine if such subjects are appropriate for treatment according to a method of the invention. Candidate subjects can therefore be screened for amounts of one or more liver enzymes prior to treatment according to a method of the invention. Subjects also can be tested for amounts of one or more liver enzymes after treatment according to a method of the invention. Such treated subjects can be monitored after treatment for elevated liver enzymes, periodically, e.g., every 1-4 weeks, 1-6 months, 6-12 months, or 1, 2, 3, 4, 5 or more years.

Exemplary liver enzymes include alanine aminotransferase (ALT), aspartate aminotransferase (AST), and lactate dehydrogenase (LDH), but other enzymes indicative of liver damage can also be monitored. A normal level of these enzymes in the circulation is typically defined as a range that has an upper level, above which the enzyme level is considered elevated, and therefore indicative of liver damage. A normal range depends in part on the standards used by the clinical laboratory conducting the assay.

The invention provides kits with packaging material and one or more components therein. A kit typically includes a label or packaging insert including a description of the components or instructions for use in vitro, in vivo, or ex vivo, of the components therein. A kit can contain a collection of such components, e.g., a rAAV particle and optionally a second active, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All patents, patent applications, publications, and other references, GenBank citations and ATCC citations cited herein are incorporated by reference in their entirety. In case of conflict, the specification, including definitions, will control.

Various terms relating to the biological molecules of the invention are used hereinabove and also throughout the specification and claims.

All of the features disclosed herein may be combined in any combination. Each feature disclosed in the specification may be replaced by an alternative feature serving a same, equivalent, or similar purpose. Thus, unless expressly stated otherwise, disclosed features (e.g., modified/variant AAV capsid proteins, rAAV particles comprising modified/variant AAV capsid proteins) are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94%, etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-850, includes ranges of 1-20, 1-30, 1-40, 1-50, 1-60, 10-30, 10-40, 10-50, 10-60, 10-70, 10-80, 20-40, 20-50, 20-60, 20-70, 20-80, 20-90, 50-75, 50-100, 50-150, 50-200, 50-250, 100-200, 100-250, 100-300, 100-350, 100-400, 100-500, 150-250, 150-300, 150-350, 150-400, 150-450, 150-500, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### Example 1

### Materials and Methods

### Cloning of the Plasmids:

The Gibson Assembly® method was used to produce the rep/cap plasmids having the modified capsid sequences with substitutions or insertions. DNA fragments used for the Gibson Assembly using NEBuilder® HiFi DNA Assembly Cloning Kit (New England Biolabs, Cat #E5520) were produced either via PCR of the parental plasmids, or ordered as gBlocks Gene Fragments from Integrated DNA Technologies. A Rep plasmid having cap sequences removed by restriction digestion was used as a backbone. The manufacturer's protocol was followed to produce the rep/cap plasmids. Several colonies from the LB plate were selected and grown overnight. The plasmids were isolated using plasmid mini prep kit (Qiagen, QIAprep® Spin Miniprep Kit, Cat #27104) and sequenced to verify the presence of the expected inserts or mutations. The selected clones with the expected inserts or mutations were further sequenced to ensure the sequence of the rest of the rep and cap genes. The selected clones were used for production of rAAV particles in small scale.

For the plasmids having transgene constructs, Renilla luciferase or Factor IX (FIX) were used as the transgene, with a Renilla luciferase gene under the control of the CAG promoter, and the FIX gene under the control of the human alpha 1-antitrypsin (hAAT) promoter.

### Small Scale Production of AAV

HEK293 cells were seeded into 6-well plates one day before the transfection. On the day of transfection, cells at 70-90% confluency were treated with a DNA-PEI mixture. Specifically, for each well, 3 μg of total DNA (1 μg of each of the helper plasmid, transgene plasmid and rep/cap plasmid) mixed with 6 μg of polyethylenimine (PEI) was used. Upon incubating the cells with DNA-PEI mixture overnight, the cell culture medium was replaced the following day. After medium replacement, the cells were grown 2 more days and the AAV viral particles were collected. For the rAAV in the cytosol, the cells were resuspended in PBS or the cell culture medium they were growing in and the suspension was transferred to 1.5 ml tubes. Instead of a detergent solvent, the cells were lysed by freeze and thaw cycles. The samples were frozen in dry ice and rapidly thawed in 37° C. water bath. The cells were transferred to dry ice as soon as they were thawed at 37° C. to prevent any possible degradation of the capsids. Before putting the samples on dry ice, the samples were vortexed to enhance the cell degradation and release of the viral particles. At the end of the 4^{th} freeze and thaw cycle, the samples were spun down at 12,000 rpm for 10 minutes with a tabletop centrifuge set to 4° C. The supernatant that contains the rAAV was transferred to a fresh tube.

### Qpcr Analysis:

While the actual samples were kept on ice for the transduction studies, small aliquots of the samples were diluted into separate tubes to be used for qPCR analysis. 2-5 μl of cell extract samples containing rAAV were diluted in ddH₂O to achieve 50- to 200-fold dilutions. The boiled samples and standards were used in three technical replicates in the assays. The transgene-containing plasmids that were used for rAAV production were used as standards. For the construct with the FIX transgene, primers ApoE-FWD (5′-TTGTCCTGGCGTGGTTTAG-3′; SEQ ID NO:127) and ApoE-REV (5′-TGCAGAATCCTTAGTGGCTG-3′; SEQ ID NO:128) were used with the probe (5′-/56-FAM/TCCACTGGT/ZEN/AGCAAGATCCCCG/3IABkFQ/-3′; SEQ ID NO:129). For the construct with the Renilla luciferase transgene, primers Renluc-FWD (5′-AGGCCGCGTTACCATGTAAA-3′; SEQ ID NO:130) and Renluc-REV (5′-TGATAACTGGTCCGCAGTGG-3′; SEQ ID NO:131) were used with the probe (5′/56-FAM/TGGGCCAGA/ZEN/TGTAAACAAATGA/3IABkFQ/-3′; SEQ ID NO:132). The qPCR analysis was done at the same day of the transduction. This assured that the actual samples that were kept on ice were not subject to another set of freeze and thaw, which might alter the quality of the intact rAAVs in the samples.

### Transduction of Cells:

Upon determining the AAV vector titers, equal amounts of vectors were used for transducing cells. The samples that were kept on ice were added to the Huh7 cells that were seeded into 24-well plates one day before the experiment. Multiplicities of infection (MOIs) ranging from 2K to 50K were used for transduction assays. Each sample was tested in duplicate or triplicate biological replicates. The media of the cells were replaced to fresh the next day. After 48-72 hours following transduction of cells, the samples were analyzed for transgene expression. For samples tested for FIX, ELISA was done directly using the undiluted media from the cells to calculate the amount of FIX secreted from the Huh7 cells, and converted to percent of control (transduction by Spark200). For the samples with Renilla luciferase transgene, the cells were lysed in passive lysis buffer provided with the Renilla luciferase assay kit. Each biological sample was split into 4 wells in the 96-well assay plate. The luciferase activities (amount of luminescence) in the lysates were calculated using a luminometer; in certain experiments, the amount of luminescence was converted to percent of the relevant control (Spark200 or Spark100).

### Example 2

Peptide Insertions into Spark200 (SEQ ID NO:1)

Several AAV LKO3 capsid (also referred to as AAV-Spark200, Spark200 and Spk200) clones with different point mutations and peptide insertions (including lipid modifying or NLS like peptides), were generated, as shown in Table 1. The peptides were inserted between amino acid positions 32-33, 34-35, 36-37, 138-139 or 139-140 on Spark200.

TABLE 1A
Spark200 and Lysine (K) → Arginine
(R) Mutant Capsids (SEQ ID NOs: 1-10)

| Spark200 | | Sequence |
|---|---|---|
| Number | Spark200 Name | Number |
| 1 | Spark200 | SEQ ID NO: 1 |
| 2 | Spark200-K137R | SEQ ID NO: 2 |
| 3 | Spark200-K528R | SEQ ID NO: 3 |
| 4 | Spark200-K533R | SEQ ID NO: 4 |
| 5 | Spark200-K545R | SEQ ID NO: 5 |
| 6 | Spark200-K528R + K533R | SEQ ID NO: 6 |
| 7 | Spark200-K137R + K528R | SEQ ID NO: 7 |
| 8 | Spark200-K137R + K533R | SEQ ID NO: 8 |
| 9 | Spark200-K137R + K545R | SEQ ID NO: 9 |
| 10 | Spark200-K137R + K528R + K533R | SEQ ID NO: 10 |

TABLE 1B
Spark200 with Peptide Insertions (SEQ ID NOs: 11-26, 42-46)

| Spark200 | | Sequence |
|---|---|---|
| Number | Spark200 Name | Number |
| 11 | Spark200_35_cmycNLS | SEQ ID NO: 11 |
| 12 | Spark200_35_PAARRVRLD | SEQ ID NO: 12 |
| 13 | Spark200_35_M9short_LR-alt | SEQ ID NO: 13 |
| 14 | Spark200_33_M9short_LR-alt | SEQ ID NO: 14 |
| 15 | Spark200_37_M9short_LR-alt | SEQ ID NO: 15 |
| 34 | Spark200_35_M9short_LK-alt1 | SEQ ID NO: 16 |
| 35 | Spark200_35_M9short_LK-alt2 | SEQ ID NO: 17 |
| 36 | Spark200_139_cmycNLS | SEQ ID NO: 18 |
| 37 | Spark200_140_cmycNLS | SEQ ID NO: 19 |
| 38 | Spark200_35_and_140_cmycNLS | SEQ ID NO: 20 |
| 39 | Spark200_140_PAARRVRLD | SEQ ID NO: 21 |
| 40 | Spark200_35_and 140_PAARRVRLD | SEQ ID NO: 22 |
| 41 | Spark200_140_M9short_LK-alt2 | SEQ ID NO: 23 |
| 42 | Spark200_35_cmycNLS_140_M9short_LK-alt2 | SEQ ID NO: 24 |
| 43 | Spark200_35_M9short_LK-alt2-and 140-cmycNLS | SEQ ID NO: 25 |
| 59 | Spark200-140hnRNP_D_NLS | SEQ ID NO: 26 |
| 60 | Spark200-140hnRNP_M_NLS | SEQ ID NO: 42 |
| 61 | Spark200-B1B_140_ptHRP_NLS | SEQ ID NO: 43 |
| 62 | Spark200-B1B_140_class2_NLS | SEQ ID NO: 44 |
| 63 | Spark200-B1B_140_class3_NLS | SEQ ID NO: 45 |
| 64* | Spark200-B1_K137R-140_cmycNLS | SEQ ID NO: 46 |

*Spark200 Number 64 (SEQ ID NO: 46) has a point mutation and a peptide insertion

For AAV capsid peptide insertions, the amino acid residue number used refers to the first residue of the insertion. Thus, for example, “139” means that the peptide is inserted immediately after amino acid residue 138 of the original capsid protein and, “140” means that the peptide is inserted immediately after amino acid residue 139 of the original capsid protein.

TABLE 1C
Spark200 Capsid Serine and Threonine
Mutants (SEQ ID NOs: 27-32)

| Spark200 | | Sequence |
|---|---|---|
| Number | Spark200 Name | Number |
| 44 | Spark200-T492V | SEQ ID NO: 27 |
| 45 | Spark200-S663V | SEQ ID NO: 28 |
| 46 | Spark200-T492V-S663V | SEQ ID NO: 29 |
| 47 | Spark200-K137R-T492V | SEQ ID NO: 30 |
| 48 | Spark200-K137R-S663V | SEQ ID NO: 31 |
| 49 | Spark200-K137R-T492V-S663V | SEQ ID NO: 32 |

TABLE 1D
Spark200 Capsids with Mutated (L/P)PXY
and/or RXXL Motifs (SEQ ID NOs: 33-41)

| Spark200 | | Sequence |
|---|---|---|
| Number | Spark200 Name | Number |
| 50 | Spark200-Y50A | SEQ ID NO: 33 |
| 51 | Spark200-Y50F | SEQ ID NO: 34 |
| 52 | Spark200-Y50E | SEQ ID NO: 35 |
| 53 | Spark200-Y252A | SEQ ID NO: 36 |
| 54 | Spark200-Y252F | SEQ ID NO: 37 |
| 55 | Spark200-L171A | SEQ ID NO: 38 |
| 56 | Spark200-L479A | SEQ ID NO: 39 |
| 57 | Spark200-L517A | SEQ ID NO: 40 |
| 58 | Spark200-N691A | SEQ ID NO: 41 |

TABLE 1E
Spark200 Capsids with Cell Penetrating
Peptide Inserts (SEQ ID NOs: 47-55)

| | | | | Peptide |
|---|---|---|---|---|
| | | Spark200 | | Insertion |
| Spark 200 | | Sequence | | Sequence |
| Number | Spark200 Name | Number | Sequence of Peptide Insertion | Number |
| 65 | Spark200- | SEQ ID | GLFDIIKKIAESF | SEQ ID |
| | 35_GLFDIIKKIAESF | NO: 47 | | NO: 133 |
| | (aurein 1.2) | | | |
| 66 | Spark200- | SEQ ID | FFLIPKG | SEQ ID |
| | 35_FFLIPKG | NO: 48 | | NO: 134 |
| 67 | Spark200- | SEQ ID | GWTLNSAGYLLGKINLKALAALAKKIL | SEQ ID |
| | 35_transportan | NO: 49 | | NO: 135 |
| 68 | Spark200- | SEQ ID | GRKKRRQRRRPPQ | SEQ ID |
| | 35_TAT-H | NO: 50 | | NO: 136 |
| 69 | Spark200- | SEQ ID | KKAAAVLLPVLLAAP | SEQ ID |
| | 35_KKAAAVLLPVLLAAP | NO: 51 | | NO: 137 |
| 70 | Spark200- | SEQ ID | RQIKIWFQNRRLKWKKGG | SEQ ID |
| | 35_penetratin_MLmut | NO: 52 | | NO: 138 |
| 71 | Spark200- | SEQ ID | RRRRRRRRRRRRRRRR | SEQ ID |
| | 35_R16 | NO: 53 | | NO: 139 |
| 72 | Spark200- | SEQ ID | KKKKKKKKKKKKKKKK | SEQ ID |
| | 35_K16 | NO: 54 | | NO: 140 |
| 73 | Spark200- | SEQ ID | RRRRRRRRRRRRRRRRLRVRLASHLRKLR | SEQ ID |
| | 35_R16_ApoE | NO: 55 | KRLLRDA | NO: 141 |

The AAV vectors were produced using a small-scale production system in HEK293 cells in 6-well dishes, as described in the materials and methods section (Example 1). The titers of the AAV vectors that are produced in this small scale were calculated using qPCR. Upon calculating vector titer, vectors were used at equal MOIs to transduce cells growing in 24-well plates. For most of the studies, Huh7 cells were used. However, HepG2 and HEK293 cells were also used to evaluate the transduction properties of novel capsids in these cell types.

Renilla luciferase was the transgene used in most of the AAV vector productions. For the studies using the AAV vectors with this transgene, luciferase activity in the transduced cells was measured using a luminometer. AAV vectors were used at varying MOIs, from 2K to 5K, for luciferase activity. For some of the studies, Factor IX (FIX) was used as a transgene and the FIX levels secreted into the media were determined by ELISA. The MOIs used for the FIX-containing AAV vectors ranged from 5K to 50K. Only the results of the cells in the same plate were used as a comparison, to minimize the effect of plate to plate variability. Each AAV vector was evaluated in duplicate or triplicate biological copies. For studies with Renilla luciferase, each of the biological replicates was transferred to 96-well plate as a quadruplicate technical repeat after lysing with the lysis buffer. For ELISA of FIX containing samples, each biological replicate was analyzed in duplicate technical repeat in the plate. These technical and biological repeats decreased internal variability of the results.

More than 50 Spark200 variants were evaluated, including peptide insertions and point mutations. Most of the peptide insertions either decreased production of AAV vector or did not substantially increase the AAV vector transduction effectively in vitro. The presence of methionine in the peptide sequence typically decreases AAV vector production and, consistent with this, methionine strongly decreased AAV vector production. As some of the peptides contain methionine in their sequences, the methionine residues were modified to leucine (e.g., M9 (variants shown in Table 2) and penetratin peptide (Table 1E)) or alanine (class3 NLS, Kosugi et al., 2009, J. Biol. Chem., 284:478-485). The amino acid sequences of representative nonlimiting inserted nuclear localization peptides are shown in Table 2.

TABLE 2
Peptides Inserted into Spark200 (LK03) Capsid (SEQ ID NOs: 84-92)

| Peptide Name | Peptide Sequence | Sequence Number |
|---|---|---|
| M9 like NLS-1 | FGNYNNQSSNFGPLRGGNFGGRSSGPY | SEQ ID NO: 84 |
| M9 like NLS-2 | FGNYNNQSSNFGPLKGGNFGGRSSGPY | SEQ ID NO: 85 |
| cmycNLS | PAAKRVKLD | SEQ ID NO: 86 |
| cmyc like NLS | PAARRVRLD | SEQ ID NO: 87 |
| hnRNP_D_NLS | YGDYSNQQSGYGKVSRRGGHQNSYKPY | SEQ ID NO: 88 |
| hnRNP_M_NLS | GEGERPAQNEKRKEKNIKRGGNRFEPY | SEQ ID NO: 89 |
| ptHRP_NLS | RYLTQETNKVETYKEQPLKTPGKKKKGKP | SEQ ID NO: 90 |
| class2_NLS | RAAKRPRTT | SEQ ID NO: 91 |
| class3_NLS | AAAKRSWSAAF | SEQ ID NO: 92 |

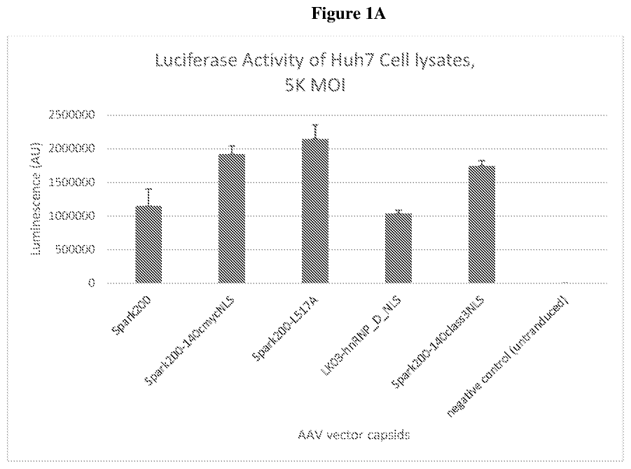

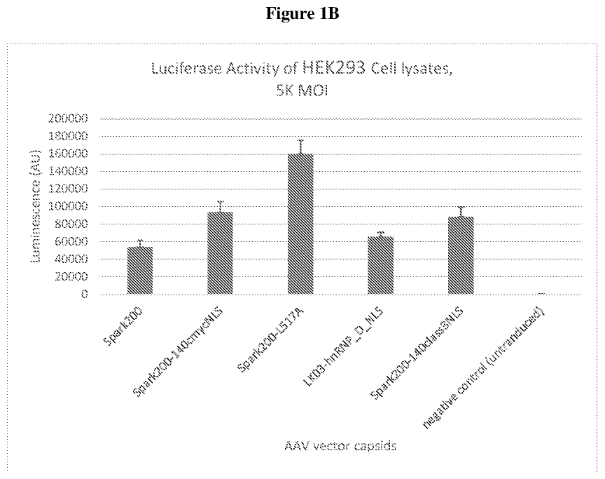

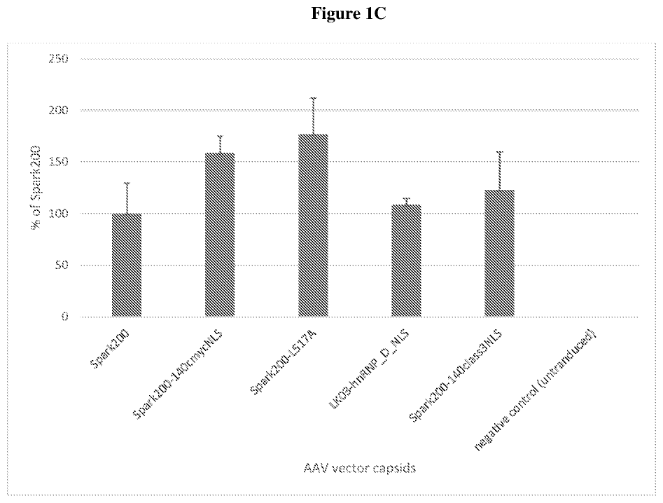

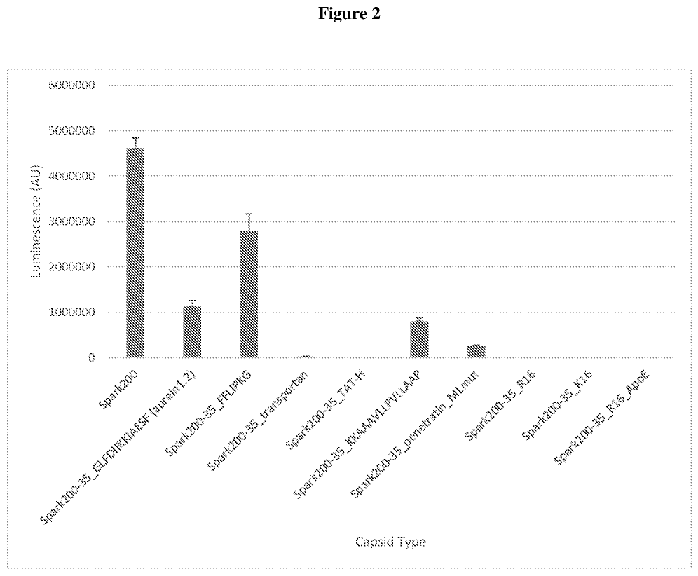

Point mutations changing possible ubiquitin ligase binding sites ((L/P)PXY and RXXL motifs) mostly resulted in a decrease of AAV vector production. The list of Spark200 variants with these modifications is shown in Table 1D. Among those that did not affect AAV vector production, only the L517A (Spark200-L517A; SEQ ID NO:40) mutant showed significant increase in cell transduction; yielding 2- to 3-fold higher transduction as compared to wild type Spark200 (FIGS. 1A-C).

The studies with peptide insertions showed that the most effective AAV vectors in terms of cell transduction had 2 different insertions at the beginning of VP2 region (FIGS. 1A-C). These insertions were a classical cmycNLS (PAAKRVKLD, SEQ ID NO:86) and a modified version of a class 3 NLS (AAAKRSWSAAF, SEQ ID NO:92) inserted between amino acid position 139 and amino acid position 140. This region is at the beginning of VP2 and is just before the start of the BR2 region of AAV capsid, which acts as an essential NLS for viral transduction.

These insertions increased the AAV vector transduction by two-fold in different studies using Renilla luciferase constructs in Huh7 cells (FIG. 1A). These findings were repeated in the HEK293 cell line, as well, using Renilla luciferase (FIG. 1B).

Good performing AAV vectors using a FIX transgene that transduced Huh7 cells were also produced. The amount of FIX levels secreted to the media was higher than the wild type Spark200, confirming better transduction of these AAV vectors (FIG. 1C).

Additionally, results with the Spark200 construct having the hnRNP_D_NLS insertion are shown in FIG. 1C. This insertion is different from the others in terms of direct binding to karyopherin beta without a need for an importin alpha. While cmycNLS and class3-NLS cannot bind to importin beta and need another carrier, namely importin alpha, to facilitate the binding, hnRNP_D-NLS can bind to importin beta directly. Although hnRNP_D-NLS did not detectably increase Spark200 transduction, the production yield and transduction rate were similar to those of wild type Spark200, unlike most of the similar insertions, (M9_NLS, ptHRP_NLS, hnRNP_MNLS) that can bind to importin beta directly. The presence of certain transport receptors/importins in other cell types may result in these constructs being effective in the context of in vivo transduction.

Most of the lysine, threonine and serine point mutations did not change AAV vector production, but did not detectably increase AAV vector transduction significantly in vitro.

Peptide Insertions into Spark100 (SEO ID NO:59)
Insertions after BR1 and Before BR2
The effects of NLS peptide insertions into Spark100 (also referred to as Spk100) were also studied. Single (1×) or multiple tandem repeats of the peptides (2× or 3×) were inserted into regions of the Spark100 capsid sequence such that VP1 and VP2 capsid proteins, but not VP3, contain the insertions (Table 3 lists the NLS peptide-modified Spark100 clones, as well as several Spark100 clones with point mutations). Table 4 presents the sequences of Spark100 peptide inserts.

TABLE 3
Spark100 Clones

| | Spark100 | Sequence |
|---|---|---|
| Spark100 Name | Number | Number |
| Spark100 | Spark100_X01 | SEQ ID NO: 59 |
| Spark100_K137R | Spark100_X02 | SEQ ID NO: 60 |
| Spk100_L519A | Spark100_X03 | SEQ ID NO: 61 |
| Spk100-140-cmycNLS | Spark100_X04 | SEQ ID NO: 62 |
| Spk100-140-hnRNP_D_NLS | Spark100_X05 | SEQ ID NO: 63 |
| Spk100-140-class3NLS | Spark100_X06 | SEQ ID NO: 64 |
| Spk100-140-SV40 NLS | Spark100_X07 | SEQ ID NO: 65 |
| Spk100-140-2XcmycNLS | Spark100_X08 | SEQ ID NO: 66 |
| Spk100-140-2xclass3NLS | Spark100_X09 | SEQ ID NO: 67 |
| Spk100-140-2X-SV40 NLS | Spark100_X10 | SEQ ID NO: 68 |
| Spk100-140-1xclass3NLS- | Spark100_X11 | SEQ ID NO: 69 |
| 1X cmycNLS | | |
| Spk100-140-1XcmycNLS- | Spark100_X12 | SEQ ID NO: 70 |
| 1Xclass3NLS | | |
| Spk100-140-3XSV40 NLS | Spark100_X13 | SEQ ID NO: 71 |
| Spk100-140-3XcmycNLS | Spark100_X14 | SEQ ID NO: 72 |
| Spk100-140-3xclass3NLS | Spark100_X15 | SEQ ID NO: 73 |
| Spk100-140-2XcmycNLS- | Spark100_X16 | SEQ ID NO: 74 |
| 1Xclass3NLS | | |
| Spk100-140-2xclass3NLS- | Spark100_X17 | SEQ ID NO: 75 |
| 1X cmycNLS | | |
| Spk100-140-1X hnRNP_D_NLS - | Spark100_X18 | SEQ ID NO: 76 |
| 1Xclass3NLS-1X cmycNLS | | |
| Spk100-L519A-140-cmycNLS | Spark100_X19 | SEQ ID NO: 77 |
| Spk100-163-cmycNLS | Spark100_X20 | SEQ ID NO: 78 |
| Spk100-163-2XcmycNLS | Spark100_X21 | SEQ ID NO: 79 |
| Spk100-163-SV40 NLS | Spark100_X22 | SEQ ID NO: 80 |
| Spk100-163-2X-SV40 NLS | Spark100_X23 | SEQ ID NO: 81 |
| Spk100-163-class3NLS | Spark100_X24 | SEQ ID NO: 82 |
| Spk100-163-2xclass3NLS | Spark100_X25 | SEQ ID NO: 83 |

TABLE 4
Peptides Inserted into Spark100 Capsid
(SEQ ID NOs: 86, 93-97, 92, 98, 99, 88 and 100-104)

| | | Sequence |
|---|---|---|
| Peptide Name | Peptide Sequence | Number |
| cmycNLS | PAAKRVKLD | SEQ ID NO: 86 |
| 2X cmycNLS | PAAKRVKLDEDPAAKRVKLD | SEQ ID NO: 93 |
| 3X cmycNLS | PAAKRVKLDEDPAAKRVKLDEDPAAKRVKLD | SEQ ID NO: 94 |
| SV40 NLS | PKKKRKV | SEQ ID NO: 95 |
| 2X-SV40 NLS | PKKKRKVEDPKKKRKV | SEQ ID NO: 96 |
| 3X-SV40 NLS | PKKKRKVEDPKKKRKVEDPKKKRKV | SEQ ID NO: 97 |
| Class3 NLS | AAAKRSWSAAF | SEQ ID NO: 92 |
| 2X Class3 NLS | AAAKRSWSAAFEDAAAKRSWSAAF | SEQ ID NO: 98 |
| 3X Class3 NLS | AAAKRSWSAAFEDAAAKASWSAAFEDAAAKRSWSAAF | SEQ ID NO: 99 |
| hnRNP-D-NLS | YGDYSNQQSGYGKVSRRGGHQNSYKPY | SEQ ID NO: 88 |
| 1xclass3_NLS-1XcmycNLS | AAAKRSWSAAFEDPAAKRVKLD | SEQ ID NO: 100 |
| 1XcmycNLS-1x class3_NLS | PAAKRVKLDEDAAAKRSWSAAF | SEQ ID NO: 101 |
| 2XcmycNLS-1Xclass3_NLS | PAAKRVKLDEDPAAKRVKLDEDAAAKRSWSAAF | SEQ ID NO: 102 |
| 2xclass3_NLS-1XcmyxNLS | AAAKRSWSAAFEDAAAKRSWSAAFEDPAAKRVKLD | SEQ ID NO: 103 |
| 1X_hnRNP_D_NLS- | YGDYSNQQSGYGKVSRRGGHQNSYKPYEDAAAKRSWS | SEQ ID NO: 104 |
| 1Xclass3NLS-1X_cmycNLS | AAFEDPAAKRVKLD | |

AAV vectors with the variant Spark100 capsids were produced in small scale in 6-well plates, as described in Example 1. Renilla luciferase was the transgene for these AAV vectors. The AAV vectors were evaluated for transduction of Huh7 cells, and luciferase activity in the cells was determined 48 or 72 hours after AAV vector transduction. In addition to the cmycNLS and class3 NLS that increased the Spark200 transduction, SV40 NLS was also evaluated for Spark100.

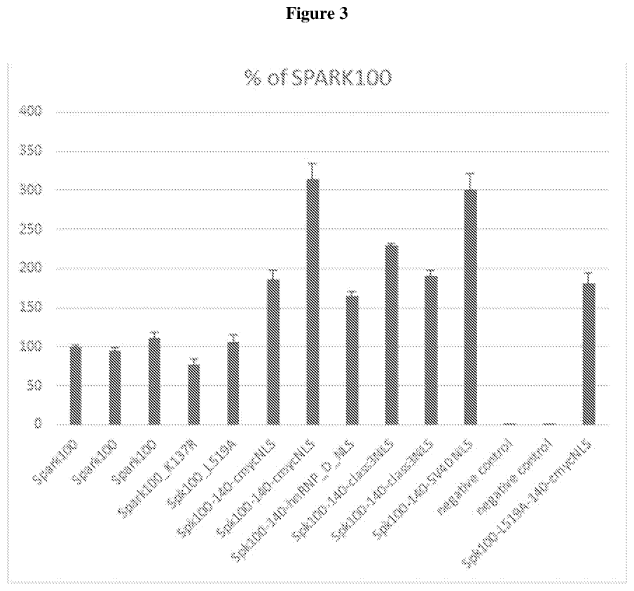

In vitro studies showed up to a 3-fold increase in the cell transduction by 1×NLS insert-containing Spark100 clones (FIG. 3). The AAV vector production yields for these AAV vectors were slightly less than for the wild type Spark100, but at comparable levels (Table 5).

The studies with modified Spark200 showed that one of the most effective modifications in terms of transduction was the Spark200-L517A mutant, having a mutated RXXL motif. In contrast to the Spark200 results, the analogous mutation in Spark100 (L519A; RXXL mutant) did not measurably increase transduction compared to wild type Spark100 particles (FIG. 3).

TABLE 5
Yields of Spark100 with amino acid point mutations and
1X NLS insertions in small scale AAV vector production

| Spark100 name | % of Wild Type |
|---|---|
| Spark100 | 100 |
| Spark100_K137R | 85-109 |
| Spk100_L517A | 30-36 |
| Spk100-140-cmycNLS | 56-70 |
| Spk100-140-hnRNP_D_NLS | 77-94 |
| Spk100-140-class3NLS | 47-77 |
| Spk100-140-SV40 NLS | 30-47 |
| Spk100-L519A-140-cmycNLS | 54-66 |

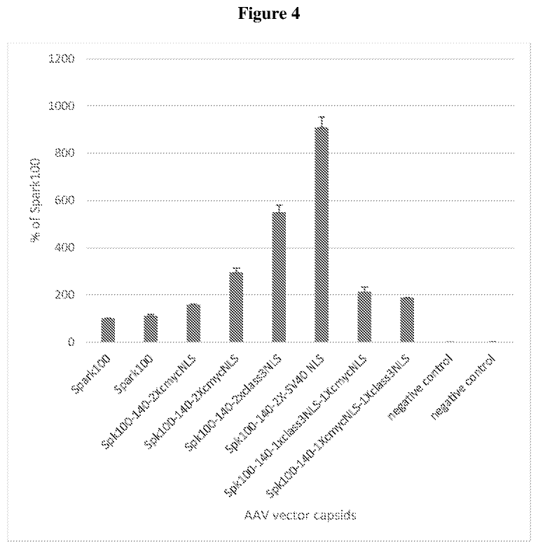

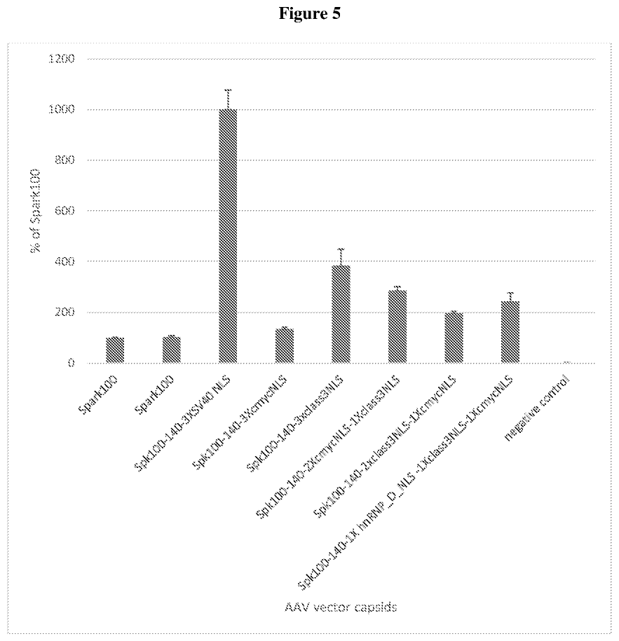

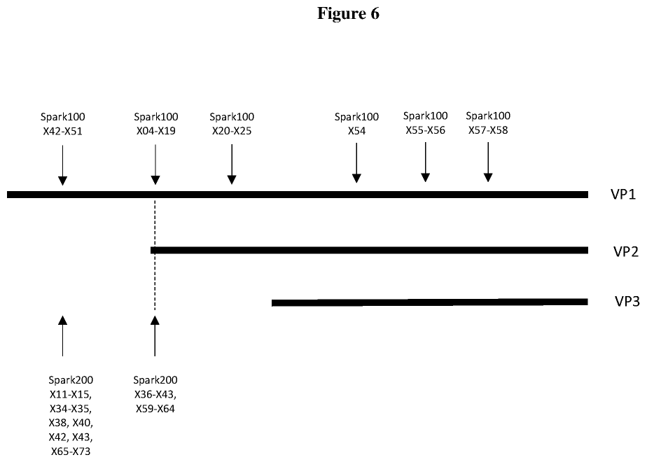

Tandem 2× and 3× insertions of peptides into the Spark100 VP2 region, before BR2, were also evaluated; the peptides are shown in Table 4. AAV vector yield using the above-described small-scale production methods with the Renilla luciferase transgene was reduced (Tables 6 and 7). The largest reduction in AAV vector yield for 2× tandem repeats was observed for Spk100-140-2×class3NLS (also referred to as SparkX09) and Spk100-140-2×SV40 NLS (also referred to as SparkX10). Almost all of the vectors with capsids with the 2× tandem repeat insertions of the same or different NLSs showed 2-fold or greater transduction of Huh7 cells, as compared to wild type Spark100, as determined by luciferase activity (FIGS. 4 and 5). The most effective transductions were observed for vectors with capsids with 2× or 3× class 3 NLS or 2× or 3×SV40 NLSs, which had a 70-90% decrease in AAV vector yield.

TABLE 6
Yields of Spark100 2X NLS insertions
in small scale AAV vector production

| Spark100 name | % of Wild Type |
|---|---|
| Spark100 | 100 |
| Spk100-140-2XcmycNLS | 50-67 |
| Spk100-140-2xclass3NLS | 6-10 |
| Spk100-140-2X-SV40 NLS | 13-25 |
| Spk100-140-1xclass3NLS-1XcmycNLS | 38-46 |
| Spk100-140-1XcmycNLS-1Xclass3NLS | 76-91 |

TABLE 7
Yields of Spark100 3X NLS insertions
in small scale AAV vector production

| Spark100 name | % of Wild Type |
|---|---|
| Spark100 | 100 |
| Spk100-140-3XSV40 NLS | 8-11.8 |
| Spk100-140-3XcmycNLS | 37-55 |
| Spk100-140-3xclass3NLS | 4-7.5 |
| Spk100-140-2XcmcyNLS-1Xclass3NLS | 22-25.3 |
| Spk100-140-2xclass3NLS-1XcmcyNLS | 6-8.3 |
| Spk100-140-1XhnRNP_D_NLS- | 5.8-7 |
| 1Xclass3NLS-1XcmycNLS | |

### Insertions Between BR2 and BR3

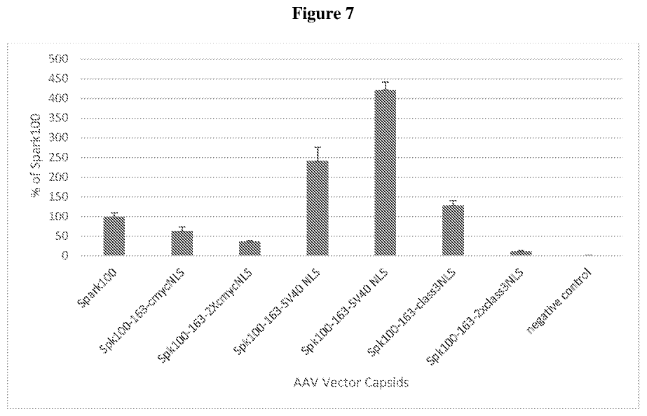

The data show that insertions into the region between BR2 and BR3 can increase transduction without significantly decreasing AAV vector yield (FIG. 7 and Table 8). Renilla luciferase was the transgene for this study.

TABLE 8
Yields of Spark100 1X and Spark100 2X NLS insertions between
BR2 and BR3 in small scale AAV vector production

| Spark100 name | % of wild type |
|---|---|
| Spark100 | 100 |
| Spk100-163-cmycNLS | 84-88 |
| Spk100-163-2XcmycNLS | 31-45 |
| Spk100-163-SV40 NLS | 73-108 |
| Spk100-163-SV40 NLS | 65-73 |
| Spk100-163-class3NLS | 67-123 |
| Spk100-163-2Xclass3NLS | 5-6 |

### Example 3

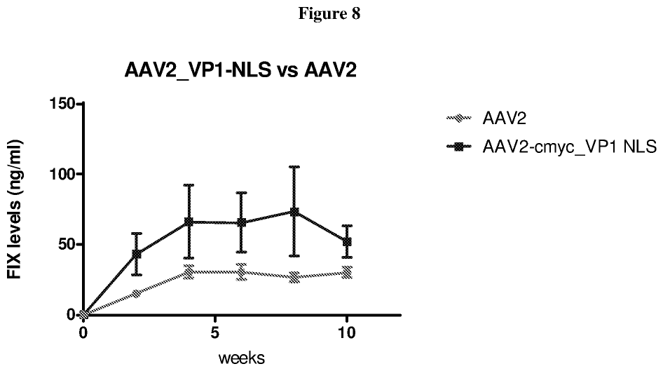

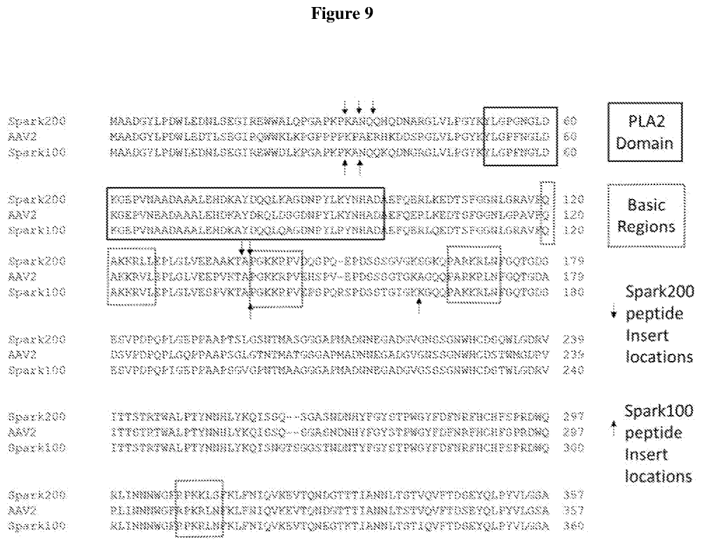

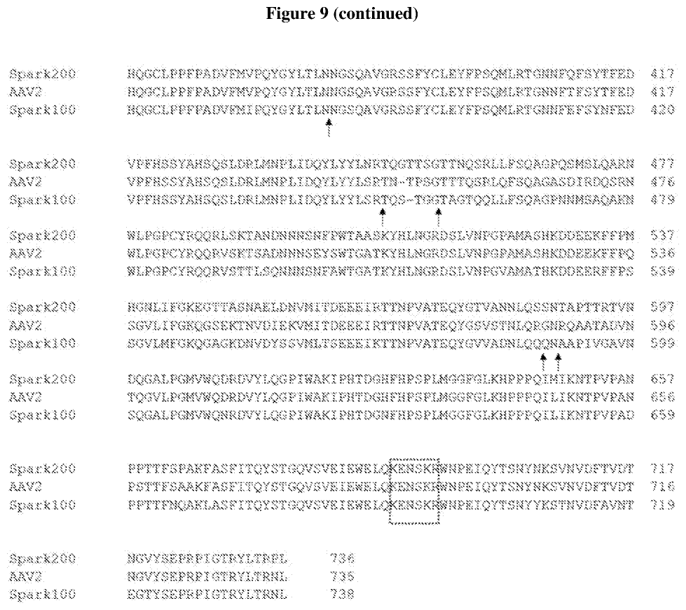

Initial in vivo (C57BL/6 mice) studies were performed with an AAV vector comprising an AAV2 capsid with a cmycNLS (PAAKRVKLD; SEQ ID NO:86) inserted between amino acid residues 34-35. The transgene was FIX and FIX levels (ng/mL of plasma) were detected by ELISA. The data show that rAAV particles with cmycNLS (PAAKRVKLD; SEQ ID NO:86) inserted between amino acid residues 34-35 of the AAV2 capsid yielded higher transduction (based on ELISA-detected levels of FIX transgene product) than did rAAV particles having the wild type AAV2 capsid without the NLS insertion (FIG. 8).

Based upon the initial data from in vitro studies, the following clones were also evaluated in vivo:

### Spark100-X07 (Spk100-140-1×SV40 NLS)

Spark100-X09 (Spk100-140-2× class3 NLS)

### Spark100-X10 (Spk100-140-2×SV40 NLS)

rAAV vectors carrying an acid alpha-glucosidase (GAA) transgene were prepared, and the yields for 5 roller bottle (small scale) production were as follows:

Spark100: 6.82×E12 vg/mL (1.7 ml)
Spark100-X07 (Spk100-140-1×SV40 NLS): 6.2×E12 vg/mL (1.45 ml)
Spark100-X09 (Spk100-140-2× class3 NLS): 6.02×E10 vg/mL (0.8 ml)
Spark100-X10(Spk100-140-2×SV40 NLS): 1.21×E13 vg/mL (1.5 ml)

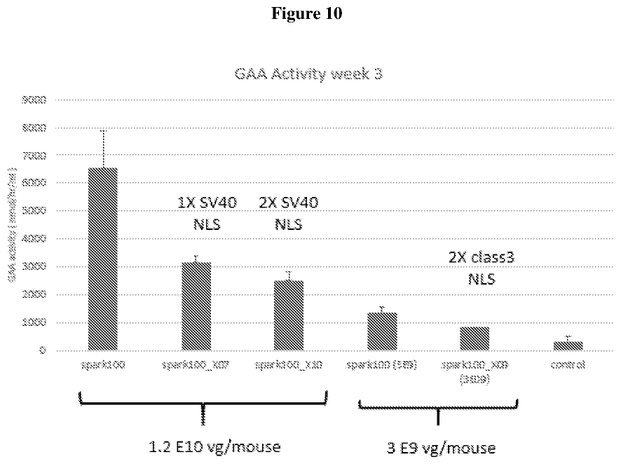

C57BL/6 mice were injected with a dose of either 1.2×E10 vg/mouse or 3×E9 vg/mouse and plasma GAA enzyme activity was determined by a standard GAA activity assay three weeks post administration. Administration of the rAAV vectors with the modified capsids resulted in measurable plasma GAA activity in vivo, with levels of enzyme activity lower than achieved with rAAV having wild type Spark100 capsid (FIG. 10).

In a further study, the following were evaluated and in vivo:

Spark100 (Spk100 wild type)

### Spark100-X04 (Spark100-140-cmycNLS)

### Spark100-X05 (Spark100-140-hnRNP D NLS)

Spark100-X06 (Spark100-140-class3 NLS)

### Spark100-X08 (Spark100-140-2×cmycNLS)

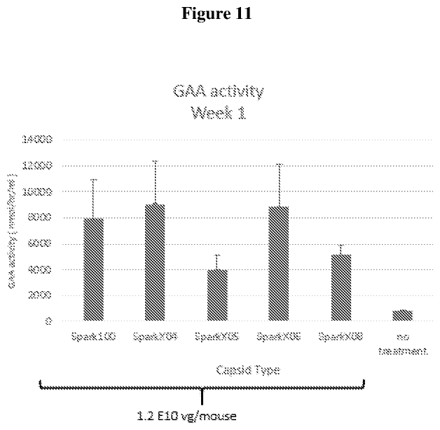

C57BL/6 mice were injected at a dose of 1.2E10 vg/animal. The modified AAV vectors could still produce GAA enzyme that was detected (based on a standard GAA activity assay) in the plasma one week post administration. The amount of GAA production was comparable to that from rAAV vectors with wild type Spark100 capsids, with Spark100-X04 (Spark100-140-cmycNLS) and Spark100-X06 (Spark100-140-class3 NLS) showing a modest increase over wild type Spark100 (FIG. 11).

### Example 4

TABLE 9
Spark100 with peptide insertions or Lysine
(K) → Arginine (R) substitutions

| | Spark100 | Sequence |
|---|---|---|
| Spark100 Name | Number | Number |
| Spark100-33-cmycNLS | Spark100-X42 | SEQ ID NO: 105 |
| Spark100-35-cmycNLS | Spark100-X43 | SEQ ID NO: 106 |
| Spark100-33-2xcmycNLS | Spark100-X44 | SEQ ID NO: 107 |
| Spark100-35-2xcmycNLS | Spark100-X45 | SEQ ID NO: 108 |
| Spark100-35-3xcmycNLS | Spark100-X46 | SEQ ID NO: 109 |
| Spark100-35-1xClass3NLS | Spark100-X47 | SEQ ID NO: 110 |
| Spark100-35-2xClass3NLS | Spark100-X48 | SEQ ID NO: 111 |
| Spark100-35-3xClass3NLS | Spark100-X49 | SEQ ID NO: 112 |
| Spark100-35-hnRNP_NLS | Spark100-X50 | SEQ ID NO: 113 |
| Spark100-35-1x-SV40 NLS | Spark100-X51 | SEQ ID NO: 114 |
| Spark100_K333R | Spark100-X52 | SEQ ID NO: 115 |
| Spark100_K530R | Spark100-X53 | SEQ ID NO: 116 |
| Spark100_385cmycNLS | Spark100-X54 | SEQ ID NO: 117 |
| Spark100_451cmycNLS | Spark100-X55 | SEQ ID NO: 118 |
| Spark100_457cmycNLS | Spark100-X56 | SEQ ID NO: 119 |
| Spark100_589cmycNLS | Spark100-X57 | SEQ ID NO: 120 |
| Spark100_591cmycNLS | Spark100-X58 | SEQ ID NO: 121 |

AAV vectors were prepared using either Spark100 or modified Spark100 capsids (see Table 9) and tested in vitro on Huh7 cells. The transgene used for AAV vector production was Renilla luciferase.

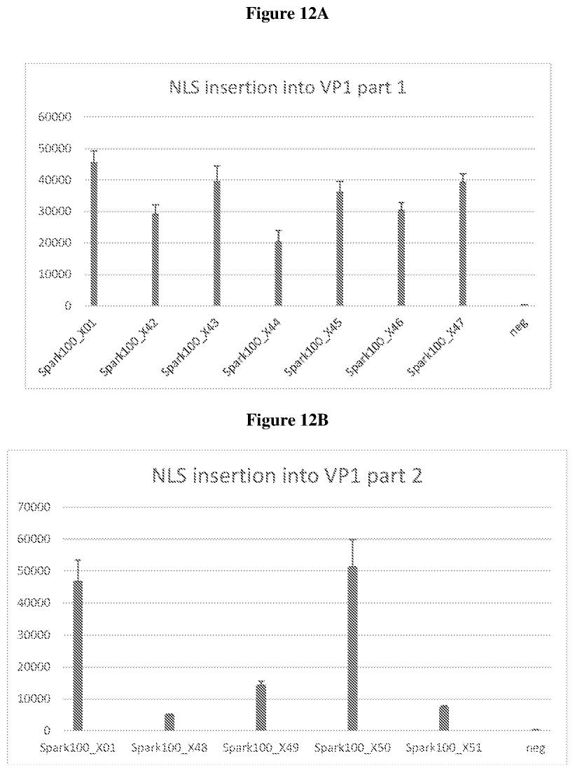

Luciferase activities of the Huh7 cells transduced with rAAV vectors having various NLS insertions in only VP1 of the Spark100 capsid, and carrying the Renilla luciferase transgene are shown in FIGS. 12A and 12B (refer also to Table 9, above). The AAV vectors were produced in small scale, and luciferase activity was determined 48 hours after transduction. The AAV vectors with these NLS insertions yielded similar or less luciferase activity compared to the AAV vectors produced with wild type Spark100 capsid in this study.

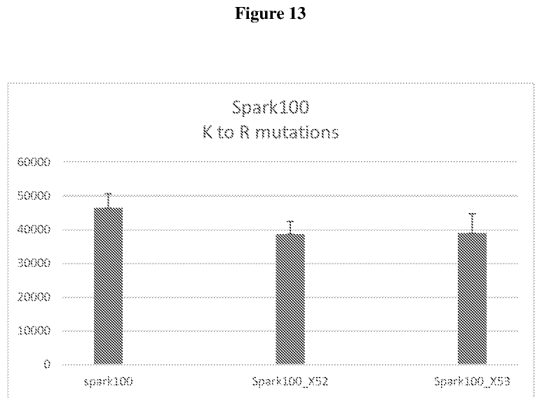

Luciferase activity of the Huh7 cells transduced with capsids Spark100-X52 and Spark100-X53 are shown in FIG. 13 (refer also to Table 9, above). Lysine (K) residues at position 333 or 530 of Spark100 capsid were mutated to arginine (R). These lysine residues are present in VP1, VP2 and VP3 of Spark100. Renilla luciferase was used as the transgene and the AAV vectors were produced in small scale. 48 hours after transduction, the luciferase activity was calculated/determined. The AAV vectors with additional K→R point mutations in the capsid yield similar luciferase activity compared to the AAV vectors produced with wild type Spark100 capsid in this study.

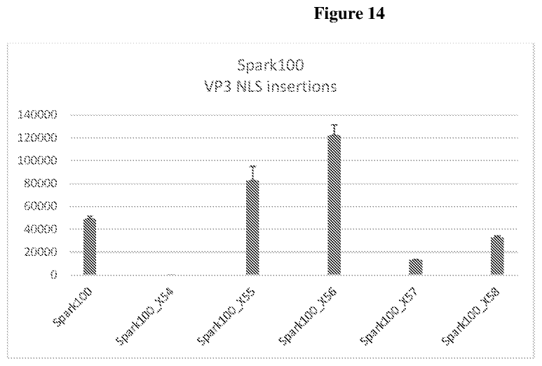

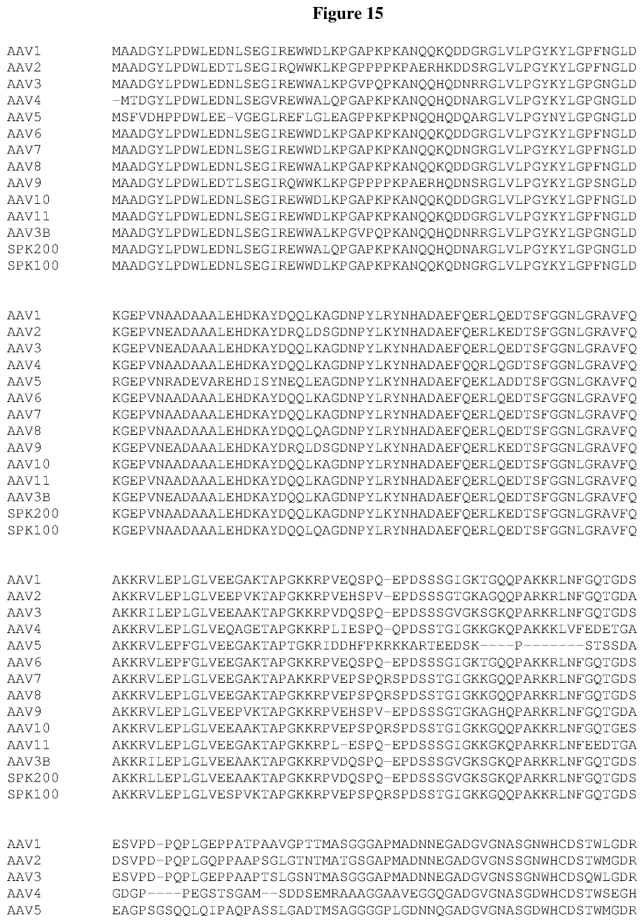

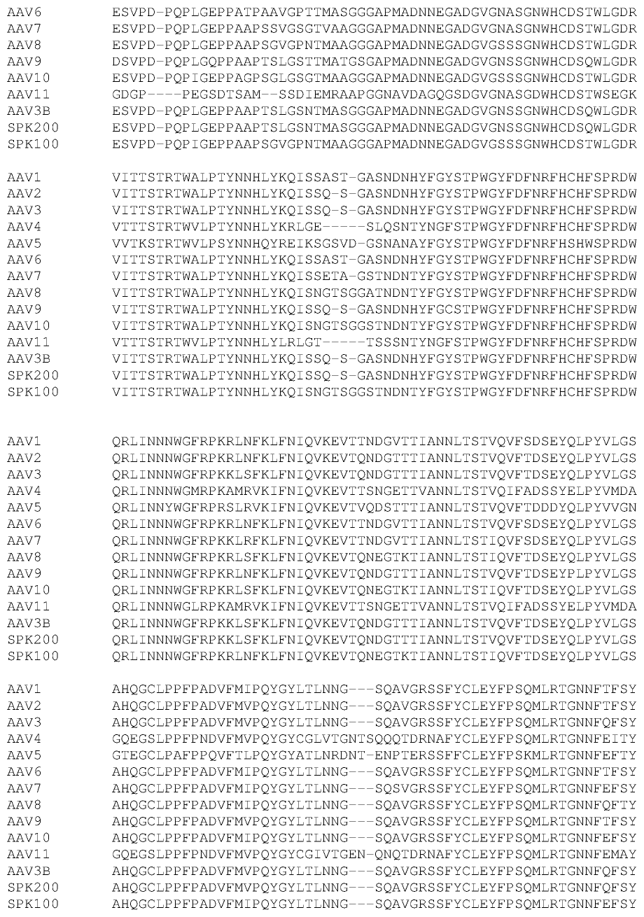

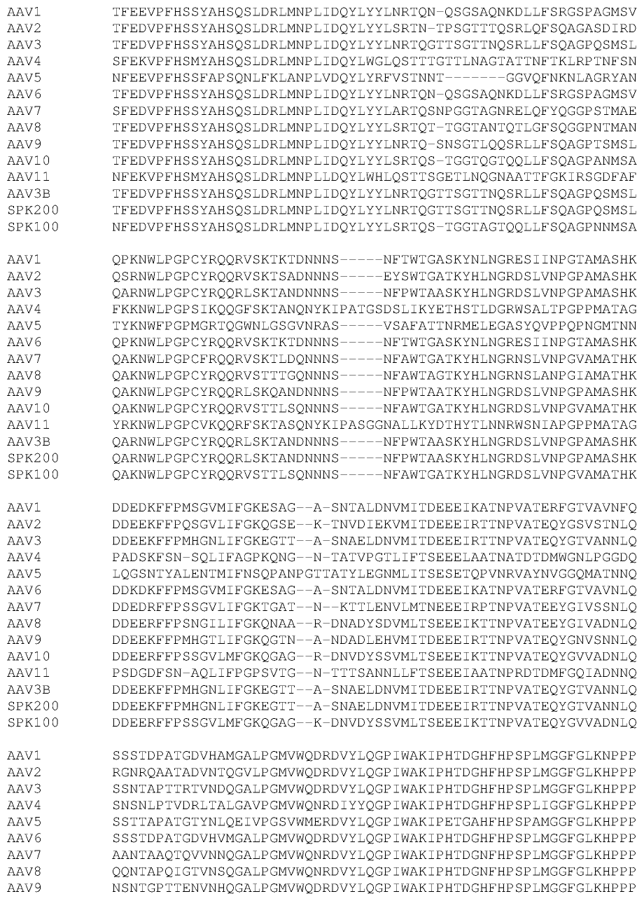

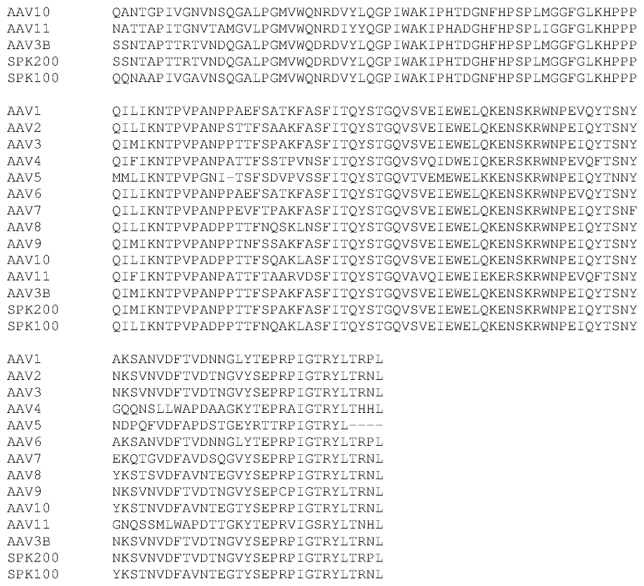

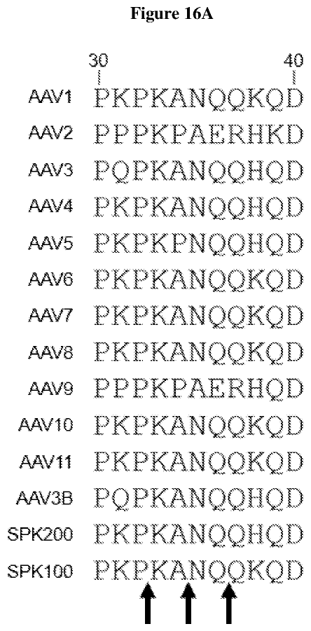

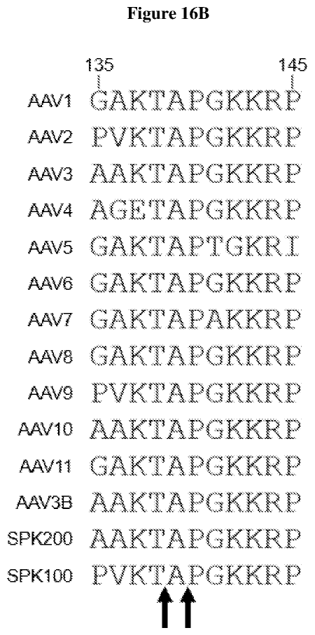

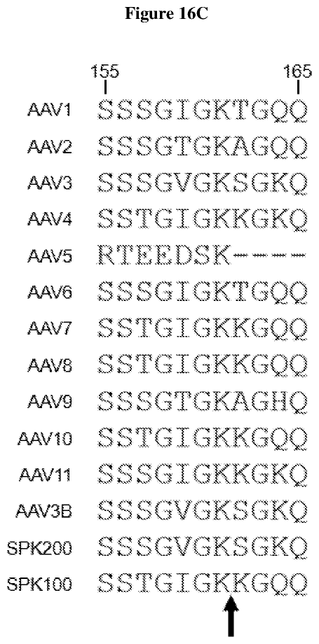

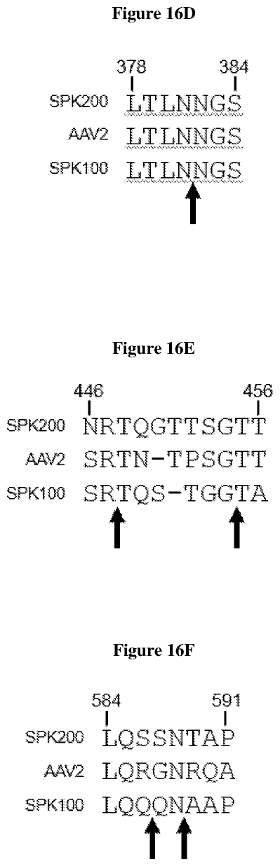

Luciferase activity of the Huh7 cells transduced with NLS inserts in the VP3 domain of Spark100 capsid are shown in FIG. 14 (refer also to Table 9, above). Renilla luciferase was used as the transgene and the AAV vectors were produced in small scale. Luciferase activity was determined 48 hours after transduction. The AAV vectors with capsids having NLS insertions at amino acid positions 450-451 (Spark100_451cmycNLS) and 456-457 (Spark100_457cmycNLS) yielded higher luciferase activity than the vectors produced with Spark100 capsid.

### Example 5

As disclosed herein, an increase in transduction upon peptide insertions into either Spark100, Spark200 or AAV2 capsids has been observed in both in vitro and in vivo studies. The NLS peptides in these studies that were most effective were cmycNLS, SV40 NLS and Class3 type NLS.

### Example 6

AMINO ACID SEQUENCES of SPARK200 (Spk200) and
SPARK200 (Spk200) VARIANTS

LK03-X01:
Wild type Spk200 (SEQ ID NO: 1)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X02:
pLK03PK-K137R (SEQ ID NO: 2)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAARTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X03:
pLK03PK-K528R (SEQ ID NO: 3)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHRDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X04:
pLK03PK-K533R (SEQ ID NO: 4)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEERFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X05:
pLK03PK-K545R (SEQ ID NO: 5)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGREGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X06:
pLK03PK-K528R-K533R (SEQ ID NO: 6)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHRDDEERFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X07:
pLK03PK-K137R-K528R (SEQ ID NO: 7)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAARTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHRDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X08:
pLK03PK-K137R-K533R (SEQ ID NO: 8)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAARTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEERFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X09:
pLK03PK-K137R-K545R (SEQ ID NO: 9)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAARTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGREGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X10:
pLK03PK-K137R-K528R-K533R (SEQ ID NO: 10)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAARTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHRDDEERFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X11:
pLK03PK-35-cmycNLS (SEQ ID NO: 11)
MAADGYLPDWLEDNLSEGIREWWALQPGAPKPKAPAAKRVKLDNQQHQDNARGLVLPGYKYLGP
GNGLDKGEPVNAADAAALEHDKAYDQQLKAGDNPYLKYNHADAEFQERLKEDTSFGGNLGRAVF
QAKKRLLEPLGLVEEAAKTAPGKKRPVDQSPQEPDSSSGVGKSGKQPARKRLNFGQTGDSESVP
DPQPLGEPPAAPTSLGSNTMASGGGAPMADNNEGADGVGNSSGNWHCDSQWLGDRVITTSTRTW
ALPTYNNHLYKQISSQSGASNDNHYFGYSTPWGYFDFNRFHCHFSPRDWQRLINNNWGFRPKKL
SFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFQFSYTFEDVPFHSSYAHSQSLDRLMNPLID
QYLYYLNRTQGTTSGTTNQSRLLFSQAGPQSMSLQARNWLPGPCYRQQRLSKTANDNNNSNFPW
TAASKYHLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHP
SPLMGGFGLKHPPPQIMIKNTPVPANPPTTFSPAKFASFITQYSTGQVSVEIEWELQKENSKRW
NPEIQYTSNYNKSVNVDFTVDTNGVYSEPRPIGTRYLTRPL
LK03-X12:
pLK03PK-35-PAARRVRLD (SEQ ID NO: 12)
MAADGYLPDWLEDNLSEGIREWWALQPGAPKPKAPAARRVRLDNQQHQDNARGLVLPGYKYLGP
GNGLDKGEPVNAADAAALEHDKAYDQQLKAGDNPYLKYNHADAEFQERLKEDTSFGGNLGRAVF
QAKKRLLEPLGLVEEAAKTAPGKKRPVDQSPQEPDSSSGVGKSGKQPARKRLNFGQTGDSESVP
DPQPLGEPPAAPTSLGSNTMASGGGAPMADNNEGADGVGNSSGNWHCDSQWLGDRVITTSTRTW
ALPTYNNHLYKQISSQSGASNDNHYFGYSTPWGYFDFNRFHCHFSPRDWQRLINNNWGFRPKKL
SFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFQFSYTFEDVPFHSSYAHSQSLDRLMNPLID
QYLYYLNRTQGTTSGTTNQSRLLFSQAGPQSMSLQARNWLPGPCYRQQRLSKTANDNNNSNFPW
TAASKYHLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHP
SPLMGGFGLKHPPPQIMIKNTPVPANPPTTFSPAKFASFITQYSTGQVSVEIEWELQKENSKRW
NPEIQYTSNYNKSVNVDFTVDTNGVYSEPRPIGTRYLTRPL
LK03-X13:
pLK03PK-35-M9-LR-alternative (SEQ ID NO: 13)
MAADGYLPDWLEDNLSEGIREWWALQPGAPKPKAFGNYNNQSSNFGPLRGGNFGGRSSGPYNQQ
HQDNARGLVLPGYKYLGPGNGLDKGEPVNAADAAALEHDKAYDQQLKAGDNPYLKYNHADAEFQ
ERLKEDTSFGGNLGRAVFQAKKRLLEPLGLVEEAAKTAPGKKRPVDQSPQEPDSSSGVGKSGKQ
PARKRLNFGQTGDSESVPDPQPLGEPPAAPTSLGSNTMASGGGAPMADNNEGADGVGNSSGNWH
CDSQWLGDRVITTSTRTWALPTYNNHLYKQISSQSGASNDNHYFGYSTPWGYFDFNRFHCHFSP
RDWQRLINNNWGFRPKKLSFKLFNIQVKEVTQNDGTTTIANNLTSTVQVFTDSEYQLPYVLGSA
HQGCLPPFPADVFMVPQYGYLTLNNGSQAVGRSSFYCLEYFPSQMLRTGNNFQFSYTFEDVPFH
SSYAHSQSLDRLMNPLIDQYLYYLNRTQGTTSGTTNQSRLLFSQAGPQSMSLQARNWLPGPCYR
QQRLSKTANDNNNSNFPWTAASKYHLNGRDSLVNPGPAMASHKDDEEKFFPMHGNLIFGKEGTT
ASNAELDNVMITDEEEIRTTNPVATEQYGTVANNLQSSNTAPTTRTVNDQGALPGMVWQDRDVY
LQGPIWAKIPHTDGHFHPSPLMGGFGLKHPPPQIMIKNTPVPANPPTTFSPAKFASFITQYSTG
QVSVEIEWELQKENSKRWNPEIQYTSNYNKSVNVDFTVDTNGVYSEPRPIGTRYLTRPL
LK03-X14:
pLK03PK-33-M9-LR-alternative (SEQ ID NO: 14)
MAADGYLPDWLEDNLSEGIREWWALQPGAPKPFGNYNNQSSNFGPLRGGNFGGRSSGPYKANQQ
HQDNARGLVLPGYKYLGPGNGLDKGEPVNAADAAALEHDKAYDQQLKAGDNPYLKYNHADAEFQ
ERLKEDTSFGGNLGRAVFQAKKRLLEPLGLVEEAAKTAPGKKRPVDQSPQEPDSSSGVGKSGKQ
PARKRLNFGQTGDSESVPDPQPLGEPPAAPTSLGSNTMASGGGAPMADNNEGADGVGNSSGNWH
CDSQWLGDRVITTSTRTWALPTYNNHLYKQISSQSGASNDNHYFGYSTPWGYFDFNRFHCHFSP
RDWQRLINNNWGFRPKKLSFKLFNIQVKEVTQNDGTTTIANNLTSTVQVFTDSEYQLPYVLGSA
HQGCLPPFPADVFMVPQYGYLTLNNGSQAVGRSSFYCLEYFPSQMLRTGNNFQFSYTFEDVPFH
SSYAHSQSLDRLMNPLIDQYLYYLNRTQGTTSGTTNQSRLLFSQAGPQSMSLQARNWLPGPCYR
QQRLSKTANDNNNSNFPWTAASKYHLNGRDSLVNPGPAMASHKDDEEKFFPMHGNLIFGKEGTT
ASNAELDNVMITDEEEIRTTNPVATEQYGTVANNLQSSNTAPTTRTVNDQGALPGMVWQDRDVY
LQGPIWAKIPHTDGHFHPSPLMGGFGLKHPPPQIMIKNTPVPANPPTTFSPAKFASFITQYSTG
QVSVEIEWELQKENSKRWNPEIQYTSNYNKSVNVDFTVDTNGVYSEPRPIGTRYLTRPL
LK03-X15:
pLK03PK-37-M9-LR-alternative (SEQ ID NO: 15)
MAADGYLPDWLEDNLSEGIREWWALQPGAPKPKANQFGNYNNQSSNFGPLRGGNFGGRSSGPYQ
HQDNARGLVLPGYKYLGPGNGLDKGEPVNAADAAALEHDKAYDQQLKAGDNPYLKYNHADAEFQ
ERLKEDTSFGGNLGRAVFQAKKRLLEPLGLVEEAAKTAPGKKRPVDQSPQEPDSSSGVGKSGKQ
PARKRLNFGQTGDSESVPDPQPLGEPPAAPTSLGSNTMASGGGAPMADNNEGADGVGNSSGNWH
CDSQWLGDRVITTSTRTWALPTYNNHLYKQISSQSGASNDNHYFGYSTPWGYFDFNRFHCHFSP
RDWQRLINNNWGFRPKKLSFKLFNIQVKEVTQNDGTTTIANNLTSTVQVFTDSEYQLPYVLGSA
HQGCLPPFPADVFMVPQYGYLTLNNGSQAVGRSSFYCLEYFPSQMLRTGNNFQFSYTFEDVPFH
SSYAHSQSLDRLMNPLIDQYLYYLNRTQGTTSGTTNQSRLLFSQAGPQSMSLQARNWLPGPCYR
QQRLSKTANDNNNSNFPWTAASKYHLNGRDSLVNPGPAMASHKDDEEKFFPMHGNLIFGKEGTT
ASNAELDNVMITDEEEIRTTNPVATEQYGTVANNLQSSNTAPTTRTVNDQGALPGMVWQDRDVY
LQGPIWAKIPHTDGHFHPSPLMGGFGLKHPPPQIMIKNTPVPANPPTTFSPAKFASFITQYSTG
QVSVEIEWELQKENSKRWNPEIQYTSNYNKSVNVDFTVDTNGVYSEPRPIGTRYLTRPL
LK03-X34:
pLK03PK-35-M9-LK-alternative 1 (SEQ ID NO: 16)
MAADGYLPDWLEDNLSEGIREWWALQPGAPKPKAFGNYNNQSSNFGPLKGGNFGGRSSGPYNQQ
HQDNARGLVLPGYKYLGPGNGLDKGEPVNAADAAALEHDKAYDQQLKAGDNPYLKYNHADAEFQ
ERLKEDTSFGGNLGRAVFQAKKRLLEPLGLVEEAAKTAPGKKRPVDQSPQEPDSSSGVGKSGKQ
PARKRLNFGQTGDSESVPDPQPLGEPPAAPTSLGSNTMASGGGAPMADNNEGADGVGNSSGNWH
CDSQWLGDRVITTSTRTWALPTYNNHLYKQISSQSGASNDNHYFGYSTPWGYFDFNRFHCHFSP
RDWQRLINNNWGFRPKKLSFKLFNIQVKEVTQNDGTTTIANNLTSTVQVFTDSEYQLPYVLGSA
HQGCLPPFPADVFMVPQYGYLTLNNGSQAVGRSSFYCLEYFPSQMLRTGNNFQFSYTFEDVPFH
SSYAHSQSLDRLMNPLIDQYLYYLNRTQGTTSGTTNQSRLLFSQAGPQSMSLQARNWLPGPCYR
QQRLSKTANDNNNSNFPWTAASKYHLNGRDSLVNPGPAMASHKDDEEKFFPMHGNLIFGKEGTT
ASNAELDNVMITDEEEIRTTNPVATEQYGTVANNLQSSNTAPTTRTVNDQGALPGMVWQDRDVY
LQGPIWAKIPHTDGHFHPSPLMGGFGLKHPPPQIMIKNTPVPANPPTTFSPAKFASFITQYSTG
QVSVEIEWELQKENSKRWNPEIQYTSNYNKSVNVDFTVDTNGVYSEPRPIGTRYLTRPL
LK03-X35:
pLK03PK-35-M9-LK-alternative 2 (SEQ ID NO: 17)
MAADGYLPDWLEDNLSEGIREWWALQPGAPKPKAFGNYNNQSSNFGPLKGGNFGGRSSGPYNQQ
HQDNARGLVLPGYKYLGPGNGLDKGEPVNAADAAALEHDKAYDQQLKAGDNPYLKYNHADAEFQ
ERLKEDTSFGGNLGRAVFQAKKRLLEPLGLVEEAAKTAPGKKRPVDQSPQEPDSSSGVGKSGKQ
PARKRLNFGQTGDSESVPDPQPLGEPPAAPTSLGSNTMASGGGAPMADNNEGADGVGNSSGNWH
CDSQWLGDRVITTSTRTWALPTYNNHLYKQISSQSGASNDNHYFGYSTPWGYFDFNRFHCHFSP
RDWQRLINNNWGFRPKKLSFKLFNIQVKEVTQNDGTTTIANNLTSTVQVFTDSEYQLPYVLGSA
HQGCLPPFPADVFMVPQYGYLTLNNGSQAVGRSSFYCLEYFPSQMLRTGNNFQFSYTFEDVPFH
SSYAHSQSLDRLMNPLIDQYLYYLNRTQGTTSGTTNQSRLLFSQAGPQSMSLQARNWLPGPCYR
QQRLSKTANDNNNSNFPWTAASKYHLNGRDSLVNPGPAMASHKDDEEKFFPMHGNLIFGKEGTT
ASNAELDNVMITDEEEIRTTNPVATEQYGTVANNLQSSNTAPTTRTVNDQGALPGMVWQDRDVY
LQGPIWAKIPHTDGHFHPSPLMGGFGLKHPPPQIMIKNTPVPANPPTTFSPAKFASFITQYSTG
QVSVEIEWELQKENSKRWNPEIQYTSNYNKSVNVDFTVDTNGVYSEPRPIGTRYLTRPL
LK03-X36:
pLK03PK-139-cmycNLS (SEQ ID NO: 18)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTPAAKRVKLDAPGKKRPVDQSPQEPDSSSGVGKSGKQPARKRLNFGQTGDSESVP
DPQPLGEPPAAPTSLGSNTMASGGGAPMADNNEGADGVGNSSGNWHCDSQWLGDRVITTSTRTW
ALPTYNNHLYKQISSQSGASNDNHYFGYSTPWGYFDFNRFHCHFSPRDWQRLINNNWGFRPKKL
SFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFQFSYTFEDVPFHSSYAHSQSLDRLMNPLID
QYLYYLNRTQGTTSGTTNQSRLLFSQAGPQSMSLQARNWLPGPCYRQQRLSKTANDNNNSNFPW
TAASKYHLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHP
SPLMGGFGLKHPPPQIMIKNTPVPANPPTTFSPAKFASFITQYSTGQVSVEIEWELQKENSKRW
NPEIQYTSNYNKSVNVDFTVDTNGVYSEPRPIGTRYLTRPL
LK03-X37:
pLK03PK-140-cmycNLS (SEQ ID NO: 19)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPAAKRVKLDPGKKRPVDQSPQEPDSSSGVGKSGKQPARKRLNFGQTGDSESVP
DPQPLGEPPAAPTSLGSNTMASGGGAPMADNNEGADGVGNSSGNWHCDSQWLGDRVITTSTRTW
ALPTYNNHLYKQISSQSGASNDNHYFGYSTPWGYFDFNRFHCHFSPRDWQRLINNNWGFRPKKL
SFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFQFSYTFEDVPFHSSYAHSQSLDRLMNPLID
QYLYYLNRTQGTTSGTTNQSRLLFSQAGPQSMSLQARNWLPGPCYRQQRLSKTANDNNNSNFPW
TAASKYHLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHP
SPLMGGFGLKHPPPQIMIKNTPVPANPPTTFSPAKFASFITQYSTGQVSVEIEWELQKENSKRW
NPEIQYTSNYNKSVNVDFTVDTNGVYSEPRPIGTRYLTRPL
LK03-X38:
pLK03PK-35 and 140-cmycNLS (SEQ ID NO: 20)
MAADGYLPDWLEDNLSEGIREWWALQPGAPKPKAPAAKRVKLDNQQHQDNARGLVLPGYKYLGP
GNGLDKGEPVNAADAAALEHDKAYDQQLKAGDNPYLKYNHADAEFQERLKEDTSFGGNLGRAVF
QAKKRLLEPLGLVEEAAKTAPAAKRVKLDPGKKRPVDQSPQEPDSSSGVGKSGKQPARKRLNFG
QTGDSESVPDPQPLGEPPAAPTSLGSNTMASGGGAPMADNNEGADGVGNSSGNWHCDSQWLGDR
VITTSTRTWALPTYNNHLYKQISSQSGASNDNHYFGYSTPWGYFDFNRFHCHFSPRDWQRLINN
NWGFRPKKLSFKLFNIQVKEVTQNDGTTTIANNLTSTVQVFTDSEYQLPYVLGSAHQGCLPPFP
ADVFMVPQYGYLTLNNGSQAVGRSSFYCLEYFPSQMLRTGNNFQFSYTFEDVPFHSSYAHSQSL
DRLMNPLIDQYLYYLNRTQGTTSGTTNQSRLLFSQAGPQSMSLQARNWLPGPCYRQQRLSKTAN
DNNNSNFPWTAASKYHLNGRDSLVNPGPAMASHKDDEEKFFPMHGNLIFGKEGTTASNAELDNV
MITDEEEIRTTNPVATEQYGTVANNLQSSNTAPTTRTVNDQGALPGMVWQDRDVYLQGPIWAKI
PHTDGHFHPSPLMGGFGLKHPPPQIMIKNTPVPANPPTTFSPAKFASFITQYSTGQVSVEIEWE
LQKENSKRWNPEIQYTSNYNKSVNVDFTVDTNGVYSEPRPIGTRYLTRPL
LK03-X39:
pLK03PK-140-PAARRVRLD(SEQ ID NO: 21)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPAARRVRLDPGKKRPVDQSPQEPDSSSGVGKSGKQPARKRLNFGQTGDSESVP
DPQPLGEPPAAPTSLGSNTMASGGGAPMADNNEGADGVGNSSGNWHCDSQWLGDRVITTSTRTW
ALPTYNNHLYKQISSQSGASNDNHYFGYSTPWGYFDFNRFHCHFSPRDWQRLINNNWGFRPKKL
SFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFQFSYTFEDVPFHSSYAHSQSLDRLMNPLID
QYLYYLNRTQGTTSGTTNQSRLLFSQAGPQSMSLQARNWLPGPCYRQQRLSKTANDNNNSNFPW
TAASKYHLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHP
SPLMGGFGLKHPPPQIMIKNTPVPANPPTTFSPAKFASFITQYSTGQVSVEIEWELQKENSKRW
NPEIQYTSNYNKSVNVDFTVDTNGVYSEPRPIGTRYLTRPL
LK03-X40:
pLK03PK-35 and 140-PAARRVRLD(SEQ ID NO :22)
MAADGYLPDWLEDNLSEGIREWWALQPGAPKPKAPAARRVRLDNQQHQDNARGLVLPGYKYLGP
GNGLDKGEPVNAADAAALEHDKAYDQQLKAGDNPYLKYNHADAEFQERLKEDTSFGGNLGRAVF
QAKKRLLEPLGLVEEAAKTAPAARRVRLDPGKKRPVDQSPQEPDSSSGVGKSGKQPARKRLNFG
QTGDSESVPDPQPLGEPPAAPTSLGSNTMASGGGAPMADNNEGADGVGNSSGNWHCDSQWLGDR
VITTSTRTWALPTYNNHLYKQISSQSGASNDNHYFGYSTPWGYFDFNRFHCHFSPRDWQRLINN
NWGFRPKKLSFKLFNIQVKEVTQNDGTTTIANNLTSTVQVFTDSEYQLPYVLGSAHQGCLPPFP
ADVFMVPQYGYLTLNNGSQAVGRSSFYCLEYFPSQMLRTGNNFQFSYTFEDVPFHSSYAHSQSL
DRLMNPLIDQYLYYLNRTQGTTSGTTNQSRLLFSQAGPQSMSLQARNWLPGPCYRQQRLSKTAN
DNNNSNFPWTAASKYHLNGRDSLVNPGPAMASHKDDEEKFFPMHGNLIFGKEGTTASNAELDNV
MITDEEEIRTTNPVATEQYGTVANNLQSSNTAPTTRTVNDQGALPGMVWQDRDVYLQGPIWAKI
PHTDGHFHPSPLMGGFGLKHPPPQIMIKNTPVPANPPTTFSPAKFASFITQYSTGQVSVEIEWE
LQKENSKRWNPEIQYTSNYNKSVNVDFTVDTNGVYSEPRPIGTRYLTRPL
LK03-X41:
pLK03PK-140_M9-LK-alternative 2 (SEQ ID NO :23)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAFGNYNNQSSNFGPLKGGNFGGRSSGPYPGKKRPVDQSPQEPDSSSGVGKSGKQ
PARKRLNFGQTGDSESVPDPQPLGEPPAAPTSLGSNTMASGGGAPMADNNEGADGVGNSSGNWH
CDSQWLGDRVITTSTRTWALPTYNNHLYKQISSQSGASNDNHYFGYSTPWGYFDFNRFHCHFSP
RDWQRLINNNWGFRPKKLSFKLFNIQVKEVTQNDGTTTIANNLTSTVQVFTDSEYQLPYVLGSA
HQGCLPPFPADVFMVPQYGYLTLNNGSQAVGRSSFYCLEYFPSQMLRTGNNFQFSYTFEDVPFH
SSYAHSQSLDRLMNPLIDQYLYYLNRTQGTTSGTTNQSRLLFSQAGPQSMSLQARNWLPGPCYR
QQRLSKTANDNNNSNFPWTAASKYHLNGRDSLVNPGPAMASHKDDEEKFFPMHGNLIFGKEGTT
ASNAELDNVMITDEEEIRTTNPVATEQYGTVANNLQSSNTAPTTRTVNDQGALPGMVWQDRDVY
LQGPIWAKIPHTDGHFHPSPLMGGFGLKHPPPQIMIKNTPVPANPPTTFSPAKFASFITQYSTG
QVSVEIEWELQKENSKRWNPEIQYTSNYNKSVNVDFTVDTNGVYSEPRPIGTRYLTRPL
LK03-X42:
pLK03PK-35-cmycNLSand140_M9-LK-alternative 2 (SEQ ID NO: 24)
MAADGYLPDWLEDNLSEGIREWWALQPGAPKPKAPAAKRVKLDNQQHQDNARGLVLPGYKYLGP
GNGLDKGEPVNAADAAALEHDKAYDQQLKAGDNPYLKYNHADAEFQERLKEDTSFGGNLGRAVF
QAKKRLLEPLGLVEEAAKTAFGNYNNQSSNFGPLKGGNFGGRSSGPYPGKKRPVDQSPQEPDSS
SGVGKSGKQPARKRLNFGQTGDSESVPDPQPLGEPPAAPTSLGSNTMASGGGAPMADNNEGADG
VGNSSGNWHCDSQWLGDRVITTSTRTWALPTYNNHLYKQISSQSGASNDNHYFGYSTPWGYFDF
NRFHCHFSPRDWQRLINNNWGFRPKKLSFKLFNIQVKEVTQNDGTTTIANNLTSTVQVFTDSEY
QLPYVLGSAHQGCLPPFPADVFMVPQYGYLTLNNGSQAVGRSSFYCLEYFPSQMLRTGNNFQFS
YTFEDVPFHSSYAHSQSLDRLMNPLIDQYLYYLNRTQGTTSGTTNQSRLLFSQAGPQSMSLQAR
NWLPGPCYRQQRLSKTANDNNNSNFPWTAASKYHLNGRDSLVNPGPAMASHKDDEEKFFPMHGN
LIFGKEGTTASNAELDNVMITDEEEIRTTNPVATEQYGTVANNLQSSNTAPTTRTVNDQGALPG
MVWQDRDVYLQGPIWAKIPHTDGHFHPSPLMGGFGLKHPPPQIMIKNTPVPANPPTTFSPAKFA
SFITQYSTGQVSVEIEWELQKENSKRWNPEIQYTSNYNKSVNVDFTVDTNGVYSEPRPIGTRYL
TRPL
LK03-X43:
pLK03PK-35-M9-LK-alt2_140-cmycNLS (SEQ ID NO: 25)
MAADGYLPDWLEDNLSEGIREWWALQPGAPKPKAFGNYNNQSSNFGPLKGGNFGGRSSGPYNQQ
HQDNARGLVLPGYKYLGPGNGLDKGEPVNAADAAALEHDKAYDQQLKAGDNPYLKYNHADAEFQ
ERLKEDTSFGGNLGRAVFQAKKRLLEPLGLVEEAAKTAPAAKRVKLDPGKKRPVDQSPQEPDSS
SGVGKSGKQPARKRLNFGQTGDSESVPDPQPLGEPPAAPTSLGSNTMASGGGAPMADNNEGADG
VGNSSGNWHCDSQWLGDRVITTSTRTWALPTYNNHLYKQISSQSGASNDNHYFGYSTPWGYFDF
NRFHCHFSPRDWQRLINNNWGFRPKKLSFKLFNIQVKEVTQNDGTTTIANNLTSTVQVFTDSEY
QLPYVLGSAHQGCLPPFPADVFMVPQYGYLTLNNGSQAVGRSSFYCLEYFPSQMLRTGNNFQFS
YTFEDVPFHSSYAHSQSLDRLMNPLIDQYLYYLNRTQGTTSGTTNQSRLLFSQAGPQSMSLQAR
NWLPGPCYRQQRLSKTANDNNNSNFPWTAASKYHLNGRDSLVNPGPAMASHKDDEEKFFPMHGN
LIFGKEGTTASNAELDNVMITDEEEIRTTNPVATEQYGTVANNLQSSNTAPTTRTVNDQGALPG
MVWQDRDVYLQGPIWAKIPHTDGHFHPSPLMGGFGLKHPPPQIMIKNTPVPANPPTTFSPAKFA
SFITQYSTGQVSVEIEWELQKENSKRWNPEIQYTSNYNKSVNVDFTVDTNGVYSEPRPIGTRYL
TRPL
LK03-X59:
LK03-14OhnRNP_D_NLS (SEQ ID NO: 26)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAYGDYSNQQSGYGKVSRRGGHQNSYKPYPGKKRPVDQSPQEPDSSSGVGKSGKQ
PARKRLNFGQTGDSESVPDPQPLGEPPAAPTSLGSNTMASGGGAPMADNNEGADGVGNSSGNWH
CDSQWLGDRVITTSTRTWALPTYNNHLYKQISSQSGASNDNHYFGYSTPWGYFDFNRFHCHFSP
RDWQRLINNNWGFRPKKLSFKLFNIQVKEVTQNDGTTTIANNLTSTVQVFTDSEYQLPYVLGSA
HQGCLPPFPADVFMVPQYGYLTLNNGSQAVGRSSFYCLEYFPSQMLRTGNNFQFSYTFEDVPFH
SSYAHSQSLDRLMNPLIDQYLYYLNRTQGTTSGTTNQSRLLFSQAGPQSMSLQARNWLPGPCYR
QQRLSKTANDNNNSNFPWTAASKYHLNGRDSLVNPGPAMASHKDDEEKFFPMHGNLIFGKEGTT
ASNAELDNVMITDEEEIRTTNPVATEQYGTVANNLQSSNTAPTTRTVNDQGALPGMVWQDRDVY
LQGPIWAKIPHTDGHFHPSPLMGGFGLKHPPPQIMIKNTPVPANPPTTFSPAKFASFITQYSTG
QVSVEIEWELQKENSKRWNPEIQYTSNYNKSVNVDFTVDTNGVYSEPRPIGTRYLTRPL
LK03-X44:
pLK03-T492V (SEQ ID NO: 27)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKVANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X45:
pLK03-T663V (SEQ ID NO: 28)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFVPAKFASFITQYSTGQVSVEIEWELQKENSKRWNPEIQYTSN
YNKSVNVDFTVDTNGVYSEPRPIGTRYLTRPL
LK03-X46:
pLK03-T492V-T663V (SEQ ID NO: 29)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKVANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFVPAKFASFITQYSTGQVSVEIEWELQKENSKRWNPEIQYTSN
YNKSVNVDFTVDTNGVYSEPRPIGTRYLTRPL
LK03-X47:
pLK03-K137R-T492V (SEQ ID NO: 30)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAARTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKVANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X48:
pLK03-K137R-T663V (SEQ ID NO: 31)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAARTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFVPAKFASFITQYSTGQVSVEIEWELQKENSKRWNPEIQYTSN
YNKSVNVDFTVDTNGVYSEPRPIGTRYLTRPL
LK03-X49
pLK03-K137R-T492V-T663V (SEQ ID NO: 32)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAARTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKVANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFVPAKFASFITQYSTGQVSVEIEWELQKENSKRWNPEIQYTSN
YNKSVNVDFTVDTNGVYSEPRPIGTRYLTRPL
LK03-X50:
pLK03-Y50A (SEQ ID NO: 33)
MAADGYLPDWLEDNLSEGIREWWALQPGAPKPKANQQHQDNARGLVLPGAKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X51:
pLK03-Y50F(SEQ ID NO: 34)
MAADGYLPDWLEDNLSEGIREWWALQPGAPKPKANQQHQDNARGLVLPGFKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X52:
pLK03-Y50E(SEQ ID NO: 35)
MAADGYLPDWLEDNLSEGIREWWALQPGAPKPKANQQHQDNARGLVLPGEKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X53:
pLK03-Y252A (SEQ ID NO: 36)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTANNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X54:
pLK03-Y252F (SEQ ID NO: 37)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTFNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X55:
pLK03-L171A (SEQ ID NO: 38)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRANFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X56:
pLK03-L479A (SEQ ID NO: 39)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWAPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X57:
pLK03-L517A (SEQ ID NO: 40)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSAVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL
LK03-X58:
pLK03-N691A (SEQ ID NO: 41)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKEASKRWNPEIQYTSN
YNKSVNVDFTVDTNGVYSEPRPIGTRYLTRPL
LK03-X60:
LK03-140hnRNP_M_NLS (SEQ ID NO: 42)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAGEGERPAQNEKRKEKNIKRGGNRFEPYPGKKRPVDQSPQEPDSSSGVGKSGKQ
PARKRLNFGQTGDSESVPDPQPLGEPPAAPTSLGSNTMASGGGAPMADNNEGADGVGNSSGNWH
CDSQWLGDRVITTSTRTWALPTYNNHLYKQISSQSGASNDNHYFGYSTPWGYFDFNRFHCHFSP
RDWQRLINNNWGFRPKKLSFKLFNIQVKEVTQNDGTTTIANNLTSTVQVFTDSEYQLPYVLGSA
HQGCLPPFPADVFMVPQYGYLTLNNGSQAVGRSSFYCLEYFPSQMLRTGNNFQFSYTFEDVPFH
SSYAHSQSLDRLMNPLIDQYLYYLNRTQGTTSGTTNQSRLLFSQAGPQSMSLQARNWLPGPCYR
QQRLSKTANDNNNSNFPWTAASKYHLNGRDSLVNPGPAMASHKDDEEKFFPMHGNLIFGKEGTT
ASNAELDNVMITDEEEIRTTNPVATEQYGTVANNLQSSNTAPTTRTVNDQGALPGMVWQDRDVY
LQGPIWAKIPHTDGHFHPSPLMGGFGLKHPPPQIMIKNTPVPANPPTTFSPAKFASFITQYSTG
QVSVEIEWELQKENSKRWNPEIQYTSNYNKSVNVDFTVDTNGVYSEPRPIGTRYLTRPL
LK03-X61:
LK03-140-ptHRP_NLS (SEQ ID NO: 43)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTARYLTQETNKVETYKEQPLKTPGKKKKGKPPGKKRPVDQSPQEPDSSSGVGKSG
KQPARKRLNFGQTGDSESVPDPQPLGEPPAAPTSLGSNTMASGGGAPMADNNEGADGVGNSSGN
WHCDSQWLGDRVITTSTRTWALPTYNNHLYKQISSQSGASNDNHYFGYSTPWGYFDFNRFHCHF
SPRDWQRLINNNWGFRPKKLSFKLFNIQVKEVTQNDGTTTIANNLTSTVQVFTDSEYQLPYVLG
SAHQGCLPPFPADVFMVPQYGYLTLNNGSQAVGRSSFYCLEYFPSQMLRTGNNFQFSYTFEDVP
FHSSYAHSQSLDRLMNPLIDQYLYYLNRTQGTTSGTTNQSRLLFSQAGPQSMSLQARNWLPGPC
YRQQRLSKTANDNNNSNFPWTAASKYHLNGRDSLVNPGPAMASHKDDEEKFFPMHGNLIFGKEG
TTASNAELDNVMITDEEEIRTTNPVATEQYGTVANNLQSSNTAPTTRTVNDQGALPGMVWQDRD
VYLQGPIWAKIPHTDGHFHPSPLMGGFGLKHPPPQIMIKNTPVPANPPTTFSPAKFASFITQYS
TGQVSVEIEWELQKENSKRWNPEIQYTSNYNKSVNVDFTVDTNGVYSEPRPIGTRYLTRPL
LK03-X62
LK03-140-class2_NLS (SEQ ID NO: 44)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTARAAKRPRTTPGKKRPVDQSPQEPDSSSGVGKSGKQPARKRLNFGQTGDSESVP
DPQPLGEPPAAPTSLGSNTMASGGGAPMADNNEGADGVGNSSGNWHCDSQWLGDRVITTSTRTW
ALPTYNNHLYKQISSQSGASNDNHYFGYSTPWGYFDFNRFHCHFSPRDWQRLINNNWGFRPKKL
SFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFQFSYTFEDVPFHSSYAHSQSLDRLMNPLID
QYLYYLNRTQGTTSGTTNQSRLLFSQAGPQSMSLQARNWLPGPCYRQQRLSKTANDNNNSNFPW
TAASKYHLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHP
SPLMGGFGLKHPPPQIMIKNTPVPANPPTTFSPAKFASFITQYSTGQVSVEIEWELQKENSKRW
NPEIQYTSNYNKSVNVDFTVDTNGVYSEPRPIGTRYLTRPL
LK03-X63:
LK03-140-class3_NLS (SEQ ID NO: 45)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAAAAKRSWSAAFPGKKRPVDQSPQEPDSSSGVGKSGKQPARKRLNFGQTGDSES
VPDPQPLGEPPAAPTSLGSNTMASGGGAPMADNNEGADGVGNSSGNWHCDSQWLGDRVITTSTR
TWALPTYNNHLYKQISSQSGASNDNHYFGYSTPWGYFDFNRFHCHFSPRDWQRLINNNWGFRPK
KLSFKLFNIQVKEVTQNDGTTTIANNLTSTVQVFTDSEYQLPYVLGSAHQGCLPPFPADVFMVP
QYGYLTLNNGSQAVGRSSFYCLEYFPSQMLRTGNNFQFSYTFEDVPFHSSYAHSQSLDRLMNPL
IDQYLYYLNRTQGTTSGTTNQSRLLFSQAGPQSMSLQARNWLPGPCYRQQRLSKTANDNNNSNF
PWTAASKYHLNGRDSLVNPGPAMASHKDDEEKFFPMHGNLIFGKEGTTASNAELDNVMITDEEE
IRTTNPVATEQYGTVANNLQSSNTAPTTRTVNDQGALPGMVWQDRDVYLQGPIWAKIPHTDGHF
HPSPLMGGFGLKHPPPQIMIKNTPVPANPPTTFSPAKFASFITQYSTGQVSVEIEWELQKENSK
RWNPEIQYTSNYNKSVNVDFTVDTNGVYSEPRPIGTRYLTRPL
LK03-X64:
LK03-K137R-140-cmycNLS (SEQ ID NO: 46)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAARTAPAAKRVKLDPGKKRPVDQSPQEPDSSSGVGKSGKQPARKRLNFGQTGDSESVP
DPQPLGEPPAAPTSLGSNTMASGGGAPMADNNEGADGVGNSSGNWHCDSQWLGDRVITTSTRTW
ALPTYNNHLYKQISSQSGASNDNHYFGYSTPWGYFDFNRFHCHFSPRDWQRLINNNWGFRPKKL
SFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFQFSYTFEDVPFHSSYAHSQSLDRLMNPLID
QYLYYLNRTQGTTSGTTNQSRLLFSQAGPQSMSLQARNWLPGPCYRQQRLSKTANDNNNSNFPW
TAASKYHLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHP
SPLMGGFGLKHPPPQIMIKNTPVPANPPTTFSPAKFASFITQYSTGQVSVEIEWELQKENSKRW
NPEIQYTSNYNKSVNVDFTVDTNGVYSEPRPIGTRYLTRPL
LK03-X65:
pLK03-35_GLFDIIKKIAESF (SEQ ID NO: 47)
MAADGYLPDWLEDNLSEGIREWWALQPGAPKPKAGLFDIIKKIAESFNQQHQDNARGLVLPGYK
YLGPGNGLDKGEPVNAADAAALEHDKAYDQQLKAGDNPYLKYNHADAEFQERLKEDTSFGGNLG
RAVFQAKKRLLEPLGLVEEAAKTAPGKKRPVDQSPQEPDSSSGVGKSGKQPARKRLNFGQTGDS
ESVPDPQPLGEPPAAPTSLGSNTMASGGGAPMADNNEGADGVGNSSGNWHCDSQWLGDRVIITS
TRTWALPTYNNHLYKQISSQSGASNDNHYFGYSTPWGYFDFNRFHCHFSPRDWQRLINNNWGFR
PKKLSFKLFNIQVKEVTQNDGTTTIANNLTSTVQVFTDSEYQLPYVLGSAHQGCLPPFPADVFM
VPQYGYLTLNNGSQAVGRSSFYCLEYFPSQMLRTGNNFQFSYTFEDVPFHSSYAHSQSLDRLMN
PLIDQYLYYLNRTQGTTSGTTNQSRLLFSQAGPQSMSLQARNWLPGPCYRQQRLSKTANDNNNS
NFPWTAASKYHLNGRDSLVNPGPAMASHKDDEEKFFPMHGNLIFGKEGTTASNAELDNVMIIDE
EEIRTTNPVATEQYGTVANNLQSSNTAPTTRTVNDQGALPGMVWQDRDVYLQGPIWAKIPHTDG
HFHPSPLMGGFGLKHPPPQIMIKNTPVPANPPTTFSPAKFASFITQYSTGQVSVEIEWELQKEN
SKRWNPEIQYTSNYNKSVNVDFTVDTNGVYSEPRPIGTRYLTRPL
LK03-X66:
pLK03-35_FFLIPKG (SEQ ID NO: 48)
MAADGYLPDWLEDNLSEGIREWWALQPGAPKPKAFFLIPKGNQQHQDNARGLVLPGYKYLGPGN
GLDKGEPVNAADAAALEHDKAYDQQLKAGDNPYLKYNHADAEFQERLKEDTSFGGNLGRAVFQA
KKRLLEPLGLVEEAAKTAPGKKRPVDQSPQEPDSSSGVGKSGKQPARKRLNFGQTGDSESVPDP
QPLGEPPAAPTSLGSNTMASGGGAPMADNNEGADGVGNSSGNWHCDSQWLGDRVITTSTRTWAL
PTYNNHLYKQISSQSGASNDNHYFGYSTPWGYFDFNRFHCHFSPRDWQRLINNNWGFRPKKLSF
KLFNIQVKEVTQNDGTTTIANNLTSTVQVFTDSEYQLPYVLGSAHQGCLPPFPADVFMVPQYGY
LTLNNGSQAVGRSSFYCLEYFPSQMLRTGNNFQFSYTFEDVPFHSSYAHSQSLDRLMNPLIDQY
LYYLNRTQGTTSGTTNQSRLLFSQAGPQSMSLQARNWLPGPCYRQQRLSKTANDNNNSNFPWTA
ASKYHLNGRDSLVNPGPAMASHKDDEEKFFPMHGNLIFGKEGTTASNAELDNVMITDEEEIRTT
NPVATEQYGTVANNLQSSNTAPTTRTVNDQGALPGMVWQDRDVYLQGPIWAKIPHTDGHFHPSP
LMGGFGLKHPPPQIMIKNTPVPANPPTTFSPAKFASFITQYSTGQVSVEIEWELQKENSKRWNP
EIQYTSNYNKSVNVDFTVDTNGVYSEPRPIGTRYLTRPL
LK03-X67:
pLK03-35_transportan (SEQ ID NO: 49)
MAADGYLPDWLEDNLSEGIREWWALQPGAPKPKAGWTLNSAGYLLGKINLKALAALAKKILNQQ
HQDNARGLVLPGYKYLGPGNGLDKGEPVNAADAAALEHDKAYDQQLKAGDNPYLKYNHADAEFQ
ERLKEDTSFGGNLGRAVFQAKKRLLEPLGLVEEAAKTAPGKKRPVDQSPQEPDSSSGVGKSGKQ
PARKRLNFGQTGDSESVPDPQPLGEPPAAPTSLGSNTMASGGGAPMADNNEGADGVGNSSGNWH
CDSQWLGDRVITTSTRTWALPTYNNHLYKQISSQSGASNDNHYFGYSTPWGYFDFNRFHCHFSP
RDWQRLINNNWGFRPKKLSFKLFNIQVKEVTQNDGTTTIANNLTSTVQVFTDSEYQLPYVLGSA
HQGCLPPFPADVFMVPQYGYLTLNNGSQAVGRSSFYCLEYFPSQMLRTGNNFQFSYTFEDVPFH
SSYAHSQSLDRLMNPLIDQYLYYLNRTQGTTSGTTNQSRLLFSQAGPQSMSLQARNWLPGPCYR
QQRLSKTANDNNNSNFPWTAASKYHLNGRDSLVNPGPAMASHKDDEEKFFPMHGNLIFGKEGTT
ASNAELDNVMITDEEEIRTTNPVATEQYGTVANNLQSSNTAPTTRTVNDQGALPGMVWQDRDVY
LQGPIWAKIPHTDGHFHPSPLMGGFGLKHPPPQIMIKNTPVPANPPTTFSPAKFASFITQYSTG
QVSVEIEWELQKENSKRWNPEIQYTSNYNKSVNVDFTVDTNGVYSEPRPIGTRYLTRPL
LK03-X68:
pLK03-35_TAT(SEQ ID NO: 50)
MAADGYLPDWLEDNLSEGIREWWALQPGAPKPKAGRKKRRQRRRPPQNQQHQDNARGLVLPGYK
YLGPGNGLDKGEPVNAADAAALEHDKAYDQQLKAGDNPYLKYNHADAEFQERLKEDTSFGGNLG
RAVFQAKKRLLEPLGLVEEAAKTAPGKKRPVDQSPQEPDSSSGVGKSGKQPARKRLNFGQTGDS
ESVPDPQPLGEPPAAPTSLGSNTMASGGGAPMADNNEGADGVGNSSGNWHCDSQWLGDRVIITS
TRTWALPTYNNHLYKQISSQSGASNDNHYFGYSTPWGYFDFNRFHCHFSPRDWQRLINNNWGFR
PKKLSFKLFNIQVKEVTQNDGTTTIANNLTSTVQVFTDSEYQLPYVLGSAHQGCLPPFPADVFM
VPQYGYLTLNNGSQAVGRSSFYCLEYFPSQMLRTGNNFQFSYTFEDVPFHSSYAHSQSLDRLMN
PLIDQYLYYLNRTQGTTSGTTNQSRLLFSQAGPQSMSLQARNWLPGPCYRQQRLSKTANDNNNS
NFPWTAASKYHLNGRDSLVNPGPAMASHKDDEEKFFPMHGNLIFGKEGTTASNAELDNVMIIDE
EEIRTTNPVATEQYGTVANNLQSSNTAPTTRTVNDQGALPGMVWQDRDVYLQGPIWAKIPHTDG
HFHPSPLMGGFGLKHPPPQIMIKNTPVPANPPTTFSPAKFASFITQYSTGQVSVEIEWELQKEN
SKRWNPEIQYTSNYNKSVNVDFTVDTNGVYSEPRPIGTRYLTRPL
LK03-X69:
pLK03-35_KKAAAVLLPVLLAAP (SEQ ID NO: 51)
MAADGYLPDWLEDNLSEGIREWWALQPGAPKPKAKKAAAVLLPVLLAAPNQQHQDNARGLVLPG
YKYLGPGNGLDKGEPVNAADAAALEHDKAYDQQLKAGDNPYLKYNHADAEFQERLKEDTSFGGN
LGRAVFQAKKRLLEPLGLVEEAAKTAPGKKRPVDQSPQEPDSSSGVGKSGKQPARKRLNFGQTG
DSESVPDPQPLGEPPAAPTSLGSNTMASGGGAPMADNNEGADGVGNSSGNWHCDSQWLGDRVIT
TSTRTWALPTYNNHLYKQISSQSGASNDNHYFGYSTPWGYFDFNRFHCHFSPRDWQRLINNNWG
FRPKKLSFKLFNIQVKEVTQNDGTTTIANNLTSTVQVFTDSEYQLPYVLGSAHQGCLPPFPADV
FMVPQYGYLTLNNGSQAVGRSSFYCLEYFPSQMLRTGNNFQFSYTFEDVPFHSSYAHSQSLDRL
MNPLIDQYLYYLNRTQGTTSGTTNQSRLLFSQAGPQSMSLQARNWLPGPCYRQQRLSKTANDNN
NSNFPWTAASKYHLNGRDSLVNPGPAMASHKDDEEKFFPMHGNLIFGKEGTTASNAELDNVMIT
DEEEIRTTNPVATEQYGTVANNLQSSNTAPTTRTVNDQGALPGMVWQDRDVYLQGPIWAKIPHT
DGHFHPSPLMGGFGLKHPPPQIMIKNTPVPANPPTTFSPAKFASFITQYSTGQVSVEIEWELQK
ENSKRWNPEIQYTSNYNKSVNVDFTVDTNGVYSEPRPIGTRYLTRPL
LK03-X70:
pLK03-35_penetratinML(SEQ ID NO: 52)
MAADGYLPDWLEDNLSEGIREWWALQPGAPKPKARQIKIWFQNRRLKWKKGGNQQHQDNARGLV
LPGYKYLGPGNGLDKGEPVNAADAAALEHDKAYDQQLKAGDNPYLKYNHADAEFQERLKEDTSF
GGNLGRAVFQAKKRLLEPLGLVEEAAKTAPGKKRPVDQSPQEPDSSSGVGKSGKQPARKRLNFG
QTGDSESVPDPQPLGEPPAAPTSLGSNTMASGGGAPMADNNEGADGVGNSSGNWHCDSQWLGDR
VITTSTRTWALPTYNNHLYKQISSQSGASNDNHYFGYSTPWGYFDFNRFHCHFSPRDWQRLINN
NWGFRPKKLSFKLFNIQVKEVTQNDGTTTIANNLTSTVQVFTDSEYQLPYVLGSAHQGCLPPFP
ADVFMVPQYGYLTLNNGSQAVGRSSFYCLEYFPSQMLRTGNNFQFSYTFEDVPFHSSYAHSQSL
DRLMNPLIDQYLYYLNRTQGTTSGTTNQSRLLFSQAGPQSMSLQARNWLPGPCYRQQRLSKTAN
DNNNSNFPWTAASKYHLNGRDSLVNPGPAMASHKDDEEKFFPMHGNLIFGKEGTTASNAELDNV
MITDEEEIRTTNPVATEQYGTVANNLQSSNTAPTTRTVNDQGALPGMVWQDRDVYLQGPIWAKI
PHTDGHFHPSPLMGGFGLKHPPPQIMIKNTPVPANPPTTFSPAKFASFITQYSTGQVSVEIEWE
LQKENSKRWNPEIQYTSNYNKSVNVDFTVDTNGVYSEPRPIGTRYLTRPL
LK03-X71:
pLK03-35_R16 (SEQ ID NO: 53)
MAADGYLPDWLEDNLSEGIREWWALQPGAPKPKARRRRRRRRRRRRRRRRNQQHQDNARGLVLP
GYKYLGPGNGLDKGEPVNAADAAALEHDKAYDQQLKAGDNPYLKYNHADAEFQERLKEDTSFGG
NLGRAVFQAKKRLLEPLGLVEEAAKTAPGKKRPVDQSPQEPDSSSGVGKSGKQPARKRLNFGQT
GDSESVPDPQPLGEPPAAPTSLGSNTMASGGGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNW
GFRPKKLSFKLFNIQVKEVTQNDGTTTIANNLTSTVQVFTDSEYQLPYVLGSAHQGCLPPFPAD
VFMVPQYGYLTLNNGSQAVGRSSFYCLEYFPSQMLRTGNNFQFSYTFEDVPFHSSYAHSQSLDR
LMNPLIDQYLYYLNRTQGTTSGTTNQSRLLFSQAGPQSMSLQARNWLPGPCYRQQRLSKTANDN
NNSNFPWTAASKYHLNGRDSLVNPGPAMASHKDDEEKFFPMHGNLIFGKEGTTASNAELDNVMI
TDEEEIRTTNPVATEQYGTVANNLQSSNTAPTTRTVNDQGALPGMVWQDRDVYLQGPIWAKIPH
TDGHFHPSPLMGGFGLKHPPPQIMIKNTPVPANPPTTFSPAKFASFITQYSTGQVSVEIEWELQ
KENSKRWNPEIQYTSNYNKSVNVDFTVDTNGVYSEPRPIGTRYLTRPL
LK03-X72:
pLK03-35_K16 (SEQ ID NO: 54)
MAADGYLPDWLEDNLSEGIREWWALQPGAPKPKAKKKKKKKKKKKKKKKKNQQHQDNARGLVLP
GYKYLGPGNGLDKGEPVNAADAAALEHDKAYDQQLKAGDNPYLKYNHADAEFQERLKEDTSFGG
NLGRAVFQAKKRLLEPLGLVEEAAKTAPGKKRPVDQSPQEPDSSSGVGKSGKQPARKRLNFGQT
GDSESVPDPQPLGEPPAAPTSLGSNTMASGGGAPMADNNEGADGVGNSSGNWHCDSQWLGDRVI
TTSTRTWALPTYNNHLYKQISSQSGASNDNHYFGYSTPWGYFDFNRFHCHFSPRDWQRLINNNW
GFRPKKLSFKLFNIQVKEVTQNDGTTTIANNLTSTVQVFTDSEYQLPYVLGSAHQGCLPPFPAD
VFMVPQYGYLTLNNGSQAVGRSSFYCLEYFPSQMLRTGNNFQFSYTFEDVPFHSSYAHSQSLDR
LMNPLIDQYLYYLNRTQGTTSGTTNQSRLLFSQAGPQSMSLQARNWLPGPCYRQQRLSKTANDN
NNSNFPWTAASKYHLNGRDSLVNPGPAMASHKDDEEKFFPMHGNLIFGKEGTTASNAELDNVMI
TDEEEIRTTNPVATEQYGTVANNLQSSNTAPTTRTVNDQGALPGMVWQDRDVYLQGPIWAKIPH
TDGHFHPSPLMGGFGLKHPPPQIMIKNTPVPANPPTTFSPAKFASFITQYSTGQVSVEIEWELQ
KENSKRWNPEIQYTSNYNKSVNVDFTVDTNGVYSEPRPIGTRYLTRPL
LK03-X73:
pLK03-35_R16_ApoE (SEQ ID NO: 55)
MAADGYLPDWLEDNLSEGIREWWALQPGAPKPKARRRRRRRRRRRRRRRRLRVRLASHLRKLRK
RLLRDANQQHQDNARGLVLPGYKYLGPGNGLDKGEPVNAADAAALEHDKAYDQQLKAGDNPYLK
YNHADAEFQERLKEDTSFGGNLGRAVFQAKKRLLEPLGLVEEAAKTAPGKKRPVDQSPQEPDSS
SGVGKSGKQPARKRLNFGQTGDSESVPDPQPLGEPPAAPTSLGSNTMASGGGAPMADNNEGADG
VGNSSGNWHCDSQWLGDRVITTSTRTWALPTYNNHLYKQISSQSGASNDNHYFGYSTPWGYFDF
NRFHCHFSPRDWQRLINNNWGFRPKKLSFKLFNIQVKEVTQNDGTTTIANNLTSTVQVFTDSEY
QLPYVLGSAHQGCLPPFPADVFMVPQYGYLTLNNGSQAVGRSSFYCLEYFPSQMLRTGNNFQFS
YTFEDVPFHSSYAHSQSLDRLMNPLIDQYLYYLNRTQGTTSGTTNQSRLLFSQAGPQSMSLQAR
NWLPGPCYRQQRLSKTANDNNNSNFPWTAASKYHLNGRDSLVNPGPAMASHKDDEEKFFPMHGN
LIFGKEGTTASNAELDNVMITDEEEIRTTNPVATEQYGTVANNLQSSNTAPTTRTVNDQGALPG
MVWQDRDVYLQGPIWAKIPHTDGHFHPSPLMGGFGLKHPPPQIMIKNTPVPANPPTTFSPAKFA
SFITQYSTGQVSVEIEWELQKENSKRWNPEIQYTSNYNKSVNVDFTVDTNGVYSEPRPIGTRYL
TRPL
LK03-X74:
LK03-L517A-140-PAAKRVKLD (SEQ ID NO: 56)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPAAKRVKLDPGKKRPVDQSPQEPDSSSGVGKSGKQPARKRLNFGQTGDSESVP
DPQPLGEPPAAPTSLGSNTMASGGGAPMADNNEGADGVGNSSGNWHCDSQWLGDRVITTSTRTW
ALPTYNNHLYKQISSQSGASNDNHYFGYSTPWGYFDFNRFHCHFSPRDWQRLINNNWGFRPKKL
SFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFQFSYTFEDVPFHSSYAHSQSLDRLMNPLID
QYLYYLNRTQGTTSGTTNQSRLLFSQAGPQSMSLQARNWLPGPCYRQQRLSKTANDNNNSNFPW
TAASKYHLNGRDSAVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIPHTDGHFHP
SPLMGGFGLKHPPPQIMIKNTPVPANPPTTFSPAKFASFITQYSTGQVSVEIEWELQKENSKRW
NPEIQYTSNYNKSVNVDFTVDTNGVYSEPRPIGTRYLTRPL
LK03-X75:
LK03-L517A-140hnRNP_D_NLS (SEQ ID NO: 57)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAYGDYSNQQSGYGKVSRRGGHQNSYKPYPGKKRPVDQSPQEPDSSSGVGKSGKQ
PARKRLNFGQTGDSESVPDPQPLGEPPAAPTSLGSNTMASGGGAPMADNNEGADGVGNSSGNWH
CDSQWLGDRVITTSTRTWALPTYNNHLYKQISSQSGASNDNHYFGYSTPWGYFDFNRFHCHFSP
RDWQRLINNNWGFRPKKLSFKLFNIQVKEVTQNDGTTTIANNLTSTVQVFTDSEYQLPYVLGSA
HQGCLPPFPADVFMVPQYGYLTLNNGSQAVGRSSFYCLEYFPSQMLRTGNNFQFSYTFEDVPFH
SSYAHSQSLDRLMNPLIDQYLYYLNRTQGTTSGTTNQSRLLFSQAGPQSMSLQARNWLPGPCYR
QQRLSKTANDNNNSNFPWTAASKYHLNGRDSAVNPGPAMASHKDDEEKFFPMHGNLIFGKEGTT
ASNAELDNVMITDEEEIRTTNPVATEQYGTVANNLQSSNTAPTTRTVNDQGALPGMVWQDRDVY
LQGPIWAKIPHTDGHFHPSPLMGGFGLKHPPPQIMIKNTPVPANPPTTFSPAKFASFITQYSTG
QVSVEIEWELQKENSKRWNPEIQYTSNYNKSVNVDFTVDTNGVYSEPRPIGTRYLTRPL
LK03-X76:
LK03-L517A-140-class3_NLS (SEQ ID NO: 58)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAAAAKRSWSAAFPGKKRPVDQSPQEPDSSSGVGKSGKQPARKRLNFGQTGDSES
VPDPQPLGEPPAAPTSLGSNTMASGGGAPMADNNEGADGVGNSSGNWHCDSQWLGDRVITTSTR
TWALPTYNNHLYKQISSQSGASNDNHYFGYSTPWGYFDFNRFHCHFSPRDWQRLINNNWGFRPK
KLSFKLFNIQVKEVTQNDGTTTIANNLTSTVQVFTDSEYQLPYVLGSAHQGCLPPFPADVFMVP
QYGYLTLNNGSQAVGRSSFYCLEYFPSQMLRTGNNFQFSYTFEDVPFHSSYAHSQSLDRLMNPL
IDQYLYYLNRTQGTTSGTTNQSRLLFSQAGPQSMSLQARNWLPGPCYRQQRLSKTANDNNNSNF
PWTAASKYHLNGRDSAVNPGPAMASHKDDEEKFFPMHGNLIFGKEGTTASNAELDNVMITDEEE
IRTTNPVATEQYGTVANNLQSSNTAPTTRTVNDQGALPGMVWQDRDVYLQGPIWAKIPHTDGHF
HPSPLMGGFGLKHPPPQIMIKNTPVPANPPTTFSPAKFASFITQYSTGQVSVEIEWELQKENSK
RWNPEIQYTSNYNKSVNVDFTVDTNGVYSEPRPIGTRYLTRPL

### Example 7

AMINO ACID SEQUENCES of SPARK100_(Spk100) and
SPARK100_(Spk100) VARIANTS

Spark100_X01:
Wild type Spark100 (SEQ ID NO: 59)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKKGQQPAKKRLNFGQTGDSESVPDPQPIGEP
PAAPSGVGPNTMAAGGGAPMADNNEGADGVGSSSGNWHCDSTWLGDRVITTSTRTWALPTYNNH
LYKQISNGTSGGSTNDNTYFGYSTPWGYFDFNRFHCHFSPRDWQRLINNNWGFRPKRLNFKLFN
IQVKEVTQNEGTKTIANNLTSTIQVFTDSEYQLPYVLGSAHQGCLPPFPADVFMIPQYGYLTLN
NGSQAVGRSSFYCLEYFPSQMLRTGNNFEFSYNFEDVPFHSSYAHSQSLDRLMNPLIDQYLYYL
SRTQSTGGTAGTQQLLFSQAGPNNMSAQAKNWLPGPCYRQQRVSTILSQNNNSNFAWTGATKYH
LNGRDSLVNPGVAMATHKDDEERFFPSSGVLMFGKQGAGKDNVDYSSVMLTSEEEIKTTNPVAT
EQYGVVADNLQQQNAAPIVGAVNSQGALPGMVWQNRDVYLQGPIWAKIPHTDGNFHPSPLMGGF
GLKHPPPQILIKNTPVPADPPTTFNQAKLASFITQYSTGQVSVEIEWELQKENSKRWNPEIQYT
SNYYKSTNVDFAVNTEGTYSEPRPIGTRYLTRNL
Spark100_X02:
Spark100_K137R (SEQ ID NO: 60)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVRTAPGKKRPVEPSPQRSPDSSTGIGKKGQQPAKKRLNFGQTGDSESVPDPQPIGEP
PAAPSGVGPNTMAAGGGAPMADNNEGADGVGSSSGNWHCDSTWLGDRVITTSTRTWALPTYNNH
LYKQISNGTSGGSTNDNTYFGYSTPWGYFDFNRFHCHFSPRDWQRLINNNWGFRPKRLNFKLFN
IQVKEVTQNEGTKTIANNLTSTIQVFTDSEYQLPYVLGSAHQGCLPPFPADVFMIPQYGYLTLN
NGSQAVGRSSFYCLEYFPSQMLRTGNNFEFSYNFEDVPFHSSYAHSQSLDRLMNPLIDQYLYYL
SRTQSTGGTAGTQQLLFSQAGPNNMSAQAKNWLPGPCYRQQRVSTTLSQNNNSNFAWTGATKYH
LNGRDSLVNPGVAMATHKDDEERFFPSSGVLMFGKQGAGKDNVDYSSVMLTSEEEIKTTNPVAT
EQYGVVADNLQQQNAAPIVGAVNSQGALPGMVWQNRDVYLQGPIWAKIPHTDGNFHPSPLMGGF
GLKHPPPQILIKNTPVPADPPTTFNQAKLASFITQYSTGQVSVEIEWELQKENSKRWNPEIQYT
SNYYKSTNVDFAVNTEGTYSEPRPIGTRYLTRNL
Spark100_X03:
Spark100_L519A (SEQ ID NO: 61)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKKGQQPAKKRLNFGQTGDSESVPDPQPIGEP
PAAPSGVGPNTMAAGGGAPMADNNEGADGVGSSSGNWHCDSTWLGDRVITTSTRTWALPTYNNH
LYKQISNGTSGGSTNDNTYFGYSTPWGYFDFNRFHCHFSPRDWQRLINNNWGFRPKRLNFKLFN
IQVKEVTQNEGTKTIANNLTSTIQVFTDSEYQLPYVLGSAHQGCLPPFPADVFMIPQYGYLTLN
NGSQAVGRSSFYCLEYFPSQMLRTGNNFEFSYNFEDVPFHSSYAHSQSLDRLMNPLIDQYLYYL
SRTQSTGGTAGTQQLLFSQAGPNNMSAQAKNWLPGPCYRQQRVSTILSQNNNSNFAWTGATKYH
LNGRDSAVNPGVAMATHKDDEERFFPSSGVLMFGKQGAGKDNVDYSSVMLTSEEEIKTTNPVAT
EQYGVVADNLQQQNAAPIVGAVNSQGALPGMVWQNRDVYLQGPIWAKIPHTDGNFHPSPLMGGF
GLKHPPPQILIKNTPVPADPPTTFNQAKLASFITQYSTGQVSVEIEWELQKENSKRWNPEIQYT
SNYYKSTNVDFAVNTEGTYSEPRPIGTRYLTRNL
Spark100_X04
pSpark100-140-cmycNLS (SEQ ID NO: 62)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPAAKRVKLDPGKKRPVEPSPQRSPDSSTGIGKKGQQPAKKRLNFGQTGDSESV
PDPQPIGEPPAAPSGVGPNTMAAGGGAPMADNNEGADGVGSSSGNWHCDSTWLGDRVITTSTRT
WALPTYNNHLYKQISNGTSGGSTNDNTYFGYSTPWGYFDFNRFHCHFSPRDWQRLINNNWGFRP
KRLNFKLFNIQVKEVTQNEGTKTIANNLTSTIQVFTDSEYQLPYVLGSAHQGCLPPFPADVFMI
PQYGYLTLNNGSQAVGRSSFYCLEYFPSQMLRTGNNFEFSYNFEDVPFHSSYAHSQSLDRLMNP
LIDQYLYYLSRTQSTGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKDNVDYSSVMLTSEEE
IKTTNPVATEQYGVVADNLQQQNAAPIVGAVNSQGALPGMVWQNRDVYLQGPIWAKIPHTDGNF
HPSPLMGGFGLKHPPPQILIKNTPVPADPPTTFNQAKLASFITQYSTGQVSVEIEWELQKENSK
RWNPEIQYTSNYYKSTNVDFAVNTEGTYSEPRPIGTRYLTRNL
Spark100_X05
pSpark100-140-hnRNP_D_NLS (SEQ ID NO: 63)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAYGDYSNQQSGYGKVSRRGGHQNSYKPYPGKKRPVEPSPQRSPDSSTGIGKKGQ
QPAKKRLNFGQTGDSESVPDPQPIGEPPAAPSGVGPNTMAAGGGAPMADNNEGADGVGSSSGNW
HCDSTWLGDRVITTSTRTWALPTYNNHLYKQISNGTSGGSTNDNTYFGYSTPWGYFDFNRFHCH
FSPRDWQRLINNNWGFRPKRLNFKLFNIQVKEVTQNEGTKTIANNLTSTIQVFTDSEYQLPYVL
GSAHQGCLPPFPADVFMIPQYGYLTLNNGSQAVGRSSFYCLEYFPSQMLRTGNNFEFSYNFEDV
PFHSSYAHSQSLDRLMNPLIDQYLYYLSRTQSTGGTAGTQQLLFSQAGPNNMSAQAKNWLPGPC
YRQQRVSTTLSQNNNSNFAWTGATKYHLNGRDSLVNPGVAMATHKDDEERFFPSSGVLMFGKQG
AGKDNVDYSSVMLTSEEEIKTTNPVATEQYGVVADNLQQQNAAPIVGAVNSQGALPGMVWQNRD
VYLQGPIWAKIPHTDGNFHPSPLMGGFGLKHPPPQILIKNTPVPADPPTTFNQAKLASFITQYS
TGQVSVEIEWELQKENSKRWNPEIQYTSNYYKSTNVDFAVNTEGTYSEPRPIGTRYLTRNL
Spark100_X06:
pSpark100-140-class3_NLS (SEQ ID NO: 64)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAAAAKRSWSAAFPGKKRPVEPSPQRSPDSSTGIGKKGQQPAKKRLNFGQTGDSE
SVPDPQPIGEPPAAPSGVGPNTMAAGGGAPMADNNEGADGVGSSSGNWHCDSTWLGDRVITTST
RTWALPTYNNHLYKQISNGTSGGSTNDNTYFGYSTPWGYFDFNRFHCHFSPRDWQRLINNNWGF
RPKRLNFKLFNIQVKEVTQNEGTKTIANNLTSTIQVFTDSEYQLPYVLGSAHQGCLPPFPADVF
MIPQYGYLTLNNGSQAVGRSSFYCLEYFPSQMLRTGNNFEFSYNFEDVPFHSSYAHSQSLDRLM
NPLIDQYLYYLSRTQSTGGTAGTQQLLFSQAGPNNMSAQAKNWLPGPCYRQQRVSTTLSQNNNS
NFAWTGATKYHLNGRDSLVNPGVAMATHKDDEERFFPSSGVLMFGKQGAGKDNVDYSSVMLTSE
EEIKTTNPVATEQYGVVADNLQQQNAAPIVGAVNSQGALPGMVWQNRDVYLQGPIWAKIPHTDG
NFHPSPLMGGFGLKHPPPQILIKNTPVPADPPTTFNQAKLASFITQYSTGQVSVEIEWELQKEN
SKRWNPEIQYTSNYYKSTNVDFAVNTEGTYSEPRPIGTRYLTRNL
Spark100_X07:
pSpark100-140-SV40_NLS (SEQ ID NO: 65)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPKKKRKVPGKKRPVEPSPQRSPDSSTGIGKKGQQPAKKRLNFGQTGDSESVPD
PQPIGEPPAAPSGVGPNTMAAGGGAPMADNNEGADGVGSSSGNWHCDSTWLGDRVITTSTRTWA
LPTYNNHLYKQISNGTSGGSTNDNTYFGYSTPWGYFDFNRFHCHFSPRDWQRLINNNWGFRPKR
LNFKLFNIQVKEVTQNEGTKTIANNLTSTIQVFTDSEYQLPYVLGSAHQGCLPPFPADVFMIPQ
YGYLTLNNGSQAVGRSSFYCLEYFPSQMLRTGNNFEFSYNFEDVPFHSSYAHSQSLDRLMNPLI
DQYLYYLSRTQSTGGTAGTQQLLFSQAGPNNMSAQAKNWLPGPCYRQQRVSTILSQNNNSNFAW
TGATKYHLNGRDSLVNPGVAMATHKDDEERFFPSSGVLMFGKQGAGKDNVDYSSVMLTSEEEIK
TTNPVATEQYGVVADNLQQQNAAPIVGAVNSQGALPGMVWQNRDVYLQGPIWAKIPHTDGNFHP
SPLMGGFGLKHPPPQILIKNTPVPADPPTTFNQAKLASFITQYSTGQVSVEIEWELQKENSKRW
NPEIQYTSNYYKSTNVDFAVNTEGTYSEPRPIGTRYLTRNL
Spark100_X08:
pSpark100-140-2x-cmycNLS (SEQ ID NO: 66)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPAAKRVKLDEDPAAKRVKLDPGKKRPVEPSPQRSPDSSTGIGKKGQQPAKKRL
NFGQTGDSESVPDPQPIGEPPAAPSGVGPNTMAAGGGAPMADNNEGADGVGSSSGNWHCDSTWL
GDRVITTSTRTWALPTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSEYQLPYVLGSAHQGC
LPPFPADVFMIPQYGYLTLNNGSQAVGRSSFYCLEYFPSQMLRTGNNFEFSYNFEDVPFHSSYA
HSQSLDRLMNPLIDQYLYYLSRTQSTGGTAGTQQLLFSQAGPNNMSAQAKNWLPGPCYRQQRVS
TTLSQNNNSNFAWTGATKYHLNGRDSLVNPGVAMATHKDDEERFFPSSGVLMFGKQGAGKDNVD
YSSVMLTSEEEIKTTNPVATEQYGVVADNLQQQNAAPIVGAVNSQGALPGMVWQNRDVYLQGPI
WAKIPHTDGNFHPSPLMGGFGLKHPPPQILIKNTPVPADPPTTFNQAKLASFITQYSTGQVSVE
IEWELQKENSKRWNPEIQYTSNYYKSTNVDFAVNTEGTYSEPRPIGTRYLTRNL
Spark100_X09
pSpark100-140-2x class3_NLS (SEQ ID NO: 67)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAAAAKRSWSAAFEDAAAKRSWSAAFPGKKRPVEPSPQRSPDSSTGIGKKGQQPA
KKRLNFGQTGDSESVPDPQPIGEPPAAPSGVGPNTMAAGGGAPMADNNEGADGVGSSSGNWHCD
STWLGDRVITTSTRTWALPTYNNHLYKQISNGTSGGSTNDNTYFGYSTPWGYFDFNRFHCHFSP
RDWQRLINNNWGFRPKRLNFKLFNIQVKEVTQNEGTKTIANNLTSTIQVFTDSEYQLPYVLGSA
HQGCLPPFPADVFMIPQYGYLTLNNGSQAVGRSSFYCLEYFPSQMLRTGNNFEFSYNFEDVPFH
SSYAHSQSLDRLMNPLIDQYLYYLSRTQSTGGTAGTQQLLFSQAGPNNMSAQAKNWLPGPCYRQ
QRVSTTLSQNNNSNFAWTGATKYHLNGRDSLVNPGVAMATHKDDEERFFPSSGVLMFGKQGAGK
DNVDYSSVMLTSEEEIKTTNPVATEQYGVVADNLQQQNAAPIVGAVNSQGALPGMVWQNRDVYL
QGPIWAKIPHTDGNFHPSPLMGGFGLKHPPPQILIKNTPVPADPPTTFNQAKLASFITQYSTGQ
VSVEIEWELQKENSKRWNPEIQYTSNYYKSTNVDFAVNTEGTYSEPRPIGTRYLTRNL
Spark100_X10:
pSpark100-140-2xSV40 NLS (SEQ ID NO: 68)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPKKKRKVEDPKKKRKVPGKKRPVEPSPQRSPDSSTGIGKKGQQPAKKRLNFGQ
TGDSESVPDPQPIGEPPAAPSGVGPNTMAAGGGAPMADNNEGADGVGSSSGNWHCDSTWLGDRV
ITTSTRTWALPTYNNHLYKQISNGTSGGSTNDNTYFGYSTPWGYFDFNRFHCHFSPRDWQRLIN
NNWGFRPKRLNFKLFNIQVKEVTQNEGTKTIANNLTSTIQVFTDSEYQLPYVLGSAHQGCLPPF
PADVFMIPQYGYLTLNNGSQAVGRSSFYCLEYFPSQMLRTGNNFEFSYNFEDVPFHSSYAHSQS
LDRLMNPLIDQYLYYLSRTQSTGGTAGTQQLLFSQAGPNNMSAQAKNWLPGPCYRQQRVSTTLS
QNNNSNFAWTGATKYHLNGRDSLVNPGVAMATHKDDEERFFPSSGVLMFGKQGAGKDNVDYSSV
MLTSEEEIKTTNPVATEQYGVVADNLQQQNAAPIVGAVNSQGALPGMVWQNRDVYLQGPIWAKI
PHTDGNFHPSPLMGGFGLKHPPPQILIKNTPVPADPPTTFNQAKLASFITQYSTGQVSVEIEWE
LQKENSKRWNPEIQYTSNYYKSTNVDFAVNTEGTYSEPRPIGTRYLTRNL
Spark100_X11:
pSpark100-140-1x class3_NLS-1x cmycNLS (SEQ ID NO: 69)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAAAAKRSWSAAFEDPAAKRVKLDPGKKRPVEPSPQRSPDSSTGIGKKGQQPAKK
RLNFGQTGDSESVPDPQPIGEPPAAPSGVGPNTMAAGGGAPMADNNEGADGVGSSSGNWHCDST
WLGDRVITTSTRTWALPTYNNHLYKQISNGTSGGSTNDNTYFGYSTPWGYFDFNRFHCHFSPRD
WQRLINNNWGFRPKRLNFKLFNIQVKEVTQNEGTKTIANNLTSTIQVFTDSEYQLPYVLGSAHQ
GCLPPFPADVFMIPQYGYLTLNNGSQAVGRSSFYCLEYFPSQMLRTGNNFEFSYNFEDVPFHSS
YAHSQSLDRLMNPLIDQYLYYLSRTQSTGGTAGTQQLLFSQAGPNNMSAQAKNWLPGPCYRQQR
VSTTLSQNNNSNFAWTGATKYHLNGRDSLVNPGVAMATHKDDEERFFPSSGVLMFGKQGAGKDN
VDYSSVMLTSEEEIKTTNPVATEQYGVVADNLQQQNAAPIVGAVNSQGALPGMVWQNRDVYLQG
PIWAKIPHTDGNFHPSPLMGGFGLKHPPPQILIKNTPVPADPPTTFNQAKLASFITQYSTGQVS
VEIEWELQKENSKRWNPEIQYTSNYYKSTNVDFAVNTEGTYSEPRPIGTRYLTRNL
Spark100_X12:
pSpark100-140-1x cmycNLS-1x class3_NLS (SEQ ID NO: 70)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPAAKRVKLDEDAAAKRSWSAAFPGKKRPVEPSPQRSPDSSTGIGKKGQQPAKK
RLNFGQTGDSESVPDPQPIGEPPAAPSGVGPNTMAAGGGAPMADNNEGADGVGSSSGNWHCDST
WLGDRVITTSTRTWALPTYNNHLYKQISNGTSGGSTNDNTYFGYSTPWGYFDFNRFHCHFSPRD
WQRLINNNWGFRPKRLNFKLFNIQVKEVTQNEGTKTIANNLTSTIQVFTDSEYQLPYVLGSAHQ
GCLPPFPADVFMIPQYGYLTLNNGSQAVGRSSFYCLEYFPSQMLRTGNNFEFSYNFEDVPFHSS
YAHSQSLDRLMNPLIDQYLYYLSRTQSTGGTAGTQQLLFSQAGPNNMSAQAKNWLPGPCYRQQR
VSTTLSQNNNSNFAWTGATKYHLNGRDSLVNPGVAMATHKDDEERFFPSSGVLMFGKQGAGKDN
VDYSSVMLTSEEEIKTTNPVATEQYGVVADNLQQQNAAPIVGAVNSQGALPGMVWQNRDVYLQG
PIWAKIPHTDGNFHPSPLMGGFGLKHPPPQILIKNTPVPADPPTTFNQAKLASFITQYSTGQVS
VEIEWELQKENSKRWNPEIQYTSNYYKSTNVDFAVNTEGTYSEPRPIGTRYLTRNL
Spark100_X13:
pSpark100-140-3xSV40 NLS (SEQ ID NO: 71)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPKKKRKVEDPKKKRKVEDPKKKRKVPGKKRPVEPSPQRSPDSSTGIGKKGQQP
AKKRLNFGQTGDSESVPDPQPIGEPPAAPSGVGPNTMAAGGGAPMADNNEGADGVGSSSGNWHC
DSTWLGDRVITTSTRTWALPTYNNHLYKQISNGTSGGSTNDNTYFGYSTPWGYFDFNRFHCHFS
PRDWQRLINNNWGFRPKRLNFKLFNIQVKEVTQNEGTKTIANNLTSTIQVFTDSEYQLPYVLGS
AHQGCLPPFPADVFMIPQYGYLTLNNGSQAVGRSSFYCLEYFPSQMLRTGNNFEFSYNFEDVPF
HSSYAHSQSLDRLMNPLIDQYLYYLSRTQSTGGTAGTQQLLFSQAGPNNMSAQAKNWLPGPCYR
QQRVSTTLSQNNNSNFAWTGATKYHLNGRDSLVNPGVAMATHKDDEERFFPSSGVLMFGKQGAG
KDNVDYSSVMLTSEEEIKTTNPVATEQYGVVADNLQQQNAAPIVGAVNSQGALPGMVWQNRDVY
LQGPIWAKIPHTDGNFHPSPLMGGFGLKHPPPQILIKNTPVPADPPTTFNQAKLASFITQYSTG
QVSVEIEWELQKENSKRWNPEIQYTSNYYKSTNVDFAVNTEGTYSEPRPIGTRYLTRNL
Spark100_X14:
pSpark100-140-3x-cmycNLS (SEQ ID NO: 72)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPAAKRVKLDEDPAAKRVKLDEDPAAKRVKLDPGKKRPVEPSPQRSPDSSTGIG
KKGQQPAKKRLNFGQTGDSESVPDPQPIGEPPAAPSGVGPNTMAAGGGAPMADNNEGADGVGSS
SGNWHCDSTWLGDRVITTSTRTWALPTYNNHLYKQISNGTSGGSTNDNTYFGYSTPWGYFDFNR
FHCHFSPRDWQRLINNNWGFRPKRLNFKLFNIQVKEVTQNEGTKTIANNLTSTIQVFTDSEYQL
PYVLGSAHQGCLPPFPADVFMIPQYGYLTLNNGSQAVGRSSFYCLEYFPSQMLRTGNNFEFSYN
FEDVPFHSSYAHSQSLDRLMNPLIDQYLYYLSRTQSTGGTAGTQQLLFSQAGPNNMSAQAKNWL
PGPCYRQQRVSTTLSQNNNSNFAWTGATKYHLNGRDSLVNPGVAMATHKDDEERFFPSSGVLMF
GKQGAGKDNVDYSSVMLTSEEEIKTTNPVATEQYGVVADNLQQQNAAPIVGAVNSQGALPGMVW
QNRDVYLQGPIWAKIPHTDGNFHPSPLMGGFGLKHPPPQILIKNTPVPADPPTTFNQAKLASFI
TQYSTGQVSVEIEWELQKENSKRWNPEIQYTSNYYKSTNVDFAVNTEGTYSEPRPIGTRYLTRN
L
Spark100_X15:
pSpark100-140-3x class3_NLS (SEQ ID NO: 73)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAAAAKRSWSAAFEDAAAKASWSAAFEDAAAKRSWSAAFPGKKRPVEPSPQRSPD
SSTGIGKKGQQPAKKRLNFGQTGDSESVPDPQPIGEPPAAPSGVGPNTMAAGGGAPMADNNEGA
DGVGSSSGNWHCDSTWLGDRVITTSTRTWALPTYNNHLYKQISNGTSGGSTNDNTYFGYSTPWG
YFDFNRFHCHFSPRDWQRLINNNWGFRPKRLNFKLFNIQVKEVTQNEGTKTIANNLTSTIQVFT
DSEYQLPYVLGSAHQGCLPPFPADVFMIPQYGYLTLNNGSQAVGRSSFYCLEYFPSQMLRTGNN
FEFSYNFEDVPFHSSYAHSQSLDRLMNPLIDQYLYYLSRTQSTGGTAGTQQLLFSQAGPNNMSA
QAKNWLPGPCYRQQRVSTTLSQNNNSNFAWTGATKYHLNGRDSLVNPGVAMATHKDDEERFFPS
SGVLMFGKQGAGKDNVDYSSVMLTSEEEIKTTNPVATEQYGVVADNLQQQNAAPIVGAVNSQGA
LPGMVWQNRDVYLQGPIWAKIPHTDGNFHPSPLMGGFGLKHPPPQILIKNTPVPADPPTTFNQA
KLASFITQYSTGQVSVEIEWELQKENSKRWNPEIQYTSNYYKSTNVDFAVNTEGTYSEPRPIGT
RYLTRNL
Spark100_X16:
pSpark100-140-2xcmycNLS-1x class3_NLS (SEQ ID NO: 74)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPAAKRVKLDEDPAAKRVKLDEDAAAKRSWSAAFPGKKRPVEPSPQRSPDSSTG
IGKKGQQPAKKRLNFGQTGDSESVPDPQPIGEPPAAPSGVGPNTMAAGGGAPMADNNEGADGVG
SSSGNWHCDSTWLGDRVITTSTRTWALPTYNNHLYKQISNGTSGGSTNDNTYFGYSTPWGYFDF
NRFHCHFSPRDWQRLINNNWGFRPKRLNFKLFNIQVKEVTQNEGTKTIANNLTSTIQVFTDSEY
QLPYVLGSAHQGCLPPFPADVFMIPQYGYLTLNNGSQAVGRSSFYCLEYFPSQMLRTGNNFEFS
YNFEDVPFHSSYAHSQSLDRLMNPLIDQYLYYLSRTQSTGGTAGTQQLLFSQAGPNNMSAQAKN
WLPGPCYRQQRVSTTLSQNNNSNFAWTGATKYHLNGRDSLVNPGVAMATHKDDEERFFPSSGVL
MFGKQGAGKDNVDYSSVMLTSEEEIKTTNPVATEQYGVVADNLQQQNAAPIVGAVNSQGALPGM
VWQNRDVYLQGPIWAKIPHTDGNFHPSPLMGGFGLKHPPPQILIKNTPVPADPPTTFNQAKLAS
FITQYSTGQVSVEIEWELQKENSKRWNPEIQYTSNYYKSTNVDFAVNTEGTYSEPRPIGTRYLT
RNL
Spark100_X17:
pSpark100-140-2x class3_NLS-1x cmycNLS (SEQ ID NO: 75)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAAAAKRSWSAAFEDAAAKRSWSAAFEDPAAKRVKLDPGKKRPVEPSPQRSPDSS
TGIGKKGQQPAKKRLNFGQTGDSESVPDPQPIGEPPAAPSGVGPNTMAAGGGAPMADNNEGADG
VGSSSGNWHCDSTWLGDRVITTSTRTWALPTYNNHLYKQISNGTSGGSTNDNTYFGYSTPWGYF
DFNRFHCHFSPRDWQRLINNNWGFRPKRLNFKLFNIQVKEVTQNEGTKTIANNLTSTIQVFTDS
EYQLPYVLGSAHQGCLPPFPADVFMIPQYGYLTLNNGSQAVGRSSFYCLEYFPSQMLRTGNNFE
FSYNFEDVPFHSSYAHSQSLDRLMNPLIDQYLYYLSRTQSTGGTAGTQQLLFSQAGPNNMSAQA
KNWLPGPCYRQQRVSTTLSQNNNSNFAWTGATKYHLNGRDSLVNPGVAMATHKDDEERFFPSSG
VLMFGKQGAGKDNVDYSSVMLTSEEEIKTTNPVATEQYGVVADNLQQQNAAPIVGAVNSQGALP
GMVWQNRDVYLQGPIWAKIPHTDGNFHPSPLMGGFGLKHPPPQILIKNTPVPADPPTTFNQAKL
ASFITQYSTGQVSVEIEWELQKENSKRWNPEIQYTSNYYKSTNVDFAVNTEGTYSEPRPIGTRY
LTRNL
Spark100_X18:
pSpark100-140-1x hnRNP_D_NLS-1xclass3NLS-1x cmycNLS
(SEQ ID NO: 76)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAYGDYSNQQSGYGKVSRRGGHQNSYKPYEDAAAKRSWSAAFEDPAAKRVKLDPG
KKRPVEPSPQRSPDSSTGIGKKGQQPAKKRLNFGQTGDSESVPDPQPIGEPPAAPSGVGPNTMA
AGGGAPMADNNEGADGVGSSSGNWHCDSTWLGDRVITTSTRTWALPTYNNHLYKQISNGTSGGS
TNDNTYFGYSTPWGYFDFNRFHCHFSPRDWQRLINNNWGFRPKRLNFKLFNIQVKEVTQNEGTK
TIANNLTSTIQVFTDSEYQLPYVLGSAHQGCLPPFPADVFMIPQYGYLTLNNGSQAVGRSSFYC
LEYFPSQMLRTGNNFEFSYNFEDVPFHSSYAHSQSLDRLMNPLIDQYLYYLSRTQSTGGTAGTQ
QLLFSQAGPNNMSAQAKNWLPGPCYRQQRVSTILSQNNNSNFAWTGATKYHLNGRDSLVNPGVA
MATHKDDEERFFPSSGVLMFGKQGAGKDNVDYSSVMLTSEEEIKTTNPVATEQYGVVADNLQQQ
NAAPIVGAVNSQGALPGMVWQNRDVYLQGPIWAKIPHTDGNFHPSPLMGGFGLKHPPPQILIKN
TPVPADPPTTFNQAKLASFITQYSTGQVSVEIEWELQKENSKRWNPEIQYTSNYYKSTNVDFAV
NTEGTYSEPRPIGTRYLTRNL
Spark100_X19:
pSpark100_L519A-140-cmycNLS (SEQ ID NO: 77)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPAAKRVKLDPGKKRPVEPSPQRSPDSSTGIGKKGQQPAKKRLNFGQTGDSESV
PDPQPIGEPPAAPSGVGPNTMAAGGGAPMADNNEGADGVGSSSGNWHCDSTWLGDRVITTSTRT
WALPTYNNHLYKQISNGTSGGSTNDNTYFGYSTPWGYFDFNRFHCHFSPRDWQRLINNNWGFRP
KRLNFKLFNIQVKEVTQNEGTKTIANNLTSTIQVFTDSEYQLPYVLGSAHQGCLPPFPADVFMI
PQYGYLTLNNGSQAVGRSSFYCLEYFPSQMLRTGNNFEFSYNFEDVPFHSSYAHSQSLDRLMNP
LIDQYLYYLSRTQSTGGTAGTQQLLFSQAGPNNMSAQAKNWLPGPCYRQQRVSTTLSQNNNSNF
AWTGATKYHLNGRDSAVNPGVAMATHKDDEERFFPSSGVLMFGKQGAGKDNVDYSSVMLTSEEE
IKTTNPVATEQYGVVADNLQQQNAAPIVGAVNSQGALPGMVWQNRDVYLQGPIWAKIPHTDGNF
HPSPLMGGFGLKHPPPQILIKNTPVPADPPTTFNQAKLASFITQYSTGQVSVEIEWELQKENSK
RWNPEIQYTSNYYKSTNVDFAVNTEGTYSEPRPIGTRYLTRNL
Spark100_X20
pSpark100-163-cmycNLS (SEQ ID NO: 78)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKPAAKRVKLDKGQQPAKKRLNFGQTGDSESV
PDPQPIGEPPAAPSGVGPNTMAAGGGAPMADNNEGADGVGSSSGNWHCDSTWLGDRVITTSTRT
WALPTYNNHLYKQISNGTSGGSTNDNTYFGYSTPWGYFDFNRFHCHFSPRDWQRLINNNWGFRP
KRLNFKLFNIQVKEVTQNEGTKTIANNLTSTIQVFTDSEYQLPYVLGSAHQGCLPPFPADVFMI
PQYGYLTLNNGSQAVGRSSFYCLEYFPSQMLRTGNNFEFSYNFEDVPFHSSYAHSQSLDRLMNP
LIDQYLYYLSRTQSTGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKDNVDYSSVMLTSEEE
IKTTNPVATEQYGVVADNLQQQNAAPIVGAVNSQGALPGMVWQNRDVYLQGPIWAKIPHTDGNF
HPSPLMGGFGLKHPPPQILIKNTPVPADPPTTFNQAKLASFITQYSTGQVSVEIEWELQKENSK
RWNPEIQYTSNYYKSTNVDFAVNTEGTYSEPRPIGTRYLTRNL
Spark100_X21:
pSpark100-163-2x-cmycNLS (SEQ ID NO: 79)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKPAAKRVKLDEDPAAKRVKLDKGQQPAKKRL
NFGQTGDSESVPDPQPIGEPPAAPSGVGPNTMAAGGGAPMADNNEGADGVGSSSGNWHCDSTWL
GDRVITTSTRTWALPTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSEYQLPYVLGSAHQGC
LPPFPADVFMIPQYGYLTLNNGSQAVGRSSFYCLEYFPSQMLRTGNNFEFSYNFEDVPFHSSYA
HSQSLDRLMNPLIDQYLYYLSRTQSTGGTAGTQQLLFSQAGPNNMSAQAKNWLPGPCYRQQRVS
TTLSQNNNSNFAWTGATKYHLNGRDSLVNPGVAMATHKDDEERFFPSSGVLMFGKQGAGKDNVD
YSSVMLTSEEEIKTTNPVATEQYGVVADNLQQQNAAPIVGAVNSQGALPGMVWQNRDVYLQGPI
WAKIPHTDGNFHPSPLMGGFGLKHPPPQILIKNTPVPADPPTTFNQAKLASFITQYSTGQVSVE
IEWELQKENSKRWNPEIQYTSNYYKSTNVDFAVNTEGTYSEPRPIGTRYLTRNL
Spark100_X22:
pSpark100-163-SV40 NLS (SEQ ID NO: 80)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKPKKKRKVKGQQPAKKRLNFGQTGDSESVPD
PQPIGEPPAAPSGVGPNTMAAGGGAPMADNNEGADGVGSSSGNWHCDSTWLGDRVITTSTRTWA
LPTYNNHLYKQISNGTSGGSTNDNTYFGYSTPWGYFDFNRFHCHFSPRDWQRLINNNWGFRPKR
LNFKLFNIQVKEVTQNEGTKTIANNLTSTIQVFTDSEYQLPYVLGSAHQGCLPPFPADVFMIPQ
YGYLTLNNGSQAVGRSSFYCLEYFPSQMLRTGNNFEFSYNFEDVPFHSSYAHSQSLDRLMNPLI
DQYLYYLSRTQSTGGTAGTQQLLFSQAGPNNMSAQAKNWLPGPCYRQQRVSTILSQNNNSNFAW
TGATKYHLNGRDSLVNPGVAMATHKDDEERFFPSSGVLMFGKQGAGKDNVDYSSVMLTSEEEIK
TTNPVATEQYGVVADNLQQQNAAPIVGAVNSQGALPGMVWQNRDVYLQGPIWAKIPHTDGNFHP
SPLMGGFGLKHPPPQILIKNTPVPADPPTTFNQAKLASFITQYSTGQVSVEIEWELQKENSKRW
NPEIQYTSNYYKSTNVDFAVNTEGTYSEPRPIGTRYLTRNL
Spark100_X23
pSpark100-163-2x-SV40 (SEQ ID NO: 81)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKPKKKRKVEDPKKKRKVKGQQPAKKRLNFGQ
TGDSESVPDPQPIGEPPAAPSGVGPNTMAAGGGAPMADNNEGADGVGSSSGNWHCDSTWLGDRV
ITTSTRTWALPTYNNHLYKQISNGTSGGSTNDNTYFGYSTPWGYFDFNRFHCHFSPRDWQRLIN
NNWGFRPKRLNFKLFNIQVKEVTQNEGTKTIANNLTSTIQVFTDSEYQLPYVLGSAHQGCLPPF
PADVFMIPQYGYLTLNNGSQAVGRSSFYCLEYFPSQMLRTGNNFEFSYNFEDVPFHSSYAHSQS
LDRLMNPLIDQYLYYLSRTQSTGGTAGTQQLLFSQAGPNNMSAQAKNWLPGPCYRQQRVSTTLS
QNNNSNFAWTGATKYHLNGRDSLVNPGVAMATHKDDEERFFPSSGVLMFGKQGAGKDNVDYSSV
MLTSEEEIKTTNPVATEQYGVVADNLQQQNAAPIVGAVNSQGALPGMVWQNRDVYLQGPIWAKI
PHTDGNFHPSPLMGGFGLKHPPPQILIKNTPVPADPPTTFNQAKLASFITQYSTGQVSVEIEWE
LQKENSKRWNPEIQYTSNYYKSTNVDFAVNTEGTYSEPRPIGTRYLTRNL
Spark100_X24:
pSpark100-163-class3NLS (SEQ ID NO: 82)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKAAAKRSWSAAFKGQQPAKKRLNFGQTGDSE
SVPDPQPIGEPPAAPSGVGPNTMAAGGGAPMADNNEGADGVGSSSGNWHCDSTWLGDRVITTST
RTWALPTYNNHLYKQISNGTSGGSTNDNTYFGYSTPWGYFDFNRFHCHFSPRDWQRLINNNWGF
RPKRLNFKLFNIQVKEVTQNEGTKTIANNLTSTIQVFTDSEYQLPYVLGSAHQGCLPPFPADVF
MIPQYGYLTLNNGSQAVGRSSFYCLEYFPSQMLRTGNNFEFSYNFEDVPFHSSYAHSQSLDRLM
NPLIDQYLYYLSRTQSTGGTAGTQQLLFSQAGPNNMSAQAKNWLPGPCYRQQRVSTTLSQNNNS
NFAWTGATKYHLNGRDSLVNPGVAMATHKDDEERFFPSSGVLMFGKQGAGKDNVDYSSVMLTSE
EEIKTTNPVATEQYGVVADNLQQQNAAPIVGAVNSQGALPGMVWQNRDVYLQGPIWAKIPHTDG
NFHPSPLMGGFGLKHPPPQILIKNTPVPADPPTTFNQAKLASFITQYSTGQVSVEIEWELQKEN
SKRWNPEIQYTSNYYKSTNVDFAVNTEGTYSEPRPIGTRYLTRNL
Spark100_X25:
pSpark100-163-2x-c1ass3NLS (SEQ ID NO: 83)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKAAAKRSWSAAFEDAAAKRSWSAAFKGQQPA
KKRLNFGQTGDSESVPDPQPIGEPPAAPSGVGPNTMAAGGGAPMADNNEGADGVGSSSGNWHCD
STWLGDRVITTSTRTWALPTYNNHLYKQISNGTSGGSTNDNTYFGYSTPWGYFDFNRFHCHFSP
RDWQRLINNNWGFRPKRLNFKLFNIQVKEVTQNEGTKTIANNLTSTIQVFTDSEYQLPYVLGSA
HQGCLPPFPADVFMIPQYGYLTLNNGSQAVGRSSFYCLEYFPSQMLRTGNNFEFSYNFEDVPFH
SSYAHSQSLDRLMNPLIDQYLYYLSRTQSTGGTAGTQQLLFSQAGPNNMSAQAKNWLPGPCYRQ
QRVSTTLSQNNNSNFAWTGATKYHLNGRDSLVNPGVAMATHKDDEERFFPSSGVLMFGKQGAGK
DNVDYSSVMLTSEEEIKTTNPVATEQYGVVADNLQQQNAAPIVGAVNSQGALPGMVWQNRDVYL
QGPIWAKIPHTDGNFHPSPLMGGFGLKHPPPQILIKNTPVPADPPTTFNQAKLASFITQYSTGQ
VSVEIEWELQKENSKRWNPEIQYTSNYYKSTNVDFAVNTEGTYSEPRPIGTRYLTRNL
Spark100-X42:
Spark100-33-cmycNLS (SEQ ID NO: 105)
MAADGYLPDWLEDNLSEGIREWWDLKPGAPKPPAAKRVKLDKANQQKQDNGRGLVLPGYKYLGP
FNGLDKGEPVNAADAAALEHDKAYDQQLQAGDNPYLRYNHADAEFQERLQEDTSFGGNLGRAVF
QAKKRVLEPLGLVESPVKTAPGKKRPVEPSPQRSPDSSTGIGKKGQQPAKKRLNFGQTGDSESV
PDPQPIGEPPAAPSGVGPNTMAAGGGAPMADNNEGADGVGSSSGNWHCDSTWLGDRVITTSTRT
WALPTYNNHLYKQISNGTSGGSTNDNTYFGYSTPWGYFDFNRFHCHFSPRDWQRLINNNWGFRP
KRLNFKLFNIQVKEVTQNEGTKTIANNLTSTIQVFTDSEYQLPYVLGSAHQGCLPPFPADVFMI
PQYGYLTLNNGSQAVGRSSFYCLEYFPSQMLRTGNNFEFSYNFEDVPFHSSYAHSQSLDRLMNP
LIDQYLYYLSRTQSTGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKDNVDYSSVMLTSEEE
IKTTNPVATEQYGVVADNLQQQNAAPIVGAVNSQGALPGMVWQNRDVYLQGPIWAKIPHTDGNF
HPSPLMGGFGLKHPPPQILIKNTPVPADPPTTFNQAKLASFITQYSTGQVSVEIEWELQKENSK
RWNPEIQYTSNYYKSTNVDFAVNTEGTYSEPRPIGTRYLTRNL
Spark100-X43:
Spark100-35-cmycNLS (SEQ ID NO: 106)
MAADGYLPDWLEDNLSEGIREWWDLKPGAPKPKAPAAKRVKLDNQQKQDNGRGLVLPGYKYLGP
FNGLDKGEPVNAADAAALEHDKAYDQQLQAGDNPYLRYNHADAEFQERLQEDTSFGGNLGRAVF
QAKKRVLEPLGLVESPVKTAPGKKRPVEPSPQRSPDSSTGIGKKGQQPAKKRLNFGQTGDSESV
PDPQPIGEPPAAPSGVGPNTMAAGGGAPMADNNEGADGVGSSSGNWHCDSTWLGDRVITTSTRT
WALPTYNNHLYKQISNGTSGGSTNDNTYFGYSTPWGYFDFNRFHCHFSPRDWQRLINNNWGFRP
KRLNFKLFNIQVKEVTQNEGTKTIANNLTSTIQVFTDSEYQLPYVLGSAHQGCLPPFPADVFMI
PQYGYLTLNNGSQAVGRSSFYCLEYFPSQMLRTGNNFEFSYNFEDVPFHSSYAHSQSLDRLMNP
LIDQYLYYLSRTQSTGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKDNVDYSSVMLTSEEE
IKTTNPVATEQYGVVADNLQQQNAAPIVGAVNSQGALPGMVWQNRDVYLQGPIWAKIPHTDGNF
HPSPLMGGFGLKHPPPQILIKNTPVPADPPTTFNQAKLASFITQYSTGQVSVEIEWELQKENSK
RWNPEIQYTSNYYKSTNVDFAVNTEGTYSEPRPIGTRYLTRNL
Spark100-X44:
Spark100-33-2xcmycNLS (SEQ ID NO: 107)
MAADGYLPDWLEDNLSEGIREWWDLKPGAPKPPAAKRVKLDEDPAAKRVKLDKANQQKQDNGRG
LVLPGYKYLGPFNGLDKGEPVNAADAAALEHDKAYDQQLQAGDNPYLRYNHADAEFQERLQEDT
SFGGNLGRAVFQAKKRVLEPLGLVESPVKTAPGKKRPVEPSPQRSPDSSTGIGKKGQQPAKKRL
NFGQTGDSESVPDPQPIGEPPAAPSGVGPNTMAAGGGAPMADNNEGADGVGSSSGNWHCDSTWL
GDRVITTSTRTWALPTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSEYQLPYVLGSAHQGC
LPPFPADVFMIPQYGYLTLNNGSQAVGRSSFYCLEYFPSQMLRTGNNFEFSYNFEDVPFHSSYA
HSQSLDRLMNPLIDQYLYYLSRTQSTGGTAGTQQLLFSQAGPNNMSAQAKNWLPGPCYRQQRVS
TTLSQNNNSNFAWTGATKYHLNGRDSLVNPGVAMATHKDDEERFFPSSGVLMFGKQGAGKDNVD
YSSVMLTSEEEIKTTNPVATEQYGVVADNLQQQNAAPIVGAVNSQGALPGMVWQNRDVYLQGPI
WAKIPHTDGNFHPSPLMGGFGLKHPPPQILIKNTPVPADPPTTFNQAKLASFITQYSTGQVSVE
IEWELQKENSKRWNPEIQYTSNYYKSTNVDFAVNTEGTYSEPRPIGTRYLTRNL
Spark100-X45:
Spark100-35-2xcmycNLS (SEQ ID NO: 108)
MAADGYLPDWLEDNLSEGIREWWDLKPGAPKPKAPAAKRVKLDEDPAAKRVKLDNQQKQDNGRG
LVLPGYKYLGPFNGLDKGEPVNAADAAALEHDKAYDQQLQAGDNPYLRYNHADAEFQERLQEDT
SFGGNLGRAVFQAKKRVLEPLGLVESPVKTAPGKKRPVEPSPQRSPDSSTGIGKKGQQPAKKRL
NFGQTGDSESVPDPQPIGEPPAAPSGVGPNTMAAGGGAPMADNNEGADGVGSSSGNWHCDSTWL
GDRVITTSTRTWALPTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSEYQLPYVLGSAHQGC
LPPFPADVFMIPQYGYLTLNNGSQAVGRSSFYCLEYFPSQMLRTGNNFEFSYNFEDVPFHSSYA
HSQSLDRLMNPLIDQYLYYLSRTQSTGGTAGTQQLLFSQAGPNNMSAQAKNWLPGPCYRQQRVS
TTLSQNNNSNFAWTGATKYHLNGRDSLVNPGVAMATHKDDEERFFPSSGVLMFGKQGAGKDNVD
YSSVMLTSEEEIKTTNPVATEQYGVVADNLQQQNAAPIVGAVNSQGALPGMVWQNRDVYLQGPI
WAKIPHTDGNFHPSPLMGGFGLKHPPPQILIKNTPVPADPPTTFNQAKLASFITQYSTGQVSVE
IEWELQKENSKRWNPEIQYTSNYYKSTNVDFAVNTEGTYSEPRPIGTRYLTRNL
Spark100-X46:
Spark100-35-3xcmycNLS (SEQ ID NO: 109)
MAADGYLPDWLEDNLSEGIREWWDLKPGAPKPKAPAAKRVKLDEDPAAKRVKLDEDPAAKRVKL
DNQQKQDNGRGLVLPGYKYLGPFNGLDKGEPVNAADAAALEHDKAYDQQLQAGDNPYLRYNHAD
AEFQERLQEDTSFGGNLGRAVFQAKKRVLEPLGLVESPVKTAPGKKRPVEPSPQRSPDSSTGIG
KKGQQPAKKRLNFGQTGDSESVPDPQPIGEPPAAPSGVGPNTMAAGGGAPMADNNEGADGVGSS
SGNWHCDSTWLGDRVITTSTRTWALPTYNNHLYKQISNGTSGGSTNDNTYFGYSTPWGYFDFNR
FHCHFSPRDWQRLINNNWGFRPKRLNFKLFNIQVKEVTQNEGTKTIANNLTSTIQVFTDSEYQL
PYVLGSAHQGCLPPFPADVFMIPQYGYLTLNNGSQAVGRSSFYCLEYFPSQMLRTGNNFEFSYN
FEDVPFHSSYAHSQSLDRLMNPLIDQYLYYLSRTQSTGGTAGTQQLLFSQAGPNNMSAQAKNWL
PGPCYRQQRVSTTLSQNNNSNFAWTGATKYHLNGRDSLVNPGVAMATHKDDEERFFPSSGVLMF
GKQGAGKDNVDYSSVMLTSEEEIKTTNPVATEQYGVVADNLQQQNAAPIVGAVNSQGALPGMVW
QNRDVYLQGPIWAKIPHTDGNFHPSPLMGGFGLKHPPPQILIKNTPVPADPPTTFNQAKLASFI
TQYSTGQVSVEIEWELQKENSKRWNPEIQYTSNYYKSTNVDFAVNTEGTYSEPRPIGTRYLTRN
L
Spark100-X47:
Spark100-35-1xClass3NLS (SEQ ID NO: 110)
MAADGYLPDWLEDNLSEGIREWWDLKPGAPKPKAAAAKRSWSAAFNQQKQDNGRGLVLPGYKYL
GPFNGLDKGEPVNAADAAALEHDKAYDQQLQAGDNPYLRYNHADAEFQERLQEDTSFGGNLGRA
VFQAKKRVLEPLGLVESPVKTAPGKKRPVEPSPQRSPDSSTGIGKKGQQPAKKRLNFGQTGDSE
SVPDPQPIGEPPAAPSGVGPNTMAAGGGAPMADNNEGADGVGSSSGNWHCDSTWLGDRVITTST
RTWALPTYNNHLYKQISNGTSGGSTNDNTYFGYSTPWGYFDFNRFHCHFSPRDWQRLINNNWGF
RPKRLNFKLFNIQVKEVTQNEGTKTIANNLTSTIQVFTDSEYQLPYVLGSAHQGCLPPFPADVF
MIPQYGYLTLNNGSQAVGRSSFYCLEYFPSQMLRTGNNFEFSYNFEDVPFHSSYAHSQSLDRLM
NPLIDQYLYYLSRTQSTGGTAGTQQLLFSQAGPNNMSAQAKNWLPGPCYRQQRVSTTLSQNNNS
NFAWTGATKYHLNGRDSLVNPGVAMATHKDDEERFFPSSGVLMFGKQGAGKDNVDYSSVMLTSE
EEIKTTNPVATEQYGVVADNLQQQNAAPIVGAVNSQGALPGMVWQNRDVYLQGPIWAKIPHTDG
NFHPSPLMGGFGLKHPPPQILIKNTPVPADPPTTFNQAKLASFITQYSTGQVSVEIEWELQKEN
SKRWNPEIQYTSNYYKSTNVDFAVNTEGTYSEPRPIGTRYLTRNL
Spark100-X48:
Spark100-35-2xC1ass3NLS (SEQ ID NO: 111)
MAADGYLPDWLEDNLSEGIREWWDLKPGAPKPKAAAAKRSWSAAFEDAAAKRSWSAAFNQQKQD
NGRGLVLPGYKYLGPFNGLDKGEPVNAADAAALEHDKAYDQQLQAGDNPYLRYNHADAEFQERL
QEDTSFGGNLGRAVFQAKKRVLEPLGLVESPVKTAPGKKRPVEPSPQRSPDSSTGIGKKGQQPA
KKRLNFGQTGDSESVPDPQPIGEPPAAPSGVGPNTMAAGGGAPMADNNEGADGVGSSSGNWHCD
STWLGDRVITTSTRTWALPTYNNHLYKQISNGTSGGSTNDNTYFGYSTPWGYFDFNRFHCHFSP
RDWQRLINNNWGFRPKRLNFKLFNIQVKEVTQNEGTKTIANNLTSTIQVFTDSEYQLPYVLGSA
HQGCLPPFPADVFMIPQYGYLTLNNGSQAVGRSSFYCLEYFPSQMLRTGNNFEFSYNFEDVPFH
SSYAHSQSLDRLMNPLIDQYLYYLSRTQSTGGTAGTQQLLFSQAGPNNMSAQAKNWLPGPCYRQ
QRVSTTLSQNNNSNFAWTGATKYHLNGRDSLVNPGVAMATHKDDEERFFPSSGVLMFGKQGAGK
DNVDYSSVMLTSEEEIKTTNPVATEQYGVVADNLQQQNAAPIVGAVNSQGALPGMVWQNRDVYL
QGPIWAKIPHTDGNFHPSPLMGGFGLKHPPPQILIKNTPVPADPPTTFNQAKLASFITQYSTGQ
VSVEIEWELQKENSKRWNPEIQYTSNYYKSTNVDFAVNTEGTYSEPRPIGTRYLTRNL
Spark100-X49:
Spark100-35-3xC1ass3NLS (SEQ ID NO: 112)
MAADGYLPDWLEDNLSEGIREWWDLKPGAPKPKAAAAKRSWSAAFEDAAAKASWSAAFEDAAAK
RSWSAAFNQQKQDNGRGLVLPGYKYLGPFNGLDKGEPVNAADAAALEHDKAYDQQLQAGDNPYL
RYNHADAEFQERLQEDTSFGGNLGRAVFQAKKRVLEPLGLVESPVKTAPGKKRPVEPSPQRSPD
SSTGIGKKGQQPAKKRLNFGQTGDSESVPDPQPIGEPPAAPSGVGPNTMAAGGGAPMADNNEGA
DGVGSSSGNWHCDSTWLGDRVITTSTRTWALPTYNNHLYKQISNGTSGGSTNDNTYFGYSTPWG
YFDFNRFHCHFSPRDWQRLINNNWGFRPKRLNFKLFNIQVKEVTQNEGTKTIANNLTSTIQVFT
DSEYQLPYVLGSAHQGCLPPFPADVFMIPQYGYLTLNNGSQAVGRSSFYCLEYFPSQMLRTGNN
FEFSYNFEDVPFHSSYAHSQSLDRLMNPLIDQYLYYLSRTQSTGGTAGTQQLLFSQAGPNNMSA
QAKNWLPGPCYRQQRVSTTLSQNNNSNFAWTGATKYHLNGRDSLVNPGVAMATHKDDEERFFPS
SGVLMFGKQGAGKDNVDYSSVMLTSEEEIKTTNPVATEQYGVVADNLQQQNAAPIVGAVNSQGA
LPGMVWQNRDVYLQGPIWAKIPHTDGNFHPSPLMGGFGLKHPPPQILIKNTPVPADPPTTFNQA
KLASFITQYSTGQVSVEIEWELQKENSKRWNPEIQYTSNYYKSTNVDFAVNTEGTYSEPRPIGT
RYLTRNL
Spark100-X50:
Spark100-35-hnRNP_NLS (SEQ ID NO: 113)
MAADGYLPDWLEDNLSEGIREWWDLKPGAPKPKAYGDYSNQQSGYGKVSRRGGHQNSYKPYNQQ
KQDNGRGLVLPGYKYLGPFNGLDKGEPVNAADAAALEHDKAYDQQLQAGDNPYLRYNHADAEFQ
ERLQEDTSFGGNLGRAVFQAKKRVLEPLGLVESPVKTAPGKKRPVEPSPQRSPDSSTGIGKKGQ
QPAKKRLNFGQTGDSESVPDPQPIGEPPAAPSGVGPNTMAAGGGAPMADNNEGADGVGSSSGNW
HCDSTWLGDRVITTSTRTWALPTYNNHLYKQISNGTSGGSTNDNTYFGYSTPWGYFDFNRFHCH
FSPRDWQRLINNNWGFRPKRLNFKLFNIQVKEVTQNEGTKTIANNLTSTIQVFTDSEYQLPYVL
GSAHQGCLPPFPADVFMIPQYGYLTLNNGSQAVGRSSFYCLEYFPSQMLRTGNNFEFSYNFEDV
PFHSSYAHSQSLDRLMNPLIDQYLYYLSRTQSTGGTAGTQQLLFSQAGPNNMSAQAKNWLPGPC
YRQQRVSTTLSQNNNSNFAWTGATKYHLNGRDSLVNPGVAMATHKDDEERFFPSSGVLMFGKQG
AGKDNVDYSSVMLTSEEEIKTTNPVATEQYGVVADNLQQQNAAPIVGAVNSQGALPGMVWQNRD
VYLQGPIWAKIPHTDGNFHPSPLMGGFGLKHPPPQILIKNTPVPADPPTTFNQAKLASFITQYS
TGQVSVEIEWELQKENSKRWNPEIQYTSNYYKSTNVDFAVNTEGTYSEPRPIGTRYLTRNL
Spark100-X51:
Spark100-35-1X-SV40 NLS (SEQ ID NO: 114)
MAADGYLPDWLEDNLSEGIREWWDLKPGAPKPKAPKKKRKVNQQKQDNGRGLVLPGYKYLGPFN
GLDKGEPVNAADAAALEHDKAYDQQLQAGDNPYLRYNHADAEFQERLQEDTSFGGNLGRAVFQA
KKRVLEPLGLVESPVKTAPGKKRPVEPSPQRSPDSSTGIGKKGQQPAKKRLNFGQTGDSESVPD
PQPIGEPPAAPSGVGPNTMAAGGGAPMADNNEGADGVGSSSGNWHCDSTWLGDRVITTSTRTWA
LPTYNNHLYKQISNGTSGGSTNDNTYFGYSTPWGYFDFNRFHCHFSPRDWQRLINNNWGFRPKR
LNFKLFNIQVKEVTQNEGTKTIANNLTSTIQVFTDSEYQLPYVLGSAHQGCLPPFPADVFMIPQ
YGYLTLNNGSQAVGRSSFYCLEYFPSQMLRTGNNFEFSYNFEDVPFHSSYAHSQSLDRLMNPLI
DQYLYYLSRTQSTGGTAGTQQLLFSQAGPNNMSAQAKNWLPGPCYRQQRVSTILSQNNNSNFAW
TGATKYHLNGRDSLVNPGVAMATHKDDEERFFPSSGVLMFGKQGAGKDNVDYSSVMLTSEEEIK
TTNPVATEQYGVVADNLQQQNAAPIVGAVNSQGALPGMVWQNRDVYLQGPIWAKIPHTDGNFHP
SPLMGGFGLKHPPPQILIKNTPVPADPPTTFNQAKLASFITQYSTGQVSVEIEWELQKENSKRW
NPEIQYTSNYYKSTNVDFAVNTEGTYSEPRPIGTRYLTRNL
Spark100-X52: K→Y R mutation
Spark100_K333R (SEQ ID NO: 115)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKKGQQPAKKRLNFGQTGDSESVPDPQPIGEP
PAAPSGVGPNTMAAGGGAPMADNNEGADGVGSSSGNWHCDSTWLGDRVITTSTRTWALPTYNNH
LYKQISNGTSGGSTNDNTYFGYSTPWGYFDFNRFHCHFSPRDWQRLINNNWGFRPKRLNFKLFN
IQVKEVTQNEGTRTIANNLTSTIQVFTDSEYQLPYVLGSAHQGCLPPFPADVFMIPQYGYLTLN
NGSQAVGRSSFYCLEYFPSQMLRTGNNFEFSYNFEDVPFHSSYAHSQSLDRLMNPLIDQYLYYL
SRTQSTGGTAGTQQLLFSQAGPNNMSAQAKNWLPGPCYRQQRVSTILSQNNNSNFAWTGATKYH
LNGRDSLVNPGVAMATHKDDEERFFPSSGVLMFGKQGAGKDNVDYSSVMLTSEEEIKTTNPVAT
EQYGVVADNLQQQNAAPIVGAVNSQGALPGMVWQNRDVYLQGPIWAKIPHTDGNFHPSPLMGGF
GLKHPPPQILIKNTPVPADPPTTFNQAKLASFITQYSTGQVSVEIEWELQKENSKRWNPEIQYT
SNYYKSTNVDFAVNTEGTYSEPRPIGTRYLTRNL
Spark100-X53: K→R mutation
Spark100_K530R (SEQ ID NO: 116)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKKGQQPAKKRLNFGQTGDSESVPDPQPIGEP
PAAPSGVGPNTMAAGGGAPMADNNEGADGVGSSSGNWHCDSTWLGDRVITTSTRTWALPTYNNH
LYKQISNGTSGGSTNDNTYFGYSTPWGYFDFNRFHCHFSPRDWQRLINNNWGFRPKRLNFKLFN
IQVKEVTQNEGTKTIANNLTSTIQVFTDSEYQLPYVLGSAHQGCLPPFPADVFMIPQYGYLTLN
NGSQAVGRSSFYCLEYFPSQMLRTGNNFEFSYNFEDVPFHSSYAHSQSLDRLMNPLIDQYLYYL
SRTQSTGGTAGTQQLLFSQAGPNNMSAQAKNWLPGPCYRQQRVSTILSQNNNSNFAWTGATKYH
LNGRDSLVNPGVAMATHRDDEERFFPSSGVLMFGKQGAGKDNVDYSSVMLTSEEEIKTTNPVAT
EQYGVVADNLQQQNAAPIVGAVNSQGALPGMVWQNRDVYLQGPIWAKIPHTDGNFHPSPLMGGF
GLKHPPPQILIKNTPVPADPPTTFNQAKLASFITQYSTGQVSVEIEWELQKENSKRWNPEIQYT
SNYYKSTNVDFAVNTEGTYSEPRPIGTRYLTRNL
Spark100-X54:
Spark100_385cmycNLS (SEQ ID NO: 117)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKKGQQPAKKRLNFGQTGDSESVPDPQPIGEP
PAAPSGVGPNTMAAGGGAPMADNNEGADGVGSSSGNWHCDSTWLGDRVITTSTRTWALPTYNNH
LYKQISNGTSGGSTNDNTYFGYSTPWGYFDFNRFHCHFSPRDWQRLINNNWGFRPKRLNFKLFN
IQVKEVTQNEGTKTIANNLTSTIQVFTDSEYQLPYVLGSAHQGCLPPFPADVFMIPQYGYLTLN
PAAKRVKLDNGSQAVGRSSFYCLEYFPSQMLRTGNNFEFSYNFEDVPFHSSYAHSQSLDRLMNP
LIDQYLYYLSRTQSTGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKDNVDYSSVMLTSEEE
IKTTNPVATEQYGVVADNLQQQNAAPIVGAVNSQGALPGMVWQNRDVYLQGPIWAKIPHTDGNF
HPSPLMGGFGLKHPPPQILIKNTPVPADPPTTFNQAKLASFITQYSTGQVSVEIEWELQKENSK
RWNPEIQYTSNYYKSTNVDFAVNTEGTYSEPRPIGTRYLTRNL
Spark100-X55:
pSpark100_451cmycNLS (SEQ ID NO: 118)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKKGQQPAKKRLNFGQTGDSESVPDPQPIGEP
PAAPSGVGPNTMAAGGGAPMADNNEGADGVGSSSGNWHCDSTWLGDRVITTSTRTWALPTYNNH
LYKQISNGTSGGSTNDNTYFGYSTPWGYFDFNRFHCHFSPRDWQRLINNNWGFRPKRLNFKLFN
IQVKEVTQNEGTKTIANNLTSTIQVFTDSEYQLPYVLGSAHQGCLPPFPADVFMIPQYGYLTLN
NGSQAVGRSSFYCLEYFPSQMLRTGNNFEFSYNFEDVPFHSSYAHSQSLDRLMNPLIDQYLYYL
SRPAAKRVKLDTQSTGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKDNVDYSSVMLTSEEE
IKTTNPVATEQYGVVADNLQQQNAAPIVGAVNSQGALPGMVWQNRDVYLQGPIWAKIPHTDGNF
HPSPLMGGFGLKHPPPQILIKNTPVPADPPTTFNQAKLASFITQYSTGQVSVEIEWELQKENSK
RWNPEIQYTSNYYKSTNVDFAVNTEGTYSEPRPIGTRYLTRNL
Spark100-X56:
pSpark100_457cmycNLS (SEQ ID NO: 119)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKKGQQPAKKRLNFGQTGDSESVPDPQPIGEP
PAAPSGVGPNTMAAGGGAPMADNNEGADGVGSSSGNWHCDSTWLGDRVITTSTRTWALPTYNNH
LYKQISNGTSGGSTNDNTYFGYSTPWGYFDFNRFHCHFSPRDWQRLINNNWGFRPKRLNFKLFN
IQVKEVTQNEGTKTIANNLTSTIQVFTDSEYQLPYVLGSAHQGCLPPFPADVFMIPQYGYLTLN
NGSQAVGRSSFYCLEYFPSQMLRTGNNFEFSYNFEDVPFHSSYAHSQSLDRLMNPLIDQYLYYL
SRTQSTGGPAAKRVKLDTAGTQQLLFSQAGPNNMSAQAKNWLPGPCYRQQRVSTILSQNNNSNF
AWTGATKYHLNGRDSLVNPGVAMATHKDDEERFFPSSGVLMFGKQGAGKDNVDYSSVMLTSEEE
IKTTNPVATEQYGVVADNLQQQNAAPIVGAVNSQGALPGMVWQNRDVYLQGPIWAKIPHTDGNF
HPSPLMGGFGLKHPPPQILIKNTPVPADPPTTFNQAKLASFITQYSTGQVSVEIEWELQKENSK
RWNPEIQYTSNYYKSTNVDFAVNTEGTYSEPRPIGTRYLTRNL
Spark100-X57:
pSpark100-589_cmycNLS (SEQ ID NO: 120)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKKGQQPAKKRLNFGQTGDSESVPDPQPIGEP
PAAPSGVGPNTMAAGGGAPMADNNEGADGVGSSSGNWHCDSTWLGDRVITTSTRTWALPTYNNH
LYKQISNGTSGGSTNDNTYFGYSTPWGYFDFNRFHCHFSPRDWQRLINNNWGFRPKRLNFKLFN
IQVKEVTQNEGTKTIANNLTSTIQVFTDSEYQLPYVLGSAHQGCLPPFPADVFMIPQYGYLTLN
NGSQAVGRSSFYCLEYFPSQMLRTGNNFEFSYNFEDVPFHSSYAHSQSLDRLMNPLIDQYLYYL
SRTQSTGGTAGTQQLLFSQAGPNNMSAQAKNWLPGPCYRQQRVSTTLSQNNNSNFAWTGATKYH
LNGRDSLVNPGVAMATHKDDEERFFPSSGVLMFGKQGAGKDNVDYSSVMLTSEEEIKTTNPVAT
EQYGVVADNLQQPAAKRVKLDQNAAPIVGAVNSQGALPGMVWQNRDVYLQGPIWAKIPHTDGNF
HPSPLMGGFGLKHPPPQILIKNTPVPADPPTTFNQAKLASFITQYSTGQVSVEIEWELQKENSK
RWNPEIQYTSNYYKSTNVDFAVNTEGTYSEPRPIGTRYLTRNL
Spark100-X58:
pSpark100-591_cmycNLS (SEQ ID NO: 121)
MAADGYLPDWLEDNLSEGIREWWDLKPGAPKPKANQQKQDNGRGLVLPGYKYLGPFNGLDKGEP
VNAADAAALEHDKAYDQQLQAGDNPYLRYNHADAEFQERLQEDTSFGGNLGRAVFQAKKRVLEP
LGLVESPVKTAPGKKRPVEPSPQRSPDSSTGIGKKGQQPAKKRLNFGQTGDSESVPDPQPIGEP
PAAPSGVGPNTMAAGGGAPMADNNEGADGVGSSSGNWHCDSTWLGDRVITTSTRTWALPTYNNH
LYKQISNGTSGGSTNDNTYFGYSTPWGYFDFNRFHCHFSPRDWQRLINNNWGFRPKRLNFKLFN
IQVKEVTQNEGTKTIANNLTSTIQVFTDSEYQLPYVLGSAHQGCLPPFPADVFMIPQYGYLTLN
NGSQAVGRSSFYCLEYFPSQMLRTGNNFEFSYNFEDVPFHSSYAHSQSLDRLMNPLIDQYLYYL
SRTQSTGGTAGTQQLLFSQAGPNNMSAQAKNWLPGPCYRQQRVSTILSQNNNSNFAWTGATKYH
LNGRDSLVNPGVAMATHKDDEERFFPSSGVLMFGKQGAGKDNVDYSSVMLTSEEEIKTTNPVAT
EQYGVVADNLQQQNPAAKRVKLDAAPIVGAVNSQGALPGMVWQNRDVYLQGPIWAKIPHTDGNF
HPSPLMGGFGLKHPPPQILIKNTPVPADPPTTFNQAKLASFITQYSTGQVSVEIEWELQKENSK
RWNPEIQYTSNYYKSTNVDFAVNTEGTYSEPRPIGTRYLTRNL

### Example 8

AAV2 VP1
(SEQ ID NO: 122)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL
FVIII-BDD, showing the SQ sequence underlined
(SEQ ID NO: 123)
MQIELSTCFFLCLLRFCFSATRRYYLGAVELSWDYMQSDLGELPVDARFP
PRVPKSFPFNTSVVYKKTLFVEFTDHLFNIAKPRPPWMGLLGPTIQAEVY
DTVVITLKNMASHPVSLHAVGVSYWKASEGAEYDDQTSQREKEDDKVFPG
GSHTYVWQVLKENGPMASDPLCLTYSYLSHVDLVKDLNSGLIGALLVCRE
GSLAKEKTQTLHKFILLFAVFDEGKSWHSETKNSLMQDRDAASARAWPKM
HTVNGYVNRSLPGLIGCHRKSVYWHVIGMGTTPEVHSIFLEGHTFLVRNH
RQASLEISPITFLTAQTLLMDLGQFLLFCHISSHQHDGMEAYVKVDSCPE
EPQLRMKNNEEAEDYDDDLTDSEMDVVRFDDDNSPSFIQIRSVAKKHPKT
WVHYIAAEEEDWDYAPLVLAPDDRSYKSQYLNNGPQRIGRKYKKVRFMAY
TDETFKTREAIQHESGILGPLLYGEVGDTLLIIFKNQASRPYNIYPHGIT
DVRPLYSRRLPKGVKHLKDFPILPGEIFKYKWTVTVEDGPTKSDPRCLTR
YYSSFVNMERDLASGLIGPLLICYKESVDQRGNQIMSDKRNVILFSVFDE
NRSWYLTENIQRFLPNPAGVQLEDPEFQASNIMHSINGYVFDSLQLSVCL
HEVAYWYILSIGAQTDFLSVFFSGYTFKHKMVYEDTLTLFPFSGETVFMS
MENPGLWILGCHNSDFRNRGMTALLKVSSCDKNTGDYYEDSYEDISAYLL
SKNNAIEPRSFSQNPPVLKRHQREITRTTLQSDQEEIDYDDTISVEMKKE
DFDIYDEDENQSPRSFQKKTRHYFIAAVERLWDYGMSSSPHVLRNRAQSG
SVPQFKKVVFQEFTDGSFTQPLYRGELNEHLGLLGPYIRAEVEDNIMVTF
RNQASRPYSFYSSLISYEEDQRQGAEPRKNFVKPNETKTYFWKVQHHMAP
TKDEFDCKAWAYFSDVDLEKDVHSGLIGPLLVCHTNTLNPAHGRQVTVQE
FALFFTIFDETKSWYFTENMERNCRAPCNIQMEDPTFKENYRFHAINGYI
MDTLPGLVMAQDQRIRWYLLSMGSNENIHSIHFSGHVFTVRKKEEYKMAL
YNLYPGVFETVEMLPSKAGIWRVECLIGEHLHAGMSTLFLVYSNKCQTPL
GMASGHIRDFQITASGQYGQWAPKLARLHYSGSINAWSTKEPFSWIKVDL
LAPMIIHGIKTQGARQKFSSLYISQFIIMYSLDGKKWQTYRGNSTGTLMV
FFGNVDSSGIKHNIFNPPIIARYIRLHPTHYSIRSTLRMELMGCDLNSCS
MPLGMESKAISDAQITASSYFTNMFATWSPSKARLHLQGRSNAWRPQVNN
PKEWLQVDFQKTMKVTGVTTQGVKSLLTSMYVKEFLISSSQDGHQWTLFF
QNGKVKVFQGNQDSFTPVVNSLDPPLLTRYLRIHPQSWVHQIALRMEVLG
CEAQDLY
SQ sequence
(SEQ ID NO: 124)
SFSQNPPVLKRHQR
Transthyretin (TTR) promoter
(SEQ ID NO: 125)
GTCTGTCTGCACATTTCGTAGAGCGAGTGTTCCGATACTCTAATCTCCCT
AGGCAAGGTTCATATTTGTGTAGGTTACTTATTCTCCTTTTGTTGACTAA
GTCAATAATCAGAATCAGCAGGTTTGGAGTCAGCTTGGCAGGGATCAGCA
GCCTGGGTTGGAAGGAGGGGGTATAAAAGCCCCTTCACCAGGAGAAGCCG
TCACACAGATCCACAAGCTCCT
Mutant transthyretin (TTRmut) promoter
(SEQ ID NO: 126)
GTCTGTCTGCACATTTCGTAGAGCGAGTGTTCCGATACTCTAATCTCCCT
AGGCAAGGTTCATATTGACTTAGGTTACTTATTCTCCTTTTGTTGACTAA
GTCAATAATCAGAATCAGCAGGTTTGGAGTCAGCTTGGCAGGGATCAGCA
GCCTGGGTTGGAAGGAGGGGGTATAAAAGCCCCTTCACCAGGAGAAGCCG

What is claimed:

1. An adeno-associated virus (AAV) capsid protein, wherein said capsid protein is modified from a parental AAV capsid protein to have a peptide insertion comprising a nuclear localization signal (NLS) sequence.

2. An adeno-associated virus (AAV) capsid protein, wherein said capsid protein is modified from a parental capsid protein to have an amino acid substitution at an RXXL site or a (L/P)PXY site, where X can be any amino acid.

3. An adeno-associated virus (AAV) capsid protein, wherein said capsid protein is modified from a parental AAV capsid protein, and wherein said capsid protein is at least 90% identical to SEQ ID NO:1 and has an alanine at amino acid position 517 of SEQ ID NO:1, or is at least 90% identical to SEQ ID NO:59 and has an alanine at position 519 of SEQ ID NO:59, or is at least 90% identical to a capsid protein of another AAV serotype and has an alanine at the corresponding amino acid position in the capsid protein of the other AAV serotype.

4. An adeno-associated virus (AAV) capsid protein, wherein said capsid protein is modified from a parental AAV capsid protein, and wherein said capsid protein is at least 90% identical to SEQ ID NO:1 and has an arginine at any of amino acid positions 137, 528, 533 and 545 of SEQ ID NO:1, or is at least 90% identical to SEQ ID NO:59 and has an arginine at any of amino acid positions 137, 333 and 530 of SEQ ID NO:59, or is at least 90% identical to a capsid protein of another AAV serotype and has an arginine at any of the corresponding amino acid positions in the capsid protein of the other AAV serotype.

5. The modified AAV capsid protein of any of claims 2-4, wherein said capsid protein further comprises an insertion of a nuclear localization sequence.

6. The modified AAV capsid protein of claim 1 or 5, wherein said nuclear localization sequence does not comprise an AAV nuclear localization sequence.

7. The modified AAV capsid protein of claim 1 or 5, wherein said nuclear localization sequence comprises an additional AAV nuclear localization sequence from the same or different AAV serotype.

8. The modified AAV capsid protein of any of claims 2-4, wherein said capsid protein further comprises an insertion of a cell penetrating peptide.

9. The modified AAV capsid protein of claim 1 or 5-8, wherein said peptide insertion, nuclear localization sequence or cell penetrating peptide has a length of about 5 amino acids to about 60 amino acids.

10. The modified AAV capsid protein of claim 1 or 5-8, wherein said peptide insertion is located at a position within amino acids 1-52 of AAV VP1 capsid protein.

11. The modified AAV capsid protein of claim 1 or 5-8, wherein said peptide insertion is located at a position between basic region 1 (BR1) and basic region 2 (BR2) of said AAV capsid protein.

12. The modified AAV capsid protein of claim 1 or 5-8, wherein said peptide insertion is located at a position between basic region 2 (BR2) and basic region 3 (BR3) of said AAV capsid protein.

13. The modified AAV capsid protein of claim 1 or 5-8, wherein said peptide insertion is in AAV VP1 and/or VP2 and/or VP3 capsid proteins.

14. The modified AAV capsid protein of claim 1 or 5-8, wherein said peptide insertion is not in AAV VP2 and/or VP3 capsid protein.

15. The modified AAV capsid protein of claim 1 or 5-8, wherein said peptide insertion is not in basic region 1 (BR1), basic region 2 (BR2), basic region 3 (BR3), basic region 4 (BR4) or basic region 5 (BR5).

16. The modified AAV capsid protein of claim 1 or 5-8, wherein said peptide insertion is not in a phospholipase A2 (PLA2) domain.

17. The modified AAV capsid protein of claim 1 or 5-8, wherein said peptide insertion is located in loop 3 (aka subloop I in loop IV) of VP1 capsid protein.

18. The modified AAV capsid protein of claim 1 or 5-8, wherein said peptide insertion is located immediately after any amino acid in the range of positions 30-40, 135-141, 147-166, 380-390, 445-460 or 585-595 of VP1 capsid protein.

19. The modified AAV capsid protein of claim 1 or 5-8, wherein said peptide insertion is located immediately after any amino acid in the range of positions 32-33, 34-35, 36-37, 138-139, 139-140, 162-163, 384-385, 450-451, 456-457, 588-589 or 590-591 of VP1 capsid protein.

20. The modified AAV capsid protein of any of claims 1-19, wherein said modified AAV capsid protein has 90% or more identity to a sequence selected from SEQ ID NOs:2-46 and 56-58.

21. The modified AAV capsid protein of any of claims 1-19, wherein said modified AAV capsid protein has 90% or more identity to a sequence selected from SEQ ID NOs:47-55.

22. The modified AAV capsid protein of any of claims 1-19, wherein said modified AAV capsid protein has 90% or more identity to a sequence selected from SEQ ID NOs:60-83.

23. The modified AAV capsid protein of any of claims 1-19, wherein said modified AAV capsid protein has 90% or more identity to a sequence selected from SEQ ID NOs:105-121.

24. The modified AAV capsid protein of any of claims 1 and 5-23, wherein said peptide insertion is 90% or more identical to a sequence selected from SEQ ID NOs:84-104.

25. The modified AAV capsid protein of any of claims 1 and 5-23, wherein said peptide insertion comprises at least two sequences selected from any of SEQ ID NOs:84-92 and 95.

26. The modified AAV capsid protein of claim 25, wherein said at least two sequences selected from any of SEQ ID NOs:84-92 and 95 are the same sequence.

27. The modified AAV capsid protein of claim 26, wherein said at least two sequences selected from any of SEQ ID NOs:84-92 and 95 are different sequences.

28. The modified AAV capsid protein of any of claims 23-27, wherein the at least two sequences are separated by 1-5 intervening amino acid residues.

29. The modified AAV capsid protein of any of claims 1, 5-20 and 22, wherein said peptide insertion comprises a tandem repeat of a sequence selected from any of SEQ ID NOs:84-92 and 95.

30. The modified AAV capsid protein of any of claims 1, 5-20 and 22, wherein said peptide insertion comprises a tandem repeat of at least 2 sequences selected from any of SEQ ID NOs:84-92 and 95 wherein any of the 1^{st} of said at least 2 sequences is positioned at the 5′ end of the 2^{nd} sequence and any of the 2^{nd} of said at least 2 sequences is positioned at the 3′ end of the 1s^{t} sequence.

31. The modified AAV capsid protein of any of claims 1, 5-20 and 22, wherein said peptide insertion comprises a tandem repeat of at least 3 sequences selected from any of SEQ ID NOs:93, 94 and 96-104.

32. The modified AAV capsid protein of any of claims 1, 5-20 and 22, wherein the peptide insertion comprises a tandem repeat of at least 3 sequences selected from any of SEQ ID NOs:84-92 and 95, wherein the 1^{st} of said at least 3 sequences is positioned at the 5′ end of the 2^{nd} sequence, the 2^{nd} of said at least 3 sequences is positioned at the 3′ end of the 1^{st} sequence and the 3^{rd} of said at least 3 sequences is positioned at the 3′ end of the 2^{nd} sequence.

33. The modified AAV capsid protein of any of claims 27-32, wherein the tandem repeats are separated by 1-5 intervening amino acid residues.

34. The modified AAV capsid protein of any of claims 1, 5-20 and 22, wherein said peptide insertion comprises any of SEQ ID NOs:84-104 with one or more amino acid substitutions.

35. The modified AAV capsid protein of any of claims 1, 5-20 and 22, wherein said peptide insertion comprises any of SEQ ID NOs:84-104 with 1-10 amino acid substitutions.

36. The modified AAV capsid protein of any of claims 1, 5-20 and 22, wherein said peptide insertion comprises any of SEQ ID NOs:84-104 with one or more conservative amino acid substitutions.

37. The modified AAV capsid protein of any of claims 1, 5-20 and 22, wherein the AAV capsid protein having the peptide insertion comprises SEQ ID NO:1, SEQ ID NO:59 or SEQ ID NO:122.

38. The modified AAV capsid protein of any of claims 1, 5-20 and 22, wherein the AAV capsid protein having the peptide insertion comprises a VP1, VP2 and/or VP3 capsid sequence having 90% or more identity to Spk200 (SEQ ID NO:1), Spk100 (SEQ ID NO:59), AAV1, AAV2 (SEQ ID NO:122), AAV3, AAV4, AAV5, AAV6, AAV7, AAV8, AAV9, AAV10, AAV11, AAV12, Rh10, or Rh74 VP1, VP2 and/or VP3 sequences.

39. A recombinant adeno-associated virus (rAAV) particle comprising: a) a modified AAV capsid protein of any of claims 1-39; and b) a vector genome comprising a heterologous nucleic acid sequence.

40. A recombinant adeno-associated virus (rAAV) particle comprising a modified AAV capsid protein of any of claims 1-39, wherein said rAAV particle is devoid of a heterologous nucleic acid sequence.

41. The rAAV particle of claim 39 or 40, wherein said peptide insertion does not prevent assembly of the rAAV particle.

42. The rAAV particle of claim 39 or 40, wherein said peptide insertion does not prevent cell infectivity of the rAAV particle.

43. The rAAV particle of claim 39 or 40, wherein said peptide insertion increases or enhances entry or transduction of said rAAV particle into the nucleus of a cell, as compared to entry or transduction into the nucleus of a cell of an rAAV particle comprising said parental AAV capsid protein.

44. The rAAV particle of claim 39 or 40, wherein said peptide insertion increases or enhances escape of said rAAV particle from cell endosomes as compared to escape of an rAAV particle comprising said parental AAV capsid protein from cell endosomes.

45. The rAAV particle of claim 39 or 40, wherein said peptide insertion reduces or decreases degradation of said rAAV particle in lysosomes of cells as compared to degradation in lysosomes of cells of an rAAV particle comprising said parental AAV capsid protein.

46. A plurality of rAAV particles of any of claims 39-45.

47. A pharmaceutical composition comprising the rAAV particle of any of claims 39-45.

48. A pharmaceutical composition comprising the rAAV particles of claim 46.

49. A method for delivering or transferring a heterologous nucleic acid sequence into a mammal or a cell of a mammal, comprising administering the rAAV particle, rAAV particles or pharmaceutical composition of any of claims 39-48 to said mammal or a cell of said mammal.

50. A method of treating a mammal deficient in protein expression or function, comprising administering an effective amount of the rAAV particle or pharmaceutical composition of any of claims 39-48 to said mammal, wherein said heterologous nucleic acid sequence encodes a protein having a function of the deficient protein, and wherein said protein having said function of said deficient protein is expressed in said mammal, thereby treating said mammal deficient in protein expression or function.

51. The rAAV particle or method of any of claim 49 or 50, wherein said heterologous nucleic acid sequence comprises or encodes a blood coagulation Factor.

52. The rAAV particle or method of any of claims 39-51, wherein said heterologous nucleic acid sequence encodes Factor VII, VIII, IX, X, XI, V, XII, II, von Willebrand factor, vitamin K epoxide reductase C1, or gamma-carboxylase.

53. The rAAV particle or method of claim 52, wherein said Factor VIII has a B domain deletion (BDD).

54. The rAAV particle or method of claim 53, wherein said heterologous nucleic acid sequence encoding said factor VIII with the B domain deletion has 20 or fewer, 15 or fewer, or 10 or fewer cytosine-guanine dinucleotides (CpGs).

55. The rAAV particle or method of claim 53, wherein said heterologous nucleic acid sequence encoding saidfactor VIII with the B domain deletion has no more than 5 cytosine-guanine dinucleotides (CpGs).

56. The rAAV particle or method of claim 53, wherein said heterologous nucleic acid sequence encoding said factor VIII with the B domain deletion has 4, 3, 2, 1 or 0 cytosine-guanine dinucleotides (CpGs).

57. The rAAV particle or method of claim 53, wherein said factor VIII comprises SEQ ID NO:123 having a deletion of one or more amino acids of the sequence SFSQNPPVLKRHQR (SEQ ID NO:124), or a deletion of the entire sequence SFSQNPPVLKRHQR.

58. The rAAV particle or method of any of claims 39-50, wherein said heterologous nucleic acid sequence comprises or encodes acid alpha-glucosidase (GAA); ATP7B (copper transporting ATPase2); alpha galactosidase; ASS1 (arginosuccinate synthase); beta-glucocerebrosidase; beta-hexosaminidase A; SERPING1 (C1 protease inhibitor); glucose-6-phosphatase; erythropoietin (EPO; interferon-alpha; interferon-beta; interferon-gamma; an interleukin (IL); any one of interleukins 1-36 (IL-1 through IL-36); interleukin (IL) receptor; a chemokine; chemokine (C—X—C motif) ligand 5 (CXCL5); granulocyte-colony stimulating factor (G-CSF); granulocyte-macrophage colony stimulating factor (GM-CSF); macrophage colony stimulating factor (M-CSF); keratinocyte growth factor (KGF); monocyte chemoattractant protein-1 (MCP-1); tumor necrosis factor (TNF); a tumor necrosis factor (TNF) receptor; alpha-1 antitrypsin; alpha-L-iduronidase; ornithine transcarbamoylase; phenylalanine hydroxylase (PAH); phenylalanine ammonia-lyase (PAL); lipoprotein lipase; an apolipoprotein; low-density lipoprotein receptor (LDL-R); albumin; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein, T-protein, cystic fibrosis transmembrane regulator (CFTR); ATP-binding cassette, sub-family A (ABC1), member 4 (ABCA4); or dystrophin.

59. The rAAV particle or method of any of claims 39-50, wherein said heterologous nucleic acid sequence encodes an inhibitory RNA selected from a short hairpin (sh)RNA, a microRNA (miRNA), a small or short interfering (si)RNA, a trans-splicing RNA, and an antisense RNA.

60. The method of claim 49 or 50, wherein said mammal is human.

61. The method of claim 60, wherein said human has a blood clotting disorder, Pompe disease, Wilson's disease, Fabry disease, citrullinemia type 1, Gaucher disease type 1, Tay Sachs disease, hereditary angioedema (HAE), glycogen storage disease type I (GSDI), anemia, interferon-alpha, interferon-beta, or interferon-gamma related immune disorders, a viral infections, cancer, an inflammatory disease, an immune deficiency, an immune disorder, Crohn's disease, epithelial tissue damage, insulin resistance, emphysema, chronic obstructive pulmonary disease (COPD), mucopolysaccharidosis I (MPS I), ornithine transcarbamylase (OTC) deficiency, phenylketonuria (PKU), lipoprotein lipase deficiency, apolipoprotein (Apo) A-I deficiency, familial hypercholesterolemia (FH), Stargardt disease or hypoalbuminemia.

62. The method of claim 61, wherein said blood clotting disorder is hemophilia A or hemophilia B.

63. The rAAV particle or method of any of claims 39-57, wherein said vector genome further comprises an intron, an expression control element, one or more AAV inverted terminal repeats (ITRs) and/or a filler polynucleotide sequence.

64. The rAAV particle or method of claim 63, wherein said intron is within or flanks said heterologous nucleic acid sequence.

65. The rAAV particle or method of claim 63, wherein said expression control element is operably linked said heterologous nucleic acid sequence.

66. The rAAV particle or method of claim 63, wherein said AAV ITR(s) flanks the 5′ and/or 3′ terminus of said heterologous nucleic acid sequence.

67. The rAAV particle or method of claim 63, wherein said filler polynucleotide sequence flanks the 5′ or 3′terminus of said heterologous nucleic acid sequence.

68. The rAAV particle or method of claim 63, wherein said intron, said expression control element, said one or more AAV, ITRs, and/or said filler polynucleotide sequence has been modified to have reduced cytosine-guanine dinucleotides (CpGs).

69. The rAAV particle or method of claim 63, wherein said expression control element comprises a constitutive or regulatable control element, or a tissue-specific expression control element or promoter.

70. The rAAV particle or method of claim 63, wherein said expression control element comprises an element that confers expression in liver.

71. The rAAV particle or method of claim 63, wherein said expression control element comprises a transthyretin (TTR) promoter (SEQ ID NO:125) or mutant (TTRmut) promoter (SEQ ID NO:126).

72. The rAAV particle or method of claim 63, wherein said ITR comprises one or more ITR of any of: AAV1, AAV2, AAV3, AAV4, AAV5, AAV6, AAV7, AAV8, AAV9, AAV10, AAV11, Rh10, Rh74 or AAV-3B AAV serotypes, or a combination thereof.

73. The rAAV particle or method of any of claims 39-72, wherein said rAAV particle comprises an AAV pseudotype wherein said AAV capsid serotype is different from said ITR serotype.

74. The rAAV particle or method of any of claims 39-72, wherein said rAAV particle comprises an AAV serotype, wherein said AAV capsid serotype is identical to said ITR serotype.

75. A method of treating a mammal having aberrant or undesirable protein expression or activity, comprising administering an effective amount of the rAAV particle, or rAAV particles or pharmaceutical composition of any of claims 37-46 and 49-74 to said mammal, wherein said heterologous nucleic acid sequence is expressed in said mammal, wherein said heterologous nucleic acid sequence encodes an inhibitory RNA selected from a short hairpin (sh)RNA, a microRNA (miRNA), a small or short interfering (si)RNA, a trans-splicing RNA, and an antisense RNA, thereby treating the mammal.

76. A cell comprising a nucleic acid encoding the modified AAV capsid protein of any of claims 1-38.

77. A method of producing the rAAV particle of any of claims 39-48 and 51-74, comprising a. introducing a nucleic acid encoding the modified AAV capsid protein of any of claims 1-38 into a packaging helper cell, said helper cell comprising said vector genome; and b. culturing said helper cell under conditions to produce said rAAV particle.

78. A method of producing the rAAV particle of any of claims 39-48 and 51-74, comprising a. introducing a nucleic acid encoding the modified AAV capsid protein of any of claims 1-38 and introducing said vector genome into a packaging helper cell; and b. culturing said helper cells under conditions to produce said rAAV.

79. The cell or method of any of claims 76-78, wherein said cell comprises mammalian cells.

80. The cell or method of any of claims 76-78, wherein said cell provides helper functions that package said vector into a viral particle.

81. The cell or method of any of claims 76-78, wherein said cell provides AAV helper functions.

82. The cell or method of any of claims 76-78, wherein said cell provides AAV Rep and/or Cap proteins.

83. The cell or method of any of claims 76-78, wherein said cell is stably or transiently transfected with polynucleotide(s) encoding Rep and/or Cap protein sequence(s).

84. The cell or method of any of claims 76-78, wherein said cell provides Rep78 or/and Rep68 proteins.

85. The cell or method of any of claims 76-78, wherein said cell is stably or transiently transfected with Rep78 and Rep68 proteins polynucleotide encoding sequence(s).

86. The cell or method of any of claims 76-78, wherein said cell comprises mammalian cells.

87. The cell or method of any of claims 76-78, wherein said cell comprises HEK-293 cells.
